SEQUENCE LISTING (6 sequences)

SEQ ID NO: 1 - DNA, 90 bp, Artificial sequence
  Biotinylated ssDNA template
  misc_feature (1)..(2): biotin-labeled
  misc_feature (31)..(70): n = dATP, dGTP, dCTP and either PP-dUTP, PBn-dUTP, POP-dUTP, BPE-dUTP or DPP-dUTP
aattttttttttgctctggtcccgctcttgnnnnnnnnnnnnnnnnnnnnnnnnnnnnnnnnnnnnnnnncagtttcaggcaggctaggc

SEQ ID NO: 2 - DNA, 28 bp, Artificial sequence
  Forward PCR primer
atatatatgcctagcctgcctgaaactg

SEQ ID NO: 3 - DNA, 30 bp, Artificial sequence
  Biotinylated reverse PCR primer
  misc_feature (1)..(2): biotin-labeled
aattttttttttgctctggtcccgctcttg

SEQ ID NO: 4 - DNA, 97 bp, Artificial sequence
  Biotinylated ssDNA template
  misc_feature (1)..(2): biotin-labeled
  misc_feature (34)..(73): n = dATP, dGTP, dUTP, and one of the following PP-dCTP, Bn-dCTP, DPP-dCTP, PBn-dCTP, or POP-dCTP
aattttttttctcccttcttctctttcttcttcnnnnnnnnnnnnnnnnnnnnnnnnnnnnnnnnnnnnnnnngtcacggctccgcgacatatatat

SEQ ID NO: 5 - DNA, 24 bp, Artificial sequence
  Forward PCR primer
atatatatgtcgcggagccgtgac

SEQ ID NO: 6 - DNA, 33 bp, Artificial sequence
  Biotinylated reverse PCR primer
  misc_feature (1)..(2): biotin-labeled
aattttttttctcccttcttctctttcttcttc

(19) United States
(12) Patent Application Publication
(10) Pub. No.: US 2025/0283094 A1
(43) Pub. Date: Sep. 11, 2025

(54) Modified Nucleosides

(71) Applicant: Somalogic Operating Co., Inc., Boulder, CO (US)

(72) Inventors: Cate Fowler, Boulder, CO (US); Jeff Carter, Boulder, CO (US); John Rohloff, Boulder, CO (US); Amy Gelinas, Boulder, CO (US); Nebojsa Janjic, Boulder, CO (US)

(73) Assignee: Somalogic Operating Co., Inc., Boulder, CO (US)

(21) Appl. No.: 18/285,736

(22) PCT Filed: Apr. 12, 2022
(86) PCT No.: PCT/US2022/024361

§ 371 Date: Oct. 5, 2023

Related U.S. Application Data
(60) Provisional application No. 63/174,495, filed on Apr. 13, 2021.
(60) Provisional application No. 63/174,792, filed on Apr. 14, 2021.

(51) Int. Cl. C12N 15/115; A61K 31/7088; C07H 19/06; C07H 19/10; C12N 15/10; G01N 33/53

(52) U.S. Cl. CPC C12N 15/115; A61K 31/7088; C07H 19/06; C07H 19/10; C12N 15/1093; G01N 33/5308; C12N 2310/16

(57) ABSTRACT

Compounds comprising 5-position modified pyrimidines are provided. Further, polynucleotides, such as aptamers, comprising at least one 5-position modified pyrimidine are provided. Methods of selecting and using such polynucleotides, such as aptamers, are also provided.

### CROSS-REFERENCE TO RELATED APPLICATIONS

The present application is a national phase entry pursuant to 35 U.S.C. § 371 of International Application No. PCT/US2022/024361, filed Apr. 12, 2022, which claims the benefit of priority of U.S. Provisional Application No. 63/174,495, filed Apr. 13, 2021, and U.S. Provisional Application No. 63/174,792, filed Apr. 14, 2021, each of which is incorporated by reference herein in its entirety for any purpose.

### SEQUENCE LISTING

The present application is filed with a Sequence Listing in electronic format. The Sequence Listing is provided as a file entitled “2024-04-30_01137-0037-00US_ST25.txt” created on Apr. 30, 2024, which is 2,300 bytes in size. The information in the electronic format of the sequence listing is incorporated herein by reference in its entirety.

### FIELD

The present disclosure relates generally to the field of oligonucleotides comprising modified nucleosides, such as aptamers that are capable of binding to target molecules. In some embodiments, the present disclosure relates to oligonucleotides, such as aptamers, that comprise one or more base-modified nucleoside, and methods of making and using such aptamers.

### BACKGROUND

Modified nucleosides have been used as therapeutic agents, diagnostic agents, and for incorporation into oligonucleotides to improve their properties (e.g., stability).

SELEX (Systematic Evolution of Ligands for EXponential Enrichment) is a method for identifying oligonucleotides (referred to as “aptamers”) that selectively bind target molecules. The SELEX process is described, for example, in U.S. Pat. No. 5,270,163. The SELEX method involves the selection and identification of oligonucleotides from a random mixture of oligonucleotides to achieve virtually any desired criterion of binding affinity and selectivity. By introducing specific types of modified nucleosides to the oligonucleotides identified in the course of the SELEX process, the nuclease stability, net charge, hydrophilicity or lipophilicity may be altered to provide differences in the three dimensional structure and target binding capabilities of the oligonucleotides.

There remains a need in the art for alternative modified nucleosides and modified nucleotides, which can be incorporated into oligonucleotides such as aptamers. The present disclosure aims to meet one or more of these needs or provide other benefits.

### SUMMARY

The foregoing and other objects, features, and advantages of the invention will become more apparent from the following detailed description, which proceeds with reference to the accompanying figures.

Certain non-limiting exemplary embodiments are as follows:

Embodiment 1. A compound comprising a 5-position modified pyrimidine nucleoside, or a salt thereof, wherein the 5-position modified pyrimidine is substituted with a moiety comprising two phenyl groups covalently attached to one another by a first linker, and wherein the moiety is covalently linked to the 5-position of the pyrimidine by a second linker.

Embodiment 2. The compound of Embodiment 1, wherein the first linker comprises at least one atom selected from a carbon and oxygen or is a bond.

Embodiment 3. The compound of any one of Embodiments 1-2, wherein the 5-position modified pyrimidine comprises a moiety at the 5-position selected from a phenylbenzyl moiety, a phenoxybenzyl moiety, and a diphenylmethyl moiety.

Embodiment 4. The compound of any one of Embodiments 1-3, wherein the second linker comprises a group selected from an amide linker, a carbonyl linker, a propynyl linker, an alkyne linker, an ester linker, a urea linker, a carbamate linker, a guanidine linker, an amidine linker, a sulfoxide linker, and a sulfone linker.

Embodiment 5. The compound of any one of Embodiments 1-3, wherein the second linker comprises an amide linker.

Embodiment 6. The compound of Embodiment 5, wherein the amide linker further comprises one or more carbon atoms or 1, 2, 3, 4, 5, 6, 7, 8, 9 or 10 carbon atoms.

Embodiment 7. The compound of any one of Embodiments 1-6, comprising a 5-position modified uridine.

Embodiment 8. The compound of any one of Embodiments 1-7, comprising a 5-position modified cytidine.

Embodiment 9. A compound comprising the structure of Formula IA or Formula IB:

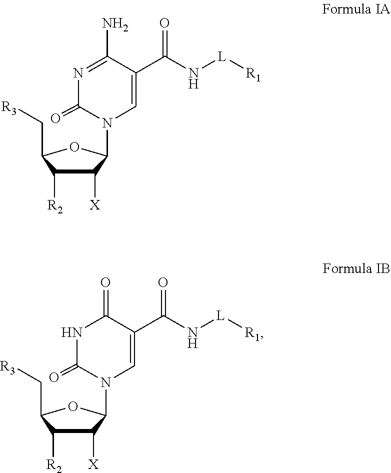

or a salt of either one of these, wherein
each L is independently a —(CH₂)ₙ—, wherein n is 0, 1, 2, 3, 4, 5, 6, 7, 8, 9, or 10;
each R₁ is independently selected from the group consisting of

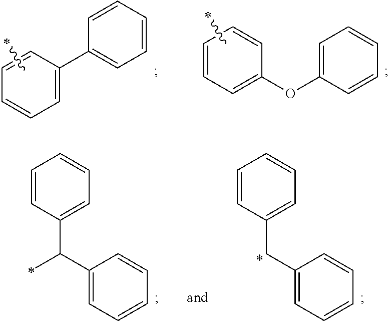

wherein * denotes the point of attachment of the R₁ group to the L group;
each X is independently selected from the group consisting of —H, —OH, —OMe, —O-allyl, —O-ethyl, —O-propyl, —OCH₂CH₂OCH₃, -fluoro, tert-butyldimethylsilyloxy, —NH₂, and -azido;
each R₂ is independently selected from the group consisting of —OH; -acetyl; —OBz; —OP(N(CH₂CH₃)₂)(OCH₂CH₂CN), —OP(N(Rₓ)₂)(OCH₂CH₂CN), wherein each Rₓ is independently (C₁₋₆)alkyl; tert-butyldimethylsilyloxy; —O-ss; —OR; —SR; —ZP(Z′)(Z″)—O—R; wherein ss is a solid support, Z, Z′, and Z″ are each independently selected from O and S, and R is an adjacent nucleotide;
each R₃ is independently selected from the group consisting of —OH, —O-trityl, —O-4,4′-dimethoxytrityl, —O-triphosphate, —OR, —SR, —NH₂, —NHR, and —Z—P(Z′)(Z″)O—R, wherein Z, Z′, and Z″ are each independently selected from O and S, and R is an adjacent nucleotide.

Embodiment 10. The compound of Embodiment 9, wherein n is 1, 2, or 3.

Embodiment 11. The compound of any one of Embodiments 9-10, wherein X is —H.

Embodiment 12. The compound of any one of Embodiments 9-10, wherein X is —OMe.

Embodiment 13. The compound of any one of Embodiments 9-12, wherein each R₁ is independently selected from the group consisting of

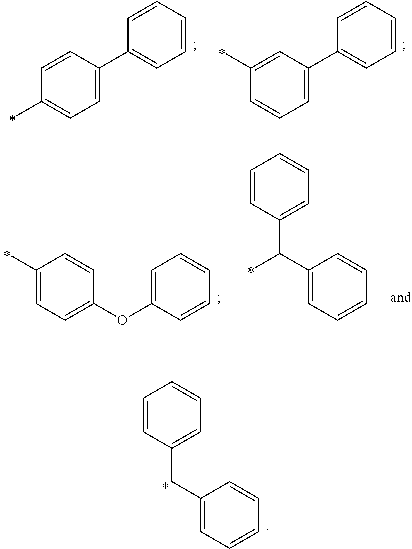

Embodiment 14. The compound of any one of Embodiments 1-13, wherein the 5-position modified pyrimidine is selected from a BPEdU, a 2′-OMe-BPE-U, a 2′-F-BPE-U, a PBndU, a 2′-OMe-PBn-U, a 2′-F-PBn-U, a POPdU, a 2′-OMe-POP-U, a 2′-F-POP-U, a DPPdU, a 2′-OMe-DPP-U, a 2′-F-DPP-U, a DBMdU, a 2′-OMe-DBM-U, a 2′-F-DBM-U, a BHdU, a 2′-OMe-BH-U, a 2′-F-BH-U, a BPEdC, a 2′-OMe-BPE-C, a 2′-F-BPE-C, a PBndC, a 2′-OMe-PBn-C, a 2′-F-PBn-C, a POPdC, a 2′-OMe-POP-C, a 2′-F-POP-C, a DPPdC, a 2′-OMe-DPP-C, a 2′-F-DPP-C, a DBMdC, a 2′-OMe-DBM-C, a 2′-F-DBM-C, a BHdC, a 2′-OMe-BH-C, and a 2′-F-BH-C.

Embodiment 15. A compound comprising the structure of Formula IIA or Formula IIB:

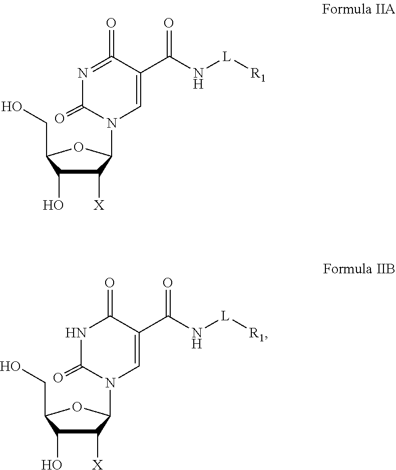

or a salt of either one of these, wherein
each L is independently a —(CH₂)ₙ—, wherein n is 0, 1, 2, 3, 4, 5, 6, 7, 8, 9, or 10;
each R₁ is independently selected from the group consisting of

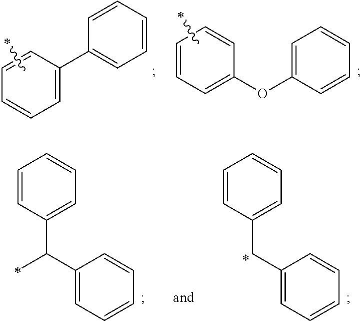

wherein * denotes the point of attachment of the R₁ group to the L group;
each X is independently selected from the group consisting of —H, —OH, —OMe, —O-allyl, —O-ethyl, —O-propyl, —OCH₂CH₂OCH₃, -fluoro, tert-butyldimethylsilyloxy, —NH₂, and -azido.

Embodiment 16. The compound of Embodiment 15, wherein n is 1, 2, or 3.

Embodiment 17. The compound of any one of Embodiments 15-16, wherein X is —H.

Embodiment 18. The compound of any one of Embodiments 15-16, wherein X is —OMe.

Embodiment 19. The compound of any one of Embodiments 15-18, wherein each R₁ is independently selected from the group consisting of

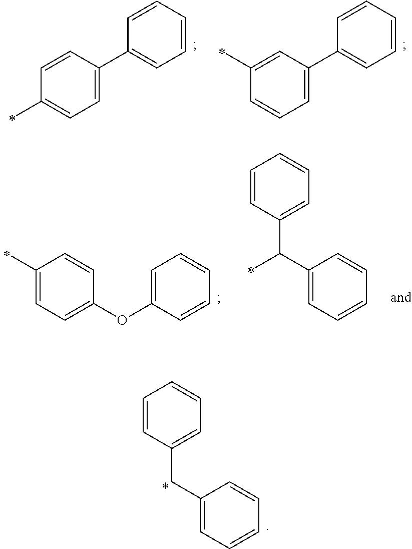

Embodiment 20. A compound comprising the following structure:

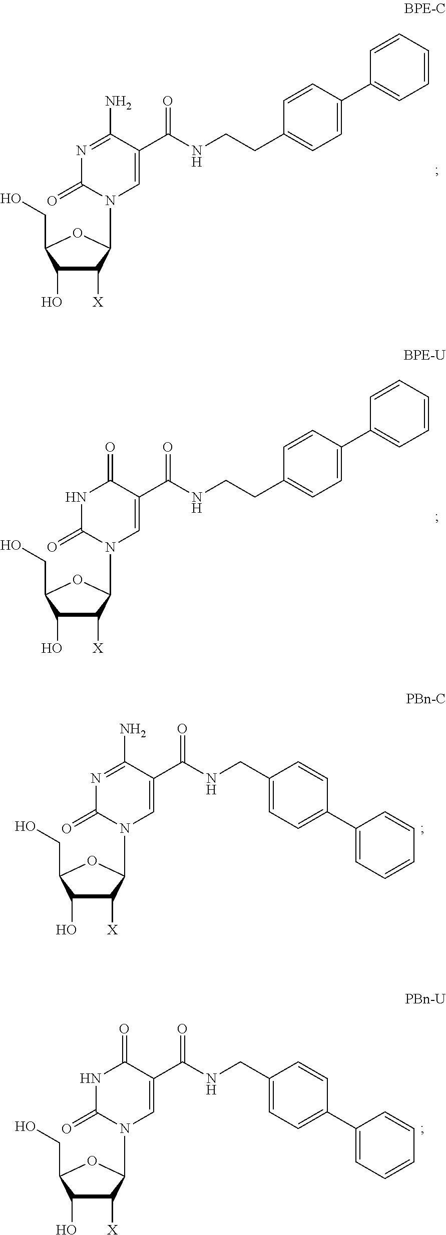

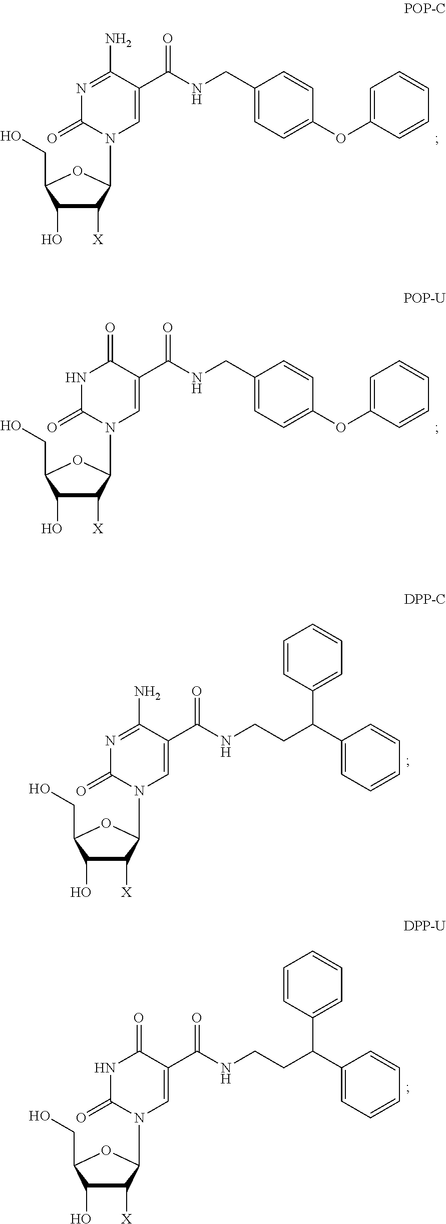

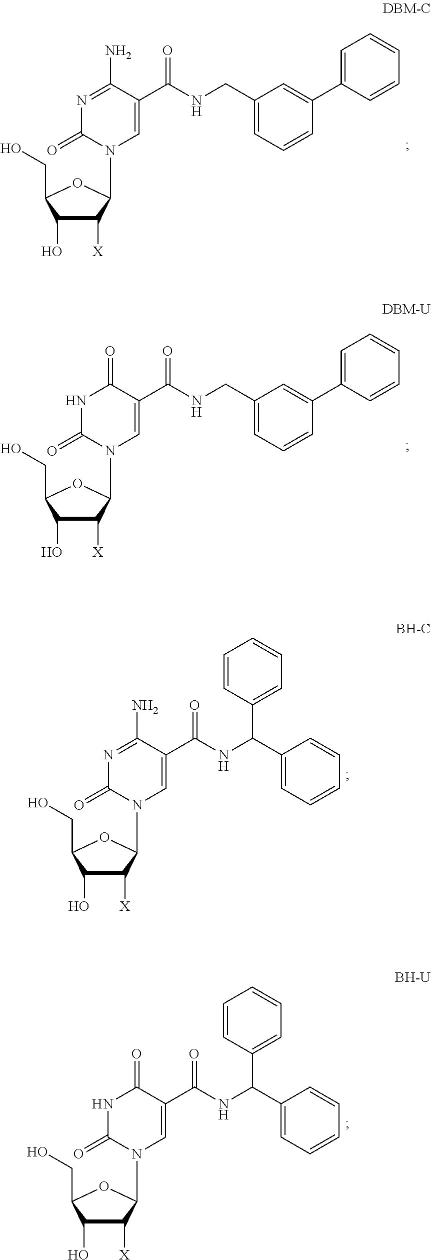

or a salt of any one of these;
wherein each X is independently selected from the group consisting of —H, —OH, —O-methyl, —O-allyl, —O-ethyl, —O-propyl, —OCH₂CH₂OCH₃, -fluoro, tert-butyldimethylsilyloxy, —NH₂, and -azido.

Embodiment 21. The compound of Embodiment 20, wherein X is —H.

Embodiment 22. The compound of Embodiment 20, wherein X is —OMe.

Embodiment 23. An oligonucleotide comprising the compound of any one of Embodiments 1-14.

Embodiment 24. The oligonucleotide of Embodiment 23, which comprises RNA, DNA, or a combination thereof.

Embodiment 25. The oligonucleotide of any one of Embodiments 23-24, which is 15 to 100, or 15 to 90, or 15 to 80, or 15 to 70, or 15 to 60, or 15 to 50, or 20 to 100, or 20 to 90, or 20 to 80, or 20 to 70, or 20 to 60, or 20 to 50, or 30 to 100, or 30 to 90, or 30 to 80, or 30 to 70, or 30 to 60, or 30 to 50, or 40 to 100, or 40 to 90, or 40 to 80, or 40 to 70, or 40 to 60, or 40 to 50 nucleotides in length.

Embodiment 26. The oligonucleotide of any one of Embodiments 23-25, which is an aptamer that binds a target.

Embodiment 27. An aptamer comprising the compound of any one of Embodiments 1-14.

Embodiment 28. The aptamer of any one of Embodiments 26-27, wherein the aptamer is 15 to 100, or 15 to 90, or 15 to 80, or 15 to 70, or 15 to 60, or 15 to 50, or 20 to 100, or 20 to 90, or 20 to 80, or 20 to 70, or 20 to 60, or 20 to 50, or 30 to 100, or 30 to 90, or 30 to 80, or 30 to 70, or 30 to 60, or 30 to 50, or 40 to 100, or 40 to 90, or 40 to 80, or 40 to 70, or 40 to 60, or 40 to 50 nucleotides in length.

Embodiment 29. The aptamer of any one of Embodiments 26-28, comprising a 5-position modified pyrimidine selected from a BPEdU, a 2′-OMe-BPE-U, a 2′-F-BPE-U, a PBndU, a 2′-OMe-PBn-U, a 2′-F-PBn-U, a POPdU, a 2′-OMe-POP-U, a 2′-F-POP-U, a DPPdU, a 2′-OMe-DPP-U, a 2′-F-DPP-U, a DBMdU, a 2′-OMe-DBM-U, a 2′-F-DBM-U, a BHdU, a 2′-OMe-BH-U, a 2′-F-BH-U, a BPEdC, a 2′-OMe-BPE-C, a 2′-F-BPE-C, a PBndC, a 2′-OMe-PBn-C, a 2′-F-PBn-C, a POPdC, a 2′-OMe-POP-C, a 2′-F-POP-C, a DPPdC, a 2′-OMe-DPP-C, a 2′-F-DPP-C, a DBMdC, a 2′-OMe-DBM-C, a 2′-F-DBM-C, a BHdC, a 2′-OMe-BH-C, and a 2′-F-BH-C.

Embodiment 30. The aptamer of any one of Embodiments 26-29, comprising at least one 5-position modified uridine selected from a BPEdU, a 2′-OMe-BPE-U, a 2′-F-BPE-U, a PBndU, a 2′-OMe-PBn-U, a 2′-F-PBn-U, a POPdU, a 2′-OMe-POP-U, a 2′-F-POP-U, a DPPdU, a 2′-OMe-DPP-U, a 2′-F-DPP-U, a DBMdU, a 2′-OMe-DBM-U, a 2′-F-DBM-U, a BHdU, a 2′-OMe-BH-U, a 2′-F-BH-U, and at least one 5-position modified cytidine selected from a BPEdC, a 2′-OMe-BPE-C, a 2′-F-BPE-C, a PBndC, a 2′-OMe-PBn-C, a 2′-F-PBn-C, a POPdC, a 2′-OMe-POP-C, a 2′-F-POP-C, a DPPdC, a 2′-OMe-DPP-C, a 2′-F-DPP-C, a DBMdC, a 2′-OMe-DBM-C, a 2′-F-DBM-C, a BHdC, a 2′-OMe-BH-C, and a 2′-F-BH-C.

Embodiment 31. The aptamer of any one of Embodiments 26-30, wherein the aptamer comprises a region at the 5′ end of the aptamer that is at least 10, at least 15, at least 20, at least 25 or at least 30 nucleotides in length, or 5 to 30, 10 to 30, 15 to 30, 5 to 20, or 10 to 20 nucleotides in length, wherein the region at the 5′ end of the aptamer lacks 5-position modified pyrimidines.

Embodiment 32. The aptamer of any one of Embodiments 26-31, wherein the aptamer comprises a region at the 3′ end of the aptamer that is at least 10, at least 15, at least 20, at least 25 or at least 30 nucleotides in length, or 5 to 30, 10 to 30, 15 to 30, 5 to 20, or 10 to 20 nucleotides in length, wherein the region at the 3′ end of the aptamer lacks 5-position modified pyrimidines.

Embodiment 33. An aptamer comprising at least one first 5-position modified pyrimidine and at least one second 5-position modified pyrimidine, wherein the first 5-position modified pyrimidine and the second 5-position modified pyrimidine are different 5-position modified pyrimidines, and wherein the at least one first 5-position modified pyrimidine is a compound according to any one of Embodiments 1-14.

Embodiment 34. The aptamer of Embodiment 33, wherein the at least one second 5-position modified pyrimidine is selected from the group consisting of one or more of BndC, 2′-OMe-Bn-C, 2′-F-Bn-C, PEdC, 2′-OMe-PE-C, 2′-F-PE-C, PPdC, 2′-OMe-PP-C, 2′-F-PP-C, NapdC, 2′-OMe-Nap-C, 2′-F-Nap-C, 2NapdC, 2′-OMe-2Nap-C, 2′-F-2Nap-C, NEdC, 2′-OMe-NE-C, 2′-F-NE-C, 2NEdC, 2′-OMe-2NE-C, 2′-F-2NE-C, TyrdC, 2′-OMe-Tyr-C, 2′-F-Tyr-C, BndU, 2′-OMe-Bn-U, 2′-F-Bn-U, NapdU, 2′-OMe-Nap-U, 2′-F-Nap-U, PEdU, 2′-OMe-PE-U, 2′-F-PE-U, IbdU, 2′-OMe-Ib-U, 2′-F-Ib-U, FBndU, 2′-OMe-FBn-U, 2′-F-FBn-U, 2NapdU, 2′-OMe-2Nap-U, 2′-F-2Nap-U, NEdU, 2′-OMe-NE-U, 2′-F-NE-U, MBndU, 2′-OMe-MBn-U, 2′-F-MBn-U, BFdU, 2′-OMe-BF-U, 2′-F-BF-U, BTdU, 2′-OMe-BT-U, 2′-F-BT-U, PPdU, 2′-OMe-PP-U, 2′-F-PP-U, MOEdU, 2′-OMe-MOE-U, 2′-F-MOE-U, TyrdU, 2′-OMe-Tyr-U, 2′-F-Tyr-U, TrpdU, 2′-OMe-Trp-U, 2′-F-Trp-U, ThrdU, 2′-OMe-Thr-U, and 2′-F-Thr-U.

Embodiment 35. The aptamer of any one of Embodiments 33-34, wherein the at least one second 5-position modified pyrimidine is selected from the group consisting of one or more of NapdC, 2′-OMe-Nap-C, 2′-F-Nap-C, 2NapdC, 2′-OMe-2Nap-C, 2′-F-2Nap-C, TyrdC, 2′-OMe-Tyr-C, 2′-F-Tyr-C, PPdC, 2′-OMe-PP-C, 2′-F-PP-C, NapdU, 2′-OMe-Nap-U, 2′-F-Nap-U, PPdU, 2′-OMe-PP-U, 2′-F-PP-U, MOEdU, 2′-OMe-MOE-U, 2′-F-MOE-U, TyrdU, 2′-OMe-Tyr-U, 2′-F-Tyr-U, TrpdU, 2′-OMe-Trp-U, 2′-F-Trp-U, ThrdU, 2′-OMe-Thr-U, and 2′-F-Thr-U.

Embodiment 36. The aptamer of any one of Embodiments 26-35, wherein the aptamer has improved nuclease stability and/or a longer half-life in human serum and/or improved affinity and/or improved off-rate compared to an aptamer of the same length and nucleobase sequence that comprises an unmodified pyrimidine in place of the 5-position modified pyrimidine.

Embodiment 37. A composition comprising a plurality of aptamers of any one of Embodiments 26-36.

Embodiment 38. The composition of Embodiment 37, wherein each aptamer comprises a random region.

Embodiment 39. The composition of Embodiment 38, wherein the random region is 20 to 100, or 20 to 90, or 20 to 80, or 20 to 70, or 20 to 60, or 20 to 50, or 20 to 40, or 30 to 100, or 30 to 90, or 30 to 70, or 30 to 60, or 30 to 50, or 30 to 40 nucleotides in length.

Embodiment 40. A composition comprising an aptamer and a target, wherein the aptamer and the target are capable of forming a complex, and wherein the aptamer is an aptamer of any one of Embodiments 26-35.

Embodiment 41. A composition comprising a first aptamer, a second aptamer, and a target, wherein the first aptamer, the second aptamer, and the target are capable of forming a trimer complex; and wherein the first aptamer is an aptamer comprising a compound of any one of Embodiments 1-14; and wherein the second aptamer comprises at least one second 5-position modified pyrimidine.

Embodiment 42. The composition of Embodiment 41, wherein the target is selected from a protein, a peptide, a carbohydrate, a small molecule, a cell and a tissue.

Embodiment 43. The composition of any one of Embodiments 41-42, wherein the target is a target protein selected from IL-33, XIAP, K-Ras, and TNF-alpha.

Embodiment 44. A pharmaceutical composition comprising at least one aptamer of any one of Embodiments 26-35, or a pharmaceutically acceptable salt thereof, and a pharmaceutically acceptable carrier.

Embodiment 45. The pharmaceutical composition of Embodiment 44, for treating or preventing a disease or condition mediated by a protein selected from IL-33, XIAP, K-Ras, and TNF-alpha.

Embodiment 46. A method of treating or preventing a disease or condition in a subject, comprising administering to a subject in need thereof an aptamer of any one of Embodiments 26-35 or a pharmaceutical composition of any one of Embodiments 44-45.

Embodiment 47. The method of Embodiment 46, wherein the disease or condition is mediated by a protein selected from IL-33, XIAP, K-Ras, and TNF-alpha.

Embodiment 48. The method of any one of Embodiments 46-47, wherein the disease or condition is traumatic brain injury (TBI) or rheumatoid arthritis.

Embodiment 49. A method comprising: (a) contacting an aptamer capable of binding to a target molecule with a sample; (b) incubating the aptamer with the sample to allow an aptamer-target complex to form; (c) enriching for the aptamer-target complex in the sample; and (d) detecting for the presence of the aptamer, the aptamer-target complex, or the target molecule, wherein the detection of the aptamer, the aptamer-target complex, or the target molecule indicates that the target molecule is present in the sample, and wherein the lack of detection of the aptamer, the aptamer-target complex, or the target molecule indicates that the target molecule is not present in the sample; wherein the aptamer comprises a compound of any one of Embodiments 1-14 or is an aptamer of any one of Embodiments 26-35.

Embodiment 50. The method of Embodiment 49, wherein the method comprises at least one additional step selected from: adding a competitor molecule to the sample; capturing the aptamer-target complex on a solid support; and adding a competitor molecule and diluting the sample; wherein the at least one additional step occurs after step (a) or step (b).

Embodiment 51. The method of Embodiment 50, wherein the competitor molecule is selected from a polyanionic competitor.

Embodiment 52. The method of Embodiment 51, wherein the polyanionic competitor is selected from an oligonucleotide, polydextran, DNA, heparin, and dNTPs.

Embodiment 53. The method of Embodiment 52, wherein polydextran is dextran sulfate; and DNA is herring sperm DNA or salmon sperm DNA.

Embodiment 54. The method of any one of Embodiments 49-53, wherein the target molecule is selected from a protein, a peptide, a carbohydrate, a small molecule, a cell and a tissue.

Embodiment 55. The method of any one of Embodiments 49-54, wherein the sample is selected from whole blood, leukocytes, peripheral blood mononuclear cells, plasma, serum, sputum, breath, urine, semen, saliva, meningial fluid, amniotic fluid, glandular fluid, lymph fluid, nipple aspirate, bronchial aspirate, synovial fluid, joint aspirate, cells, a cellular extract, stool, tissue, a tissue biopsy, and cerebrospinal fluid.

Embodiment 56. A method for detecting a target in a sample comprising: (a) contacting the sample with a first aptamer to form a mixture, wherein the first aptamer is capable of binding to the target to form a first complex; (b) incubating the mixture under conditions that allow for the first complex to form; (c) contacting the mixture with a second aptamer, wherein the second aptamer is capable of binding the first complex to form a second complex; (d) incubating the mixture under conditions that allow for the second complex to form; (e) detecting for the presence or absence of the first aptamer, the second aptamer, the target, the first complex or the second complex in the mixture, wherein the presence of the first aptamer, the second aptamer, the target, the first complex or the second complex indicates that the target is present in the sample; wherein the first aptamer comprises a compound of any one of Embodiments 1-14; and wherein the second aptamer comprises at least one second 5-position modified pyrimidine; wherein the first aptamer, the second aptamer and the target are capable of forming a trimer complex.

Embodiment 57. The method of Embodiment 56, wherein the target molecule is selected from a protein, a peptide, a carbohydrate, a small molecule, a cell and a tissue.

Embodiment 58. The method of any one of Embodiments 56-57, wherein the first aptamer, the second aptamer and the target are capable of forming a trimer complex.

Embodiment 59. The method of any one of Embodiments 56-58, wherein the second aptamer comprises at least one second 5-position modified pyrimidine selected from the group consisting of one or more of BndC, 2′-OMe-Bn-C, PEdC, 2′-OMe-PE-C, PPdC, 2′-OMe-PP-C, NapdC, 2′-OMe-Nap-C, 2NapdC, 2′-OMe-2Nap-C, NEdC, 2′-OMe-NE-C, 2NEdC, 2′-OMe-2NE-C, TyrdC, 2′-OMe-Tyr-C, BndU, 2′-OMe-Bn-U, NapdU, 2′-OMe-Nap-U, PEdU, 2′-OMe-PE-U, IbdU, 2′-OMe-Ib-U, FBndU, 2′-OMe-FBn-U, 2NapdU, 2′-OMe-2Nap-U, NEdU, 2′-OMe-NE-U, MBndU, 2′-OMe-MBn-U, BFdU, 2′-OMe-BF-U, BTdU, 2′-OMe-BT-U, PPdU, 2′-OMe-PP-U, MOEdU, 2′-OMe-MOE-U, TyrdU, 2′-OMe-Tyr-U, TrpdU, 2′-OMe-Trp-U, ThrdU, and 2′-OMe-Thr-U.

Embodiment 60. The method of any one of Embodiments 56-59, wherein the second aptamer comprises at least one second 5-position modified pyrimidine selected from the group consisting of one or more of NapdC, 2′-OMe-Nap-C, 2NapdC, 2′-OMe-2Nap-C, TyrdC, 2′-OMe-Tyr-C, PPdC, 2′-OMe-PP-C, NapdU, 2′-OMe-Nap-U, PPdU, 2′-OMe-PP-U, MOEdU, 2′-OMe-MOE-U, TyrdU, 2′-OMe-Tyr-U, TrpdU, 2′-OMe-Trp-U, ThrdU, and 2′-OMe-Thr-U.

Embodiment 61. A method for identifying one or more aptamers capable of binding to a target molecule comprising: (a) contacting a library of aptamers with the target molecule to form a mixture, and allowing for the formation of an aptamer-target complex, wherein the aptamer-target complex forms when an aptamer has affinity for the target molecule; (b) partitioning the aptamer-target complex from the remainder of the mixture (or enriching for the aptamer-target complex); (c) dissociating the aptamer-target complex; and (d) identifying the one or more aptamers capable of binding to the target molecule; wherein the library of aptamers comprises a plurality of polynucleotides, and which is the composition of any one of Embodiments 37-43.

Embodiment 62. The method of Embodiment 61, wherein each polynucleotide comprises a fixed region at the 5′ end of the polynucleotide.

Embodiment 63. The method of Embodiment 62, wherein the fixed region at the 5′ end of each polynucleotide is at least 10, at least 15, at least 20, at least 25 or at least 30 nucleotides in length, or 5 to 30, 10 to 30, 15 to 30, 5 to 20, or 10 to 20 nucleotides in length.

Embodiment 64. The method of any one of Embodiments 61-63, wherein each polynucleotide comprises a fixed region at the 3′ end of the polynucleotide.

Embodiment 65. The method of Embodiment 64, wherein the fixed region at the 3′ end of the polynucleotide is at least 10, at least 15, at least 20, at least 25 or at least 30 nucleotides in length, or 5 to 30, 10 to 30, 15 to 30, 5 to 20, or 10 to 20 nucleotides in length.

Embodiment 66. The method of any one of Embodiments 61-65, wherein each polynucleotide comprises a random region.

Embodiment 67. The method of Embodiment 66, wherein the random region is 20 to 100, or 20 to 90, or 20 to 80, or 20 to 70, or 20 to 60, or 20 to 50, or 20 to 40, or 30 to 100, or 30 to 90, or 30 to 70, or 30 to 60, or 30 to 50, or 30 to 40 nucleotides in length.

Embodiment 68. The method of any one of Embodiments 61-67, wherein each polynucleotide is 15 to 100, or 15 to 90, or 15 to 80, or 15 to 70, or 15 to 60, or 15 to 50, or 20 to 100, or 20 to 90, or 20 to 80, or 20 to 70, or 20 to 60, or 20 to 50, or 30 to 100, or 30 to 90, or 30 to 80, or 30 to 70, or 30 to 60, or 30 to 50, or 40 to 100, or 40 to 90, or 40 to 80, or 40 to 70, or 40 to 60, or 40 to 50 nucleotides in length.

Embodiment 69. The method of any one of Embodiments 61-68, wherein each polynucleotide is an aptamer that binds a target, and wherein the library comprises at least 1000 aptamers, wherein each aptamer comprises a different nucleotide sequence.

Embodiment 70. The method of any one of Embodiments 61-69, wherein steps (a), (b), and/or (c) are repeated at least one time, two times, three times, four times, five times, six times, seven times, eight times, nine times, or ten times.

Embodiment 71. The method of any one of Embodiments 61-70, wherein the one or more aptamers capable of binding to the target molecule is amplified.

Embodiment 72. The method of any one of Embodiments 61-71, wherein the mixture comprises a polyanionic competitor molecule.

Embodiment 73. The method of Embodiment 72, wherein the polyanionic competitor is selected from an oligonucleotide, polydextran, DNA, heparin and dNTPs.

Embodiment 74. The method of Embodiment 73, wherein polydextran is dextran sulfate; and DNA is herring sperm DNA or salmon sperm DNA.

Embodiment 75. The method of any one of Embodiments 61-74, wherein the target molecule is selected from a protein, a peptide, a carbohydrate, a small molecule, a cell and a tissue.

Embodiment 76. The compound of any one of Embodiments 1-14, the aptamer of any one of Embodiments 26-35, the composition of any one of Embodiments 37-45, or the method of any one of Embodiments 46-75, wherein the 5-position modified pyrimidine is capable of being incorporated by a polymerase enzyme.

Embodiment 77. A kit comprising the compound of any one of Embodiments 1-14, the compound of any one of Embodiments 15-22, the oligonucleotide of any one of Embodiments 23-25, the aptamer of any one of Embodiments 26-35, the composition of any one of Embodiments 37-43, and optionally one or more of (a) a pharmaceutically acceptable carrier, such as a solvent or solution; (b) a pharmaceutically acceptable excipient, such as a stabilizer or buffer; (c) at least one container, vial, or apparatus for holding and/or mixing the kit components; and (d) a delivery apparatus.

Embodiment 78. The kit of Embodiment 77, optionally further comprising one or more of (e) labeling agents useful to detect a target molecule that is bound to an aptamer; (f) a solid support, such as a microarray or bead; and (g) reagents related to quantitation of polymerase chain reaction products, such as intercalating fluorescent dyes or fluorescent DNA probes.

Embodiment 79. A compound comprising the structure of Formula III, Formula IV, or Formula V:

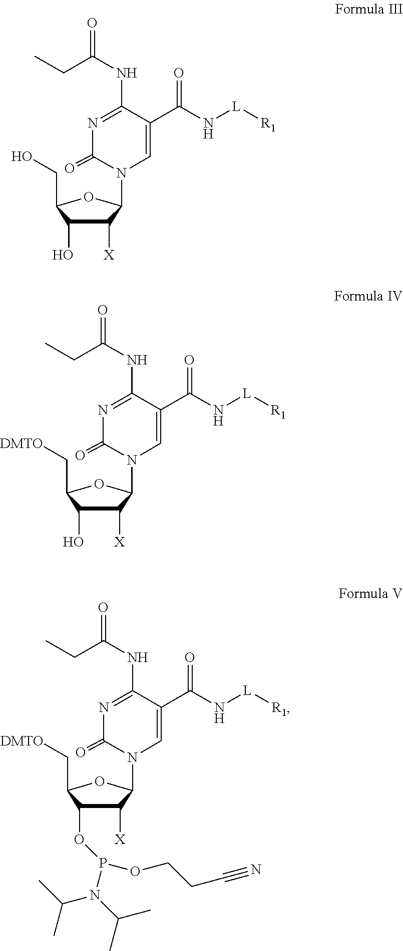

or a salt of any one of these, wherein:
each L is independently a —(CH₂)ₙ—, wherein n is 0, 1, 2, 3, 4, 5, 6, 7, 8, 9, or 10;
each R₁ is independently selected from

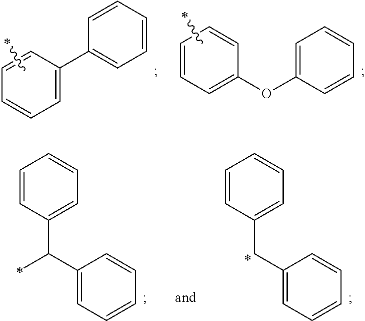

wherein * denotes the point of attachment of the R₁ group to the L group; and
each X is independently selected from —H, —OH, —OMe,
   —O-allyl, —O-ethyl, —O-propyl, —OCH₂CH₂OCH₃, -fluoro, tert-butyldimethylsilyloxy, —NH₂, and -azido.

Embodiment 80. The compound of claim 79, wherein n is 1, 2, or 3.

Embodiment 81. The compound of any one of claims 79-80, wherein X is —H.

Embodiment 82. The compound of any one of claims 79-80, wherein X is —OMe.

Embodiment 83. The compound of any one of claims 79-82, wherein R₁ is selected from

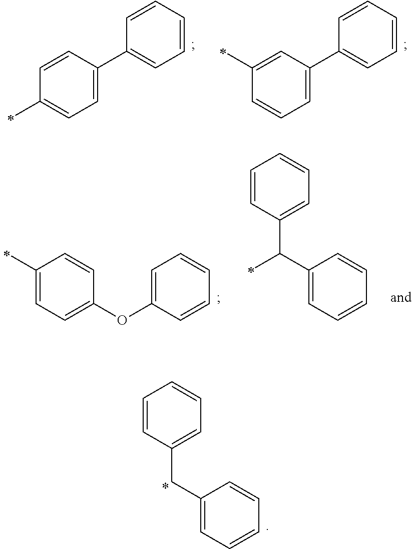

### BRIEF DESCRIPTION OF THE DRAWINGS

FIG. 1A-1I. Dose-dependent binding of round 7 enriched SELEX pools to various protein targets. For each binding curve, the fraction of library-protein complex was plotted as a function of protein concentration. Equilibrium binding constants (Kd values) were determined by fitting the data to a four-parameter sigmoid dose-response model. (A) Comparison of round 7 enriched SELEX pools for PP-dUTP and DPP-dUTP containing libraries for the XIAP target. (B) Comparison of round 7 enriched SELEX pools for PP-dUTP and PBn-dUTP containing libraries for the XIAP target. (C) Comparison of round 7 enriched SELEX pools for PP-dUTP and DPP-dUTP containing libraries for the IL-33 target. (D) Comparison of round 7 enriched SELEX pools for PP-dUTP and PBn-dUTP containing libraries for the IL-33 target. (E) Comparison of round 7 enriched SELEX pools for PP-dUTP and POP-dUTP containing libraries for the K-Ras target. (F) Comparison of round 7 enriched SELEX pools for PP-dUTP and DPP-dUTP containing libraries for the K-Ras target. (G) Comparison of round 7 enriched SELEX pools for PP-dUTP and PBn-dUTP containing libraries for the K-Ras target. (H) Comparison of round 7 enriched SELEX pools for PP-dUTP and BPE-dUTP containing libraries for the K-Ras target. (I) Comparison of round 7 enriched SELEX pools for PP-dUTP and POP-dUTP containing libraries for the TNF-alpha target.

FIG. 2. Certain exemplary 5-position modified uridines and cytidines that may be incorporated into aptamers.

FIG. 3. Certain exemplary modifications that may be present at the 5-position of uridine. The chemical structure of the C-5 modification includes the exemplary amide linkage that links the modification to the 5-position of uridine. The 5-position moieties shown include two phenyl groups covalently attached to one another. The 5-position moieties shown include a phenylbenzyl moiety (e.g., BPE, PBnd, DBM), a 4-phenoxybenzyl moiety (e.g., POP), a diphenylpropyl moiety (e.g., DPP), a benzhydryl moiety (e.g., BH).

FIG. 4. Certain exemplary modifications that may be present at the 5-position of cytidine. The chemical structure of the C-5 modification includes the exemplary amide linkage that links the modification to the 5-position of cytidine. The 5-position moieties shown include two phenyl groups covalently attached to one another. The 5-position moieties shown include a phenylbenzyl moiety (e.g., BPE, PBnd, DBM), a 4-phenoxybenzyl moiety (e.g., POP), a diphenylpropyl moiety (e.g., DPP), a benzhydryl moiety (e.g., BH).

FIG. 5. Certain exemplary modifications that may be present at the 5-position of uridine. The chemical structure of the C-5 modification includes the exemplary amide linkage that links the modification to the 5-position of the uridine. The 5-position moieties shown include a benzyl moiety (e.g., Bn, PE and a PP), a naphthyl moiety (e.g., Nap, 2Nap, NE), a butyl moiety (e.g, iBu), a fluorobenzyl moiety (e.g., FBn), a tyrosyl moiety (e.g., a Tyr), a 3,4-methylenedioxy benzyl (e.g., MBn), a morpholino moiety (e.g., MOE), a benzofuranyl moiety (e.g., BF), an indole moiety (e.g, Trp) and a hydroxypropyl moiety (e.g., Thr).

FIG. 6. Certain exemplary modifications that may be present at the 5-position of cytidine. The chemical structure of the C-5 modification includes the exemplary amide linkage that links the modification to the 5-position of the cytidine. The 5-position moieties shown include a benzyl moiety (e.g., Bn, PE and a PP), a naphthyl moiety (e.g., Nap, 2Nap, NE, and 2NE) and a tyrosyl moiety (e.g., a Tyr).

FIG. 7A-7F. Dose-dependent binding of round 8 (TNFα and sL-selectin) or round 6 (B7-H4) enriched SELEX pools to various protein targets. For each binding curve, the fraction of library-protein complex was plotted as a function of protein concentration. Equilibrium binding constants (Kd values) were determined by fitting the data to a four-parameter sigmoid dose-response model. (A) Comparison of round 8 enriched SELEX pools for Bn-dCTP, PP-dCTP, and DPP-dCTP containing libraries for the TNFα target. (B) Comparison of round 8 enriched SELEX pools for Bn-dCTP, PP-dCTP, and PBn-dCTP containing libraries for the TNFα target. (C) Comparison of round 8 enriched SELEX pools for Bn-dCTP, PP-dCTP, and POP-dCTP containing libraries for the TNFα target. (D) Comparison of round 8 enriched SELEX pools for Bn-dCTP and PBn-dCTP containing libraries for the B7-H4 target. (E) Comparison of round 8 enriched SELEX pools for PP-dCTP and PBn-dCTP containing libraries for the sL-selectin target. (F) Comparison of round 8 enriched SELEX pools for PP-dCTP and POP-dCTP containing libraries for the sL-selectin target.

### DETAILED DESCRIPTION

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean±20% of the indicated range, value, or structure, unless otherwise indicated. As used herein, the terms “include” and “comprise” are open ended and are used synonymously.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

As used herein, the term “nucleotide” refers to a ribonucleotide or a deoxyribonucleotide, or a modified form thereof, as well as an analog thereof. Nucleotides include species that include purines (e.g., adenine, hypoxanthine, guanine, and their derivatives and analogs) as well as pyrimidines (e.g., cytosine, uracil, thymine, and their derivatives and analogs). As used herein, the term “cytidine” is used generically to refer to a ribonucleotide, deoxyribonucleotide, or modified ribonucleotide comprising a cytosine base, unless specifically indicated otherwise. The term “cytidine” includes 2′-modified cytidines, such as 2′-fluoro, 2′-methoxy, etc. Similarly, the term “modified cytidine” or a specific modified cytidine also refers to a ribonucleotide, deoxyribonucleotide, or modified ribonucleotide (such as 2′-fluoro, 2′-methoxy, etc.) comprising the modified cytosine base, unless specifically indicated otherwise.

The term “uridine” is used generically to refer to a ribonucleotide, deoxyribonucleotide, or modified ribonucleotide comprising a uracil base, unless specifically indicated otherwise. The term “uridine” includes 2′-modified uridines, such as 2′-fluoro, 2′-methoxy, etc. Similarly, the term “modified uridine” or a specific modified uridine also refers to a ribonucleotide, deoxyribonucleotide, or modified ribonucleotide (such as 2′-fluoro, 2′-methoxy, etc.) comprising the modified uracil base, unless specifically indicated otherwise.

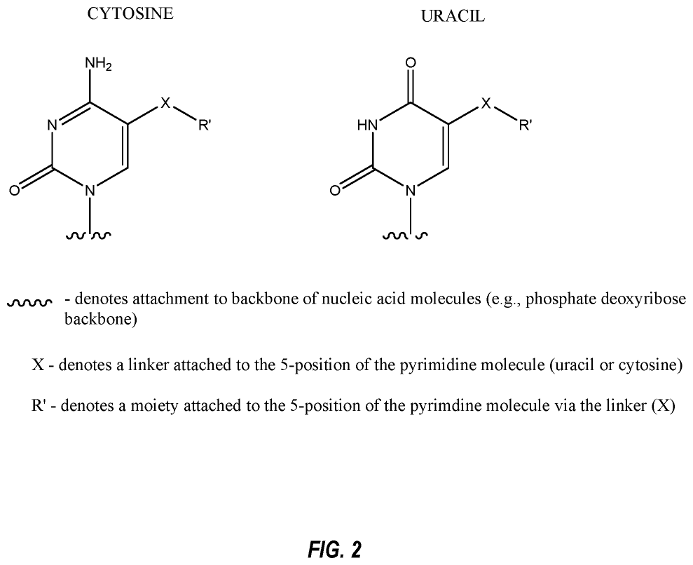

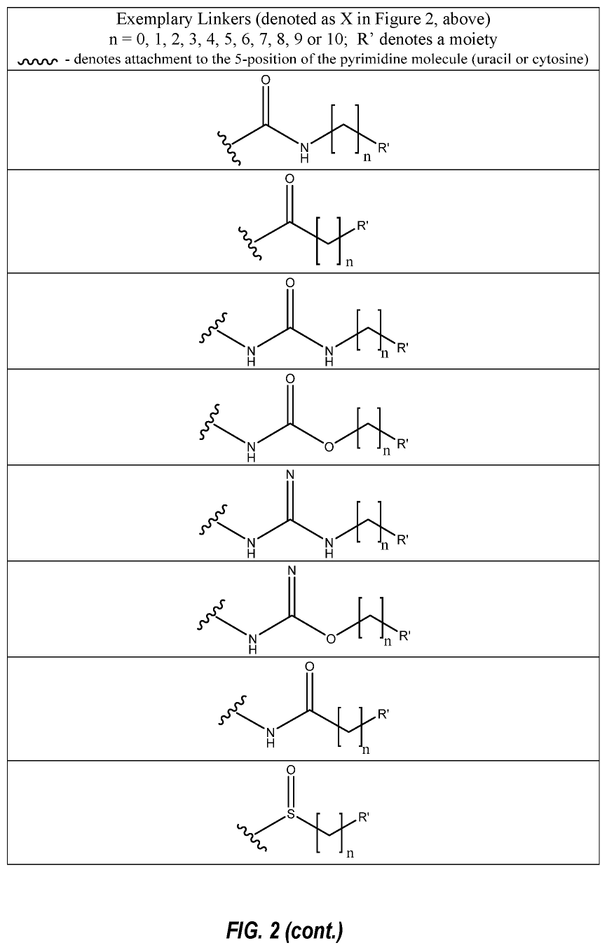

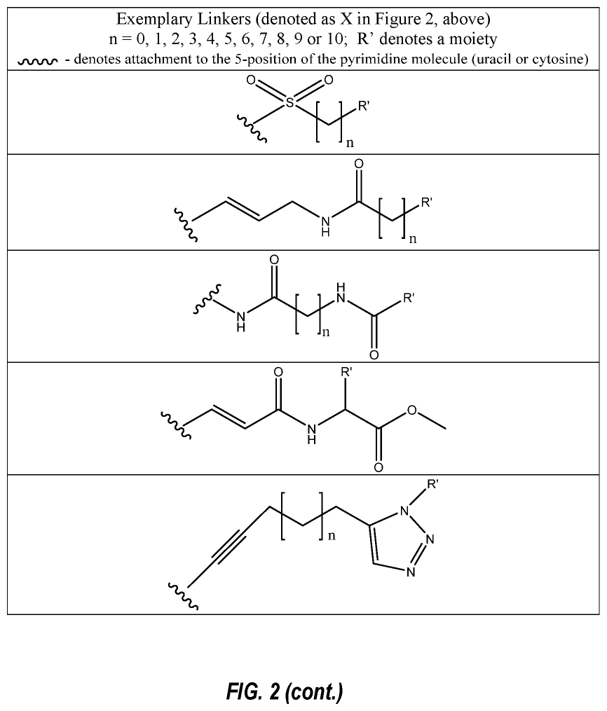

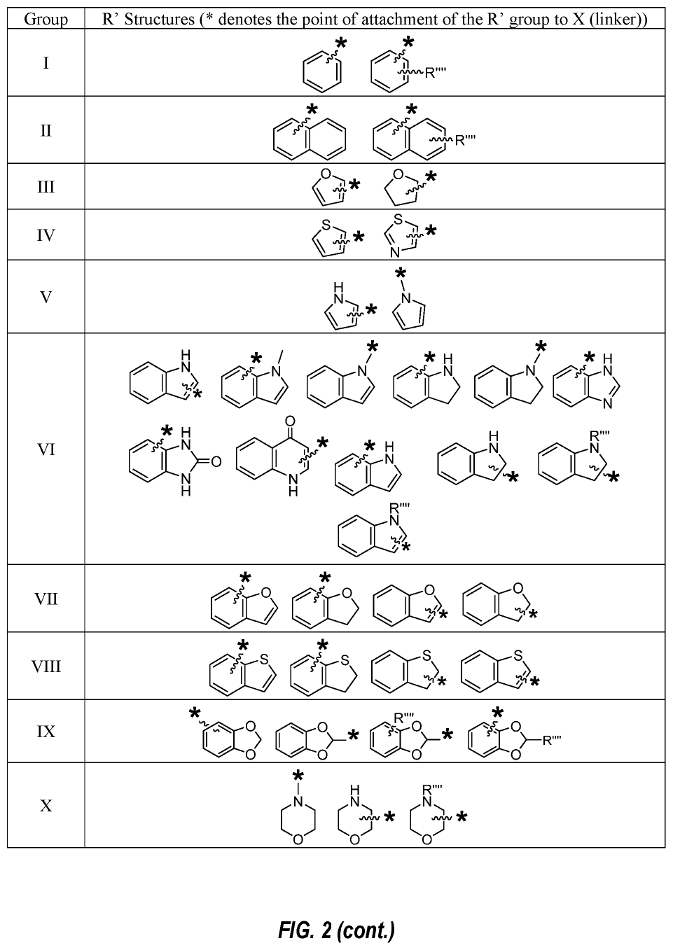

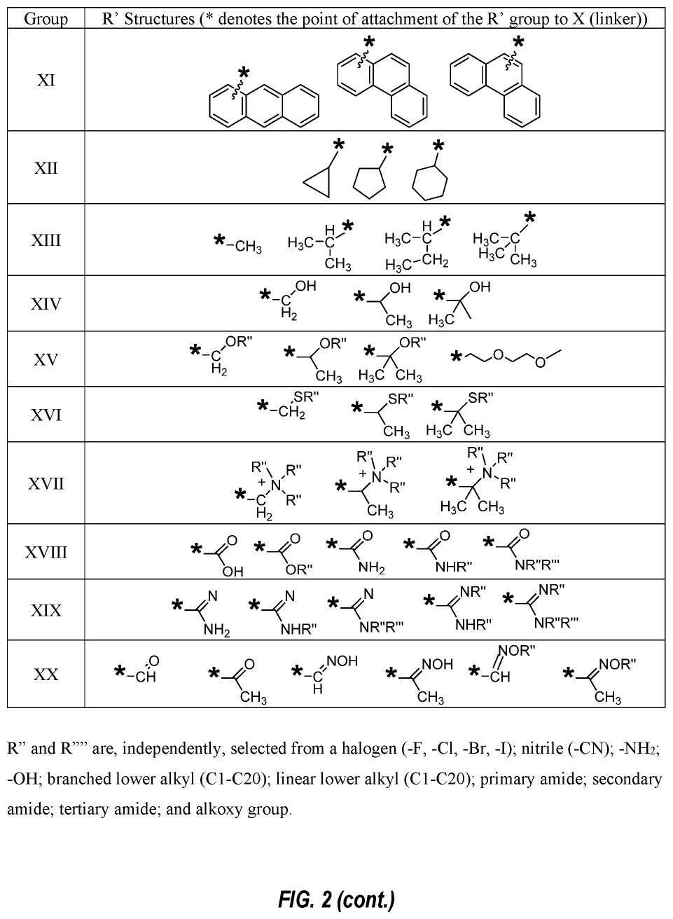

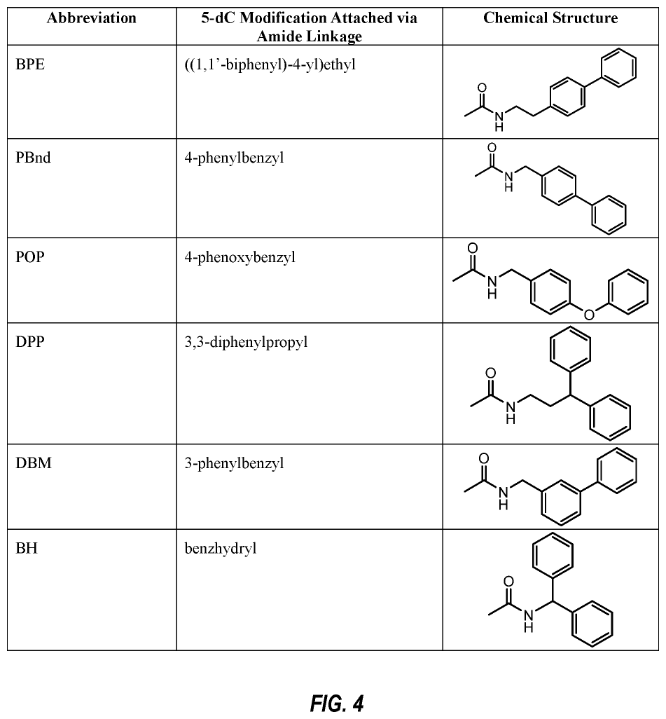

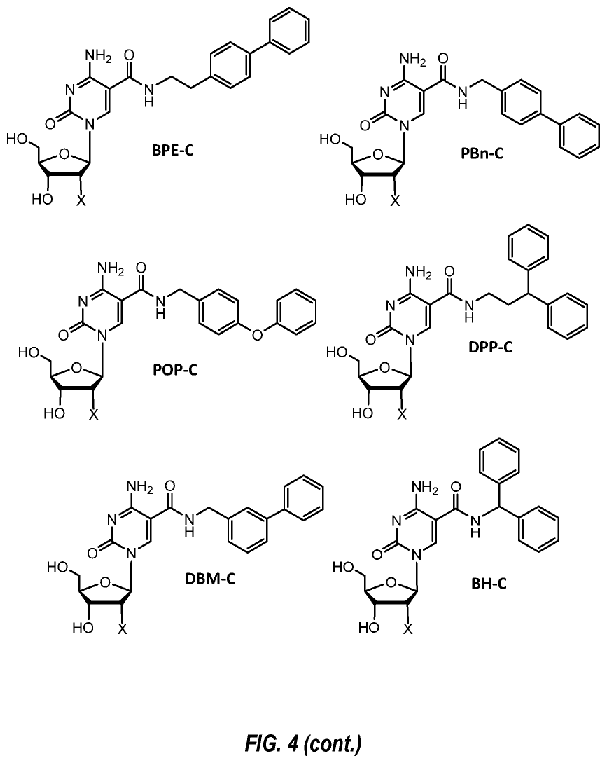

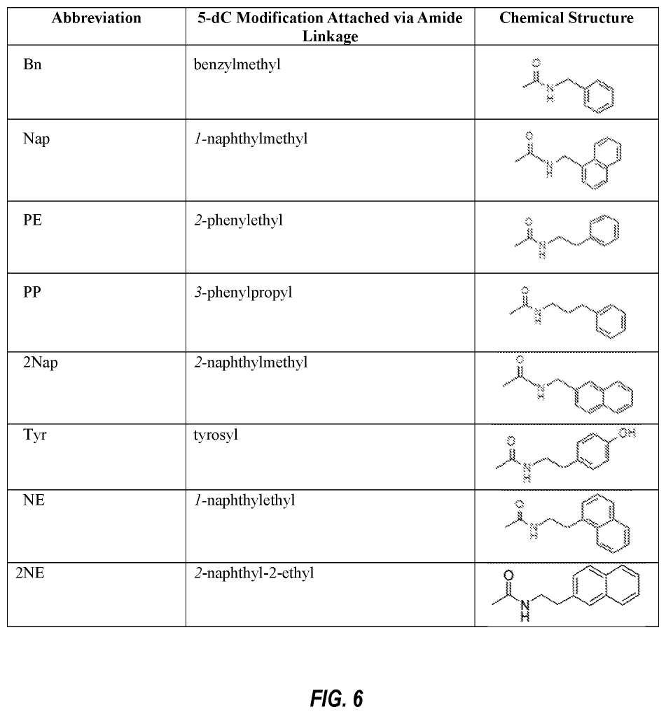

As used herein, the term “5-position modified cytidine” or “C-5 modified cytidine” refers to a cytidine with a modification at the C-5 position of the cytidine, e.g., as shown in FIG. 2 and FIG. 4. In some embodiments, the C5-modified cytidines, e.g., in their triphosphate form, are capable of being incorporated into an oligonucleotide by a polymerase (e.g., KOD DNA polymerase). Nonlimiting exemplary 5-position modified cytidines include those shown in FIG. 4. Nonlimiting exemplary C-5 modified cytidines include, but are not limited to, 5-[N-(4-phenylbenzyl)carboxamide]-2′-deoxycytidine (referred to as “PBndC” and shown in FIG. 4, where X═H); 5-[N-(4-phenylbenzyl)carboxamide]-2′-O-methylcytidine (referred to as “2′-OMe-PBn-C” and shown in FIG. 4, where X═OMe); 5-[N-(4-phenylbenzyl)carboxamide]-2′-fluorocytidine (referred to as “2′-F-PBn-C” and shown in FIG. 4, where X═F); 5-[N-(4-phenoxybenzyl)carboxamide]-2′-deoxycytidine (referred to as “POPdC” and shown in FIG. 4, where X═H); 5-[N-(4-phenoxybenzyl)carboxamide]-2′-O-methylcytidine (referred to as “2′-OMe-POP-C” and shown in FIG. 4, where X═OMe); 5-[N-(4-phenoxybenzyl)carboxamide]-2′-fluorocytidine (referred to as “2′-F-POP-C” and shown in FIG. 4, where X═F); 5-[N-(3,3-diphenylpropyl)carboxamide]-2′-deoxycytidine (referred to as “DPPdC” and shown in FIG. 4, where X═H); 5-[N-(3,3-diphenylpropyl)carboxamide]-2′-O-methylcytidine (referred to as “2′-OMe-DPP-C” and shown in FIG. 4, where X═OMe); 5-[N-(3,3-diphenylpropyl)carboxamide]-2′-fluorocytidine (referred to as “2′-F-DPP-C” and shown in FIG. 4, where X═F); 5-{N-[(1,1′-biphenyl)-4-yl)ethyl]carboxyamide}-2′-deoxycytidine (referred to as “BPEdC” and shown in FIG. 4, where X═H); 5-{N-[(1,1′-biphenyl)-4-yl)ethyl]carboxyamide}-2′-O-methylcytidine (referred to as “2′-OMe-BPE-C” and shown in FIG. 4, where X═OMe); 5-{N-[(1,1′-biphenyl)-4-yl)ethyl]carboxyamide}-2′-fluorocytidine (referred to as “2′-F-BPE-C” and shown in FIG. 4, where X═F); 5-[N-(3-phenylbenzyl)carboxamide]-2′-deoxycytidine (referred to as “DBMdC” and shown in FIG. 4, where X═H); 5-[N-(3-phenylbenzyl)carboxamide]-2′-O-methylcytidine (referred to as “2′-OMe-DBM-C” and shown in FIG. 4, where X═OMe); 5-[N-(3-phenylbenzyl)carboxamide]-2′-fluorocytidine (referred to as “2′-F-DBM-C” and shown in FIG. 4, where X═F); 5-[N-(3,3-diphenylmethyl)carboxamide]-2′-deoxycytidine (referred to as “BHdC” and shown in FIG. 4, where X═H); and 5-[N-(3,3-diphenylmethyl)carboxamide]-2′-O-methylcytidine (referred to as “2′-OMe-BH-C” and shown in FIG. 4, where X═OMe); and 5-[N-(3,3-diphenylmethyl)carboxamide]-2′-fluorocytidine (referred to as “2′-F-BH-C” and shown in FIG. 4, where X═F). Nonlimiting exemplary 5-position modified cytidines that may be further included, include those shown in FIG. 6. Nonlimiting exemplary 5-position modified cytidines that may be further included, include, but are not limited to, 5-(N-benzylcarboxamide)-2′-deoxycytidine (referred to as “BndC”); 5-(N-benzylcarboxamide)-2′-O-methylcytidine (referred to as “2′-OMe-Bn-C”); 5-(N-benzylcarboxamide)-2′-fluorocytidine (referred to as “2′-F-Bn-C”); 5-(N-2-phenylethylcarboxamide)-2′-deoxycytidine (referred to as “PEdC”); 5-(N-2-phenylethylcarboxamide)-2′-O-methylcytidine (referred to as “2′-OMe-PE-C”); 5-(N-2-phenylethylcarboxamide)-2′-fluorocytidine (referred to as “2′-F-PE-C”); 5-(N-3-phenylpropylcarboxamide)-2′-deoxycytidine (referred to as “PPdC”); 5-(N-3-phenylpropylcarboxamide)-2′-O-methylcytidine (referred to as “2′-OMe-PP-C”); 5-(N-3-phenylpropylcarboxamide)-2′-fluorocytidine (referred to as “2′-F-PP-C”); 5-(N-1-naphthylmethylcarboxamide)-2′-deoxycytidine (referred to as “NapdC”); 5-(N-1-naphthylmethylcarboxamide)-2′-O-methylcytidine (referred to as “2′-OMe-Nap-C”); 5-(N-1-naphthylmethylcarboxamide)-2′-fluorocytidine (referred to as “2′-F-Nap-C”); 5-(N-2-naphthylmethylcarboxamide)-2′-deoxycytidine (referred to as “2NapdC”); 5-(N-2-naphthylmethylcarboxamide)-2′-O-methylcytidine (referred to as “2′-OMe-2Nap-C”); 5-(N-2-naphthylmethylcarboxamide)-2′-fluorocytidine (referred to as “2′-F-2Nap-C”); 5-(N-1-naphthyl-2-ethylcarboxamide)-2′-deoxycytidine (referred to as “NEdC”); 5-(N-1-naphthyl-2-ethylcarboxamide)-2′-O-methylcytidine (referred to as “2′-OMe-NE-C”); 5-(N-1-naphthyl-2-ethylcarboxamide)-2′-fluorocytidine (referred to as “2′-F-NE-C”); 5-(N-2-naphthyl-2-ethylcarboxamide)-2′-deoxycytidine (referred to as “2NEdC”); 5-(N-2-naphthyl-2-ethylcarboxamide)-2′-O-methylcytidine (referred to as “2′-OMe-2NE-C”); 5-(N-2-naphthyl-2-ethylcarboxamide)-2′-fluorocytidine (referred to as “2′-F-2NE-C”); 5-(N-tyrosylcarboxamide)-2′-deoxycytidine (referred to as “TyrdC”); 5-(N-tyrosylcarboxamide)-2′-O-methylcytidine (referred to as “2′-OMe-Tyr-C”); and 5-(N-tyrosylcarboxamide)-2′-fluorocytidine (referred to as “2′-F-Tyr-C”).

In some embodiments, the C5-modified cytidines, e.g., in their triphosphate form, are capable of being incorporated into an oligonucleotide by a polymerase (e.g., KOD DNA polymerase).

Chemical modifications of the C-5 modified cytidines described herein can also be combined with, singly or in any combination, 2′-position sugar modifications (for example, 2′-O-methyl or 2′-fluoro), modifications at exocyclic amines, and substitution of 4-thiocytidine and the like.

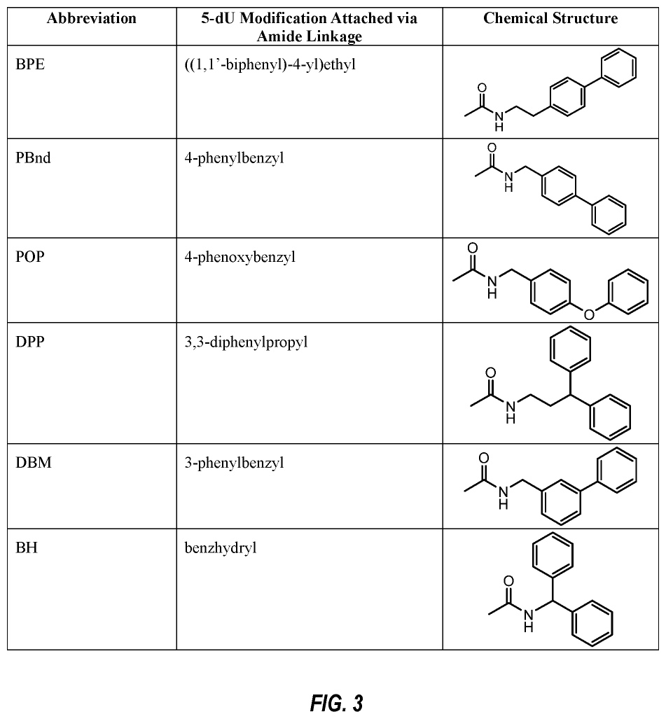

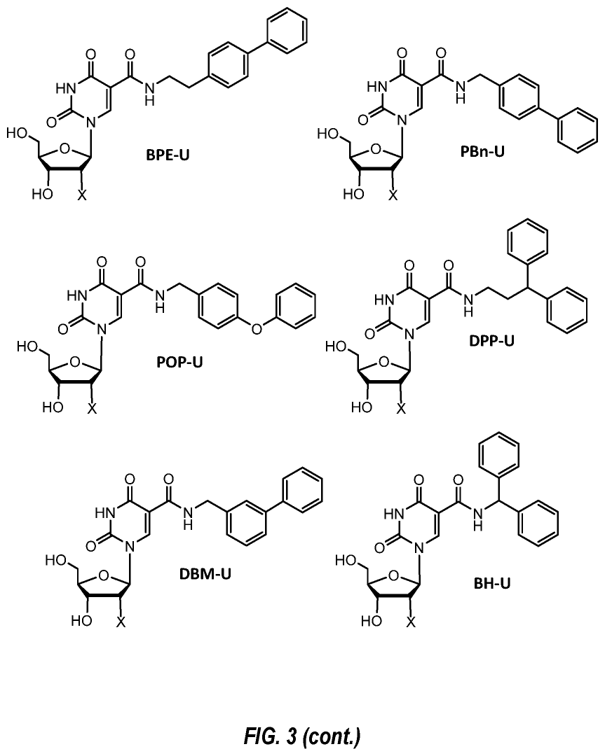

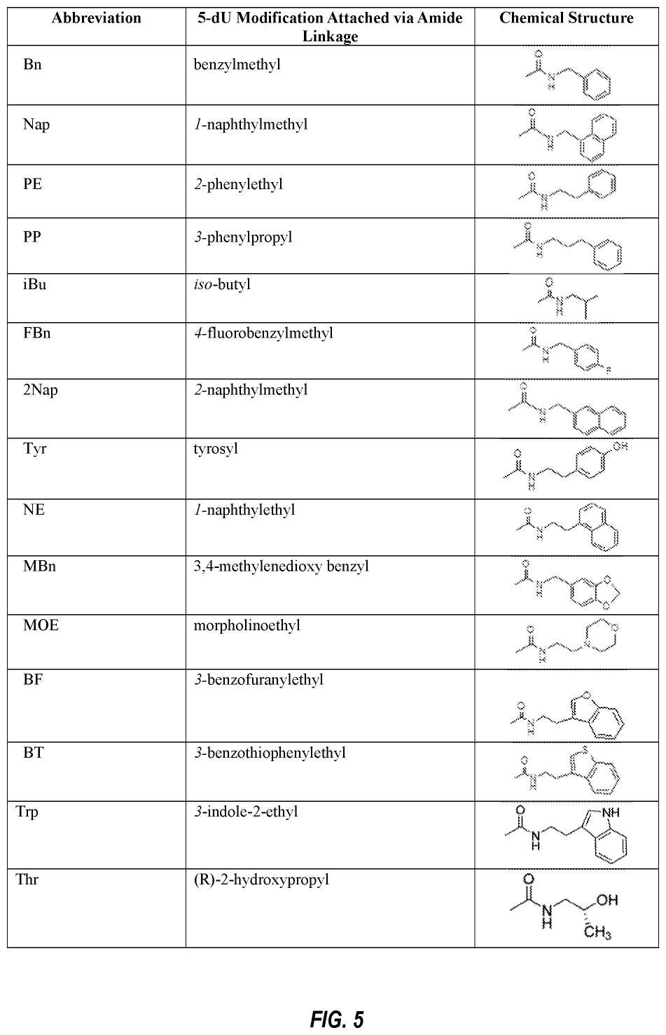

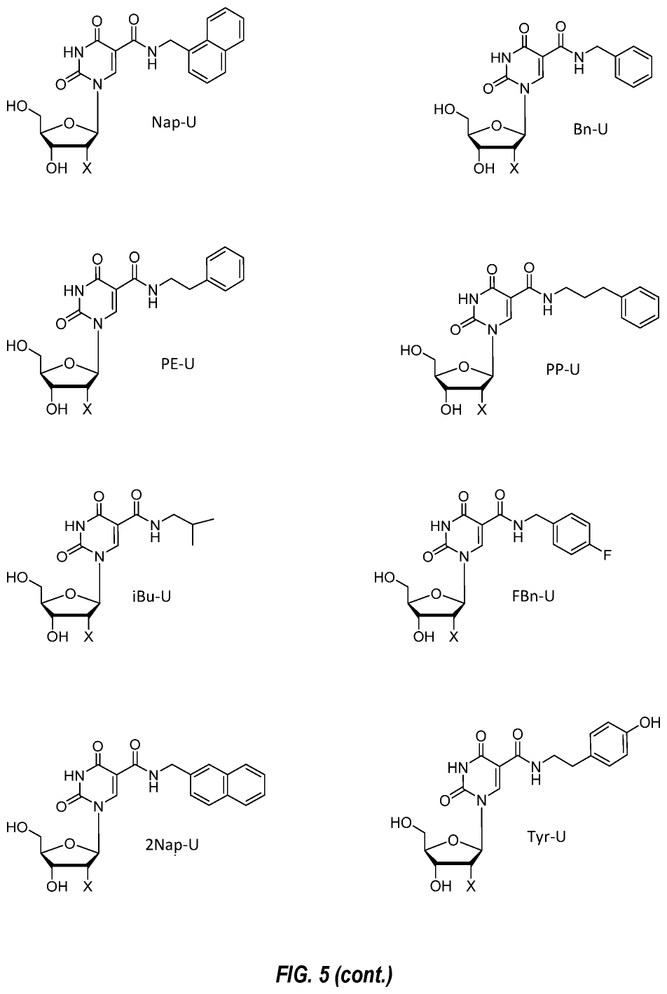

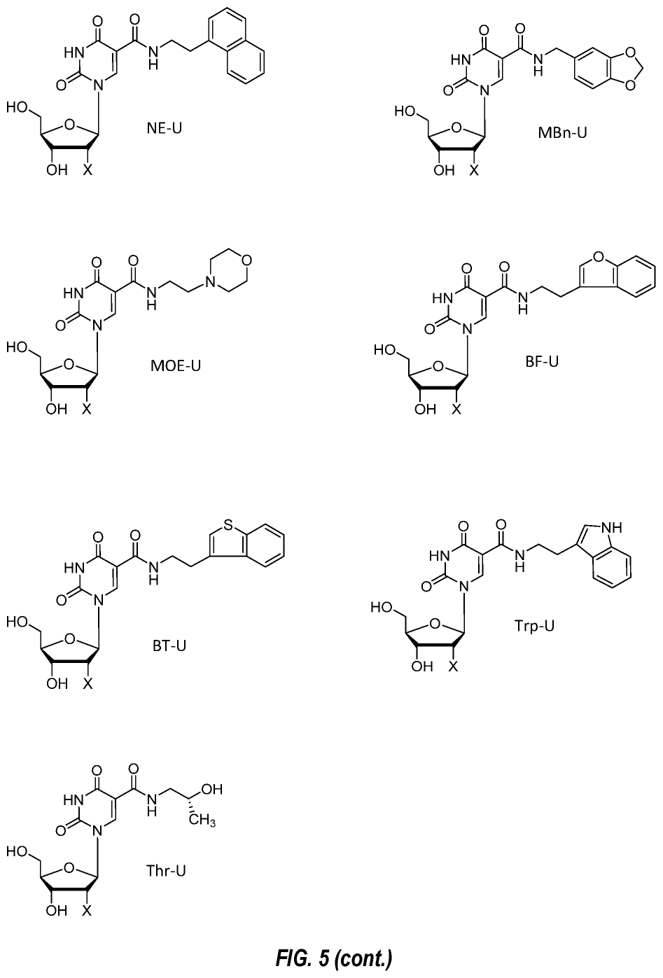

As used herein, the term “C-5 modified uridine” or “5-position modified uridine” refers to a uridine with a modification at the C-5 position of the uridine, e.g., as shown in FIG. 2 and FIG. 3. In some embodiments, the C5-modified uridines, e.g., in their triphosphate form, are capable of being incorporated into an oligonucleotide by a polymerase (e.g., KOD DNA polymerase). Nonlimiting exemplary 5-position modified uridines include those shown in FIG. 3. Nonlimiting exemplary 5-position modified uridines include, but are not limited to,
5-[N-(4-phenylbenzyl)carboxamide]-2′-deoxyuridine (PBndU),
5-[N-(4-phenylbenzyl)carboxamide]-2′-O-methyluridine (2′-OMe-PBn-U),
5-[N-(4-phenylbenzyl)carboxamide]-2′-fluorouridine (2′-F-PBn-U),
5-[N-(4-phenoxybenzyl)carboxamide]-2′-deoxyuridine (POPdU),
5-[N-(4-phenoxybenzyl)carboxamide]-2′-O-methyluridine (2′-OMe-POP-U),
5-[N-(4-phenoxybenzyl)carboxamide]-2′-fluorouridine (2′-F-POP-U),
5-[N-(3,3-diphenylpropyl)carboxamide]-2′-deoxyuridine (DPPdU),
5-[N-(3,3-diphenylpropyl)carboxamide]-2′-O-methyluridine (2′-OMe-DPP-U),
5-[N-(3,3-diphenylpropyl)carboxamide]-2′-fluorouridine (2′-F-DPP-U),
5-{N-[(1,1′-biphenyl)-4-yl)ethyl]carboxyamide}-2′-deoxyuridine (BPEdU),
5-{N-[(1,1′-biphenyl)-4-yl)ethyl]carboxyamide}-2′-O-methyluridine (2′-OMe-BPE-U),
5-{N-[(1,1′-biphenyl)-4-yl)ethyl]carboxyamide}-2′-fluorouridine (2′-F-BPE-U),
5-[N-(3-phenylbenzyl)carboxamide]-2′-deoxyuridine (DBMdU),
5-[N-(3-phenylbenzyl)carboxamide]-2′-O-methyluridine (2′-OMe-DBM-U),
5-[N-(3-phenylbenzyl)carboxamide]-2′-fluorouridine (2′-F-DBM-U),
5-[N-(3,3-diphenylmethyl)carboxamide]-2′-deoxyuridine (BHdU),
5-[N-(3,3-diphenylmethyl)carboxamide]-2′-O-methyluridine (2′-OMe-BH-U),
5-[N-(3,3-diphenylmethyl)carboxamide]-2′-fluorouridine (2′-F-BH-U).
   Nonlimiting exemplary 5-position modified uridines that may be further included, include those shown in FIG. 5.

Nonlimiting exemplary 5-position modified uridines that may be further included, include, but are not limited to, 5-(N-benzylcarboxamide)-2′-deoxyuridine (BndU), 5-(N-benzylcarboxamide)-2′-O-methyluridine (2′-OMe-Bn-U), 5-(N-benzylcarboxamide)-2′-fluorouridine (2′-F-Bn-U), 5-(N-phenethylcarboxamide)-2′-deoxyuridine (PEdU), 5-(N-phenethylcarboxamide)-2′-O-methyluridine (2′-OMe-PE-U), 5-(N-phenethylcarboxamide)-2′-fluorouridine (2′-F-PE-U), 5-(N-thiophenylmethylcarboxamide)-2′-deoxyuridine (ThdU), 5-(N-thiophenylmethylcarboxamide)-2′-O-methyluridine (2′-OMe-Th-U), 5-(N-thiophenylmethylcarboxamide)-2′-fluorouridine (2′-F-Th-U), 5-(N-isobutylcarboxamide)-2′-deoxyuridine (iBudU), 5-(N-isobutylcarboxamide)-2′-O-methyluridine (2′-OMe-iBu-U), 5-(N-isobutylcarboxamide)-2′-fluorouridine (2′-F-iBu-U), 5-(N-tyrosylcarboxamide)-2′-deoxyuridine (TyrdU), 5-(N-tyrosylcarboxamide)-2′-O-methyluridine (2′-OMe-Tyr-U), 5-(N-tyrosylcarboxamide)-2′-fluorouridine (2′-F-Tyr-U), 5-(N-3,4-methylenedioxybenzylcarboxamide)-2′-deoxyuridine (MBndU), 5-(N-3,4-methylenedioxybenzylcarboxamide)-2′-O-methyluridine (2′-OMe-MBn-U), 5-(N-3,4-methylenedioxybenzylcarboxamide)-2′-fluorouridine (2′-F-MBn-U), 5-(N-4-fluorobenzylcarboxamide)-2′-deoxyuridine (FBndU), 5-(N-4-fluorobenzylcarboxamide)-2′-O-methyluridine (2′-OMe-FBn-U), 5-(N-4-fluorobenzylcarboxamide)-2′-fluorouridine (2′-F-FBn-U), 5-(N-3-phenylpropylcarboxamide)-2′-deoxyuridine (PPdU), 5-(N-3-phenylpropylcarboxamide)-2′-O-methyluridine (2′-OMe-PP-U), 5-(N-3-phenylpropylcarboxamide)-2′-fluorouridine (2′-F-PP-U), 5-(N-imidizolylethylcarboxamide)-2′-deoxyuridine (ImdU), 5-(N-imidizolylethylcarboxamide)-2′-O-methyluridine (2′-OMe-Im-U), 5-(N-imidizolylethylcarboxamide)-2′-fluorouridine (2′-F-Im-U), 5-(N-tryptaminocarboxamide)-2′-deoxyuridine (TrpdU), 5-(N-tryptaminocarboxamide)-2′-O-methyluridine (2′-OMe-Trp-U), 5-(N-tryptaminocarboxamide)-2′-fluorouridine (2′-F-Trp-U), 5-(N—R-threoninylcarboxamide)-2′-deoxyuridine (ThrdU), 5-(N—R-threoninylcarboxamide)-2′-O-methyluridine (2′-OMe-Thr-U), 5-(N—R-threoninylcarboxamide)-2′-fluorouridine (2′-F-Thr-U), 5-(N-[1-(3-trimethylamonium) propyl]carboxamide)-2′-deoxyuridine chloride, 5-(N-[1-(3-trimethylamonium) propyl]carboxamide)-2′-O-methyluridine chloride, 5-(N-[1-(3-trimethylamonium) propyl]carboxamide)-2′-fluorouridine chloride, 5-(N-naphthylmethylcarboxamide)-2′-deoxyuridine (NapdU), 5-(N-naphthylmethylcarboxamide)-2′-O-methyluridine (2′-OMe-Nap-U), 5-(N-naphthylmethylcarboxamide)-2′-fluorouridine (2′-F-Nap-U), 5-(N-[1-(2,3-dihydroxypropyl)]carboxamide)-2′-deoxyuridine), 5-(N-[1-(2,3-dihydroxypropyl)]carboxamide)-2′-O-methyluridine), 5-(N-[1-(2,3-dihydroxypropyl)]carboxamide)-2′-fluorouridine), 5-(N-2-naphthylmethylcarboxamide)-2′-deoxyuridine (2NapdU), 5-(N-2-naphthylmethylcarboxamide)-2′-O-methyluridine (2′-OMe-2Nap-U), 5-(N-2-naphthylmethylcarboxamide)-2′-fluorouridine (2′-F-2Nap-U), 5-(N-1-naphthylethylcarboxamide)-2′-deoxyuridine (NEdU), 5-(N-1-naphthylethylcarboxamide)-2′-O-methyluridine (2′-OMe-NE-U), 5-(N-1-naphthylethylcarboxamide)-2′-fluorouridine (2′-F-NE-U), 5-(N-2-naphthylethylcarboxamide)-2′-deoxyuridine (2NEdU), 5-(N-2-naphthylethylcarboxamide)-2′-O-methyluridine (2′-OMe-2NE-U), 5-(N-2-naphthylethylcarboxamide)-2′-fluorouridine (2′-F-2NE-U), 5-(N-3-benzofuranylethylcarboxamide)-2′-deoxyuridine (BFdU), 5-(N-3-benzofuranylethylcarboxamide)-2′-O-methyluridine (2′-OMe-BF-U), 5-(N-3-benzofuranylethylcarboxamide)-2′-fluorouridine (2′-F—BF-U), 5-(N-3-benzothiophenylethylcarboxamide)-2′-deoxyuridine (BTdU), 5-(N-3-benzothiophenylethylcarboxamide)-2′-O-methyluridine (2′-OMe-BT-U), 5-(N-3-benzothiophenylethylcarboxamide)-2′-fluorouridine (2′-F-BT-U).

As used herein, the terms “modify,” “modified,” “modification,” and any variations thereof, when used in reference to an oligonucleotide, means that at least one of the four constituent nucleotide bases (i.e., A, G, T/U, and C) of the oligonucleotide is an analog or ester of a naturally occurring nucleotide. In some embodiments, the modified nucleotide confers nuclease resistance to the oligonucleotide. Additional modifications can include backbone modifications, methylations, unusual base-pairing combinations such as the isobases isocytidine and isoguanidine, and the like. Modifications can also include 3′ and 5′ modifications, such as capping. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers in some embodiments ranging from about 10 to about 80 kDa, PEG polymers in another embodiment ranging from about 20 to about 60 kDa, or other hydrophilic or hydrophobic biological or synthetic polymers.

As used herein, “nucleic acid,” “oligonucleotide,” and “polynucleotide” are used interchangeably to refer to a polymer of nucleotides and include DNA, RNA, DNA/RNA hybrids and modifications of these kinds of nucleic acids, oligonucleotides and polynucleotides, wherein the attachment of various entities or moieties to the nucleotide units at any position are included. The terms “polynucleotide,” “oligonucleotide,” and “nucleic acid” include double- or single-stranded molecules as well as triple-helical molecules. Nucleic acid, oligonucleotide, and polynucleotide are broader terms than the term aptamer and, thus, the terms nucleic acid, oligonucleotide, and polynucleotide include polymers of nucleotides that are aptamers but the terms nucleic acid, oligonucleotide, and polynucleotide are not limited to aptamers.

Polynucleotides can also contain analogous forms of ribose or deoxyribose sugars that are generally known in the art, including 2′-O-methyl, 2′-O-allyl, 2′-O-ethyl, 2′-O-propyl, 2′-O-CH₂CH₂OCH₃, 2′-fluoro, 2′-NH₂ or 2′-azido, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. As noted herein, one or more phosphodiester linkages may be replaced by alternative linking groups. These alternative linking groups include embodiments wherein phosphate is replaced by P(O)S (“thioate”), P(S)S (“dithioate”), (O)NRO^{X} ₂ (“amidate”), P(O) R^{X}, P(O)OR^{X}′, CO or CH₂ (“formacetal”), in which each R^{X} or R^{X}′ are independently H or substituted or unsubstituted alkyl (C1-C20) optionally containing an ether (—O—) linkage, aryl, alkenyl, cycloalky, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

Polynucleotides can also contain analogous forms of carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside.

If present, a modification to the nucleotide structure can be imparted before or after assembly of a polymer. A sequence of nucleotides can be interrupted by non-nucleotide components. A polynucleotide can be further modified after polymerization, such as by conjugation with a labeling component.

As used herein, the term “at least one nucleotide” when referring to modifications of a nucleic acid, refers to one, several, or all nucleotides in the nucleic acid, indicating that any or all occurrences of any or all of A, C, T, G or U in a nucleic acid may be modified or not.

As used herein, “nucleic acid ligand,” “aptamer,” “SOMAmer,” and “clone” are used interchangeably to refer to a non-naturally occurring nucleic acid that has a desirable action on a target molecule. A desirable action includes, but is not limited to, binding of the target, catalytically changing the target, reacting with the target in a way that modifies or alters the target or the functional activity of the target, covalently attaching to the target (as in a suicide inhibitor), and facilitating the reaction between the target and another molecule. In some embodiments, the action is specific binding affinity for a target molecule, such target molecule being a three dimensional chemical structure other than a polynucleotide that binds to the aptamer through a mechanism which is independent of Watson/Crick base pairing or triple helix formation, wherein the aptamer is not a nucleic acid having the known physiological function of being bound by the target molecule. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target generally with a much higher degree of affinity than it binds to other, non-target, components in a mixture or sample. An “aptamer,” “SOMAmer,” or “nucleic acid ligand” is a set of copies of one type or species of nucleic acid molecule that has a particular nucleotide sequence. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions.

In some embodiments, the aptamers are prepared using a SELEX process as described herein, or known in the art.

As used herein, a “SOMAmer” or Slow Off-Rate Modified Aptamer refers to an aptamer having improved off-rate characteristics. SOMAmers can be generated using the improved SELEX methods described in U.S. Pat. No. 7,947,447, entitled “Method for Generating Aptamers with Improved Off-Rates.”

As used herein, an aptamer comprising two different types of 5-position modified pyrimidines or C-5 modified pyrimidines may be referred to as “dual modified aptamers”, aptamers having “two modified bases”, aptamers having “two base modifications” or “two bases modified”, aptamer having “double modified bases”, all of which may be used interchangeably. A library of aptamers or aptamer library may also use the same terminology. Thus, in some embodiments, an aptamer comprises two different 5-position modified pyrimidines, which are selected from a BPEdU (or 2′-modified version thereof, such as a 2′-OMe-BPE-U) and a BPEdC (or 2′-modified version thereof, such as a 2′-OMe-BPE-C), a BPEdU (or 2′-modified version thereof, such as a 2′-OMe-BPE-U) and a PBndC (or 2′-modified version thereof, such as a 2′-OMe-PBn-C), a BPEdU (or 2′-modified version thereof, such as a 2′-OMe-BPE-U) and a POPdC (or 2′-modified version thereof, such as a 2′-OMe-POP-C), a BPEdU (or 2′-modified version thereof, such as a 2′-OMe-BPE-U) and a DPPdC (or 2′-modified version thereof, such as a 2′-OMe-DPP-C), a BPEdU (or 2′-modified version thereof, such as a 2′-OMe-BPE-U) and a DBMdC (or 2′-modified version thereof, such as a 2′-OMe-DBM-C), a BPEdU (or 2′-modified version thereof, such as a 2′-OMe-BPE-U) and a BHdC (or 2′-modified version thereof, such as a 2′-OMe-BH-C), a PBndU (or 2′-modified version thereof, such as a 2′-OMe-PBn-U) and a BPEdC (or 2′-modified version thereof, such as a 2′-OMe-BPE-C), a PBndU (or 2′-modified version thereof, such as a 2′-OMe-PBn-U) and a PBndC (or 2′-modified version thereof, such as a 2′-OMe-PBn-C), a PBndU (or 2′-modified version thereof, such as a 2′-OMe-PBn-U) and a POPdC (or 2′-modified version thereof, such as a 2′-OMe-POP-C), a PBndU (or 2′-modified version thereof, such as a 2′-OMe-PBn-U) and a DPPdC (or 2′-modified version thereof, such as a 2′-OMe-DPP-C), a POPdU (or 2′-modified version thereof, such as a 2′-OMe-POP-U) and a BPEdC (or 2′-modified version thereof, such as a 2′-OMe-BPE-C), a POPdU (or 2′-modified version thereof, such as a 2′-OMe-POP-U) and a PBndC (or 2′-modified version thereof, such as a 2′-OMe-PBn-C), a POPdU (or 2′-modified version thereof, such as a 2′-OMe-POP-U) and a POPdC (or 2′-modified version thereof, such as a 2′-OMe-POP-C), a POPdU (or 2′-modified version thereof, such as a 2′-OMe-POP-U) and a DPPdC (or 2′-modified version thereof, such as a 2′-OMe-DPP-C), a DPPdU (or 2′-modified version thereof, such as a 2′-OMe-DPP-U) and a BPEdC (or 2′-modified version thereof, such as a 2′-OMe-BPE-C), a DPPdU (or 2′-modified version thereof, such as a 2′-OMe-DPP-U) and a PBndC (or 2′-modified version thereof, such as a 2′-OMe-PBn-C), a DPPdU (or 2′-modified version thereof, such as a 2′-OMe-DPP-U) and a POPdC (or 2′-modified version thereof, such as a 2′-OMe-POP-C), a DPPdU (or 2′-modified version thereof, such as a 2′-OMe-DPP-U) and a DPPdC (or 2′-modified version thereof, such as a 2′-OMe-DPP-C), a DBMdU (or 2′-modified version thereof, such as a 2′-OMe-DBM-U) and a BPEdC (or 2′-modified version thereof, such as a 2′-OMe-BPE-C), a DBMdU (or 2′-modified version thereof, such as a 2′-OMe-DBM-U) and a PBndC (or 2′-modified version thereof, such as a 2′-OMe-PBn-C), a DBMdU (or 2′-modified version thereof, such as a 2′-OMe-DBM-U) and a POPdC (or 2′-modified version thereof, such as a 2′-OMe-POP-C), a DBMdU (or 2′-modified version thereof, such as a 2′-OMe-DBM-U) and a DPPdC (or 2′-modified version thereof, such as a 2′-OMe-DPP-C), a DBMdU (or 2′-modified version thereof, such as a 2′-OMe-DBM-U) and a DBMdC (or 2′-modified version thereof, such as a 2′-OMe-DBM-C), a DBMdU (or 2′-modified version thereof, such as a 2′-OMe-DBM-U) and a BHdC (or 2′-modified version thereof, such as a 2′-OMe-BH-C), a BHdU (or 2′-modified version thereof, such as a 2′-OMe-BH-U) and a BPEdC (or 2′-modified version thereof, such as a 2′-OMe-BPE-C), a BHdU (or 2′-modified version thereof, such as a 2′-OMe-BH-U) and a PBndC (or 2′-modified version thereof, such as a 2′-OMe-PBn-C), a BHdU (or 2′-modified version thereof, such as a 2′-OMe-BH-U) and a POPdC (or 2′-modified version thereof, such as a 2′-OMe-POP-C), a BHdU (or 2′-modified version thereof, such as a 2′-OMe-BH-U) and a DPPdC (or 2′-modified version thereof, such as a 2′-OMe-DPP-C), a BHdU (or 2′-modified version thereof, such as a 2′-OMe-BH-U) and a DBMdC (or 2′-modified version thereof, such as a 2′-OMe-DBM-C), a BHdU (or 2′-modified version thereof, such as a 2′-OMe-BH-U) and a BHdC (or 2′-modified version thereof, such as a 2′-OMe-BH-C). In some embodiments, an aptamer comprises two different 5-position modified pyrimidines, wherein the first 5-position modified pyrimidine is selected from a BPEdU (or 2′-modified version thereof, such as a 2′-OMe-BPE-U), a PBndU (or 2′-modified version thereof, such as a 2′-OMe-PBn-U), a POPdU (or 2′-modified version thereof, such as a 2′-OMe-POP-U), a DPPdU (or 2′-modified version thereof, such as a 2′-OMe-DPP-U), a BPEdC (or 2′-modified version thereof, such as a 2′-OMe-BPE-C), a PBndC (or 2′-modified version thereof, such as a 2′-OMe-PBn-C), a POPdC (or 2′-modified version thereof, such as a 2′-OMe-POP-C), a DPPdC (or 2′-modified version thereof, such as a 2′-OMe-DPP-C), and wherein the second 5-position modified pyrimidine is a different 5-position modified pyrimidine, which is selected from a NapdC (or 2′-modified version thereof, such as a 2′-OMe-Nap-C), a NapdU (or 2′-modified version thereof, such as a 2′-OMe-Nap-U), a PPdU (or 2′-modified version thereof, such as a 2′-OMe-PP-U), a MOEdU (or 2′-modified version thereof, such as a 2′-OMe-MOE-U), a TyrdU (or 2′-modified version thereof, such as a 2′-OMe-Tyr-U), a ThrdU (or 2′-modified version thereof, such as a 2′-OMe-Thr-U), a PPdC (or 2′-modified version thereof, such as a 2′-OMe-PP-C), a 2NapdU (or 2′-modified version thereof, such as a 2′-OMe-2Nap-U), a TrpdU (or 2′-modified version thereof, such as a 2′-OMe-Trp-U), a 2NapdC (or 2′-modified version thereof, such as a 2′-OMe-2Nap-C), a TyrdC (or 2′-modified version thereof, such as a 2′-OMe-Tyr-C). In some embodiments, an aptamer comprises at least one first modified uridine and/or thymidine or at least one first modified cytidine, wherein the at least one first modified uridine and/or thymidine or at least one first modified cytidine is modified at the 5-position with a moiety comprising two phenyl groups covalently attached to one another. In some embodiments, an aptamer comprises at least one second modified uridine and/or thymidine and at least one second modified cytidine, wherein the at least one second modified uridine and/or thymidine is modified at the 5-position with a moiety selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety, and wherein the at least one second modified cytidine is modified at the 5-position with a moiety selected from a naphthyl moiety, a tyrosyl moiety, and a benzyl moiety. In certain embodiments, the moiety is covalently linked to the 5-position of the base via a linker comprising a group selected from an amide linker, a carbonyl linker, an ester linker, a urea linker, a carbamate linker, a guanidine linker, an amidine linker, a sulfoxide linker, and a sulfone linker.

As used herein, an aptamer comprising a single type of 5-position modified pyrimidine or C-5 modified pyrimidine may be referred to as “single modified aptamers”, aptamers having a “single modified base”, aptamers having a “single base modification” or “single bases modified”, all of which may be used interchangeably. A library of aptamers or aptamer library may also use the same terminology. As used herein, “protein” is used synonymously with “peptide,” “polypeptide,” or “peptide fragment.” A “purified” polypeptide, protein, peptide, or peptide fragment is substantially free of cellular material or other contaminating proteins from the cell, tissue, or cell-free source from which the amino acid sequence is obtained, or substantially free from chemical precursors or other chemicals when chemically synthesized.

In certain embodiments, at least 10%, 15%, 20%, 25%, 30%, 35%, 40%, 45%, 50%, 55%, 60%, 65%, 70%, 75%, 80%, 85%, 90%, 95%, or 100% of the uracils of the aptamer are modified at the 5-position. In certain embodiments, at least 10%, 15%, 20%, 25%, 30%, 35%, 40%, 45%, 50%, 55%, 60%, 65%, 70%, 75%, 80%, 85%, 90%, 95%, or 100% of the cytosine of the aptamer are modified at the 5-position.

### Modified Nucleotides

In certain embodiments, the disclosure provides oligonucleotides, such as aptamers, which comprise 5-position modified pyrimidines.

In some embodiments, the disclosure provides compounds comprising a 5-position modified pyrimidine nucleoside, or a salt thereof, wherein the 5-position modified pyrimidine is substituted with a moiety comprising two phenyl groups covalently attached to one another by a first linker, and wherein the moiety is covalently linked to the 5-position of the pyrimidine by a second linker.

In some embodiments, the first linker comprises at least one atom selected from a carbon and oxygen or is a bond.

In some embodiments, the 5-position modified pyrimidine comprises a moiety at the 5-position selected from a phenylbenzyl moiety, a phenoxybenzyl moiety, and a diphenylmethyl moiety.

In some embodiments, the second linker comprises a group selected from an amide linker, a carbonyl linker, a propynyl linker, an alkyne linker, an ester linker, a urea linker, a carbamate linker, a guanidine linker, an amidine linker, a sulfoxide linker, and a sulfone linker.

In some embodiments, the second linker comprises an amide linker. In some embodiments, the amide linker further comprises one or more carbon atoms or 1, 2, 3, 4, 5, 6, 7, 8, 9 or 10 carbon atoms.

In some embodiments, the compound comprises a 5-position modified uridine.

In some embodiments, the compound comprises a 5-position modified cytidine.

In some embodiments, the disclosure provides oligonucleotides comprising the structure of Formula IA or Formula IB:

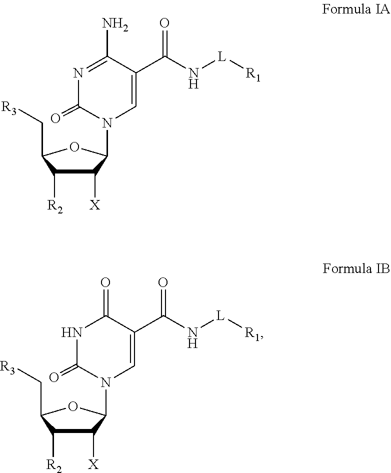

or a salt of either one of these.

In some embodiments, each L is independently a —(CH₂)ₙ—, wherein n is 0, 1, 2, 3, 4, 5, 6, 7, 8, 9, or 10.

In some embodiments, each R₁ is independently selected from the group consisting of

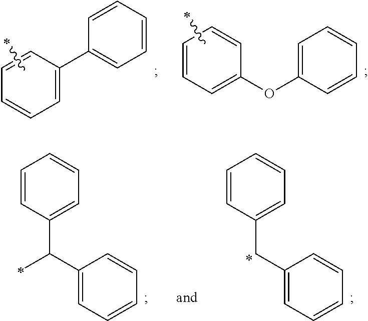

wherein * denotes the point of attachment of the R₁ group to the L group.

In some embodiments, each X is independently selected from the group consisting of —H, —OH, —OMe, —O-allyl, —O-ethyl, —O-propyl, —OCH₂CH₂OCH₃, -fluoro, tert-butyldimethylsilyloxy, —NH₂, and -azido.

In some embodiments, each R₂ is independently selected from the group consisting of —OH; -acetyl; —OBz; —OP(N(CH₂CH₃)₂)(OCH₂CH₂CN), —OP(N(Rₓ)₂)(OCH₂CH₂CN), wherein each Rₓ is independently (C₁₋₆)alkyl; tert-butyldimethylsilyloxy; —O-ss; —OR; —SR; —ZP(Z′)(Z″)—O—R; wherein ss is a solid support, Z, Z′, and Z″ are each independently selected from O and S, and R is an adjacent nucleotide.

In some embodiments, each R₃ is independently selected from the group consisting of —OH, —O-trityl, —O-4,4′-dimethoxytrityl, —O-triphosphate, —OR, —SR, —NH₂, —NHR, and —Z—P(Z′)(Z″)O—R, wherein Z, Z′, and Z″ are each independently selected from O and S, and R is an adjacent nucleotide.

In some embodiments, n is 1, 2, or 3.

In some embodiments, X is —H or —OMe.

In some embodiments, each R₁ is independently selected from the group consisting of

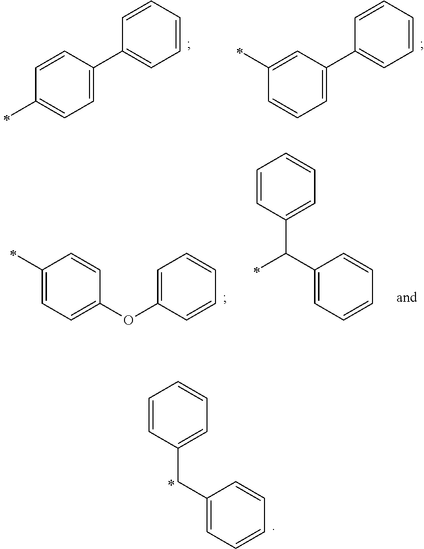

In some embodiments, the 5-position modified pyrimidine is selected from a BPEdU, a 2′-OMe-BPE-U, a 2′-F-BPE-U, a PBndU, a 2′-OMe-PBn-U, a 2′-F-PBn-U, a POPdU, a 2′-OMe-POP-U, a 2′-F-POP-U, a DPPdU, a 2′-OMe-DPP-U, a 2′-F-DPP-U, a DBMdU, a 2′-OMe-DBM-U, a 2′-F-DBM-U, a BHdU, a 2′-OMe-BH-U, a 2′-F-BH-U, a BPEdC, a 2′-OMe-BPE-C, a 2′-F-BPE-C, a PBndC, a 2′-OMe-PBn-C, a 2′-F-PBn-C, a POPdC, a 2′-OMe-POP-C, a 2′-F-POP-C, a DPPdC, a 2′-OMe-DPP-C, a 2′-F-DPP-C, a DBMdC, a 2′-OMe-DBM-C, a 2′-F-DBM-C, a BHdC, a 2′-OMe-BH-C, and a 2′-F-BH-C.

In some embodiments, the disclosure provides compound comprising the structure of Formula IIA or Formula IIB:

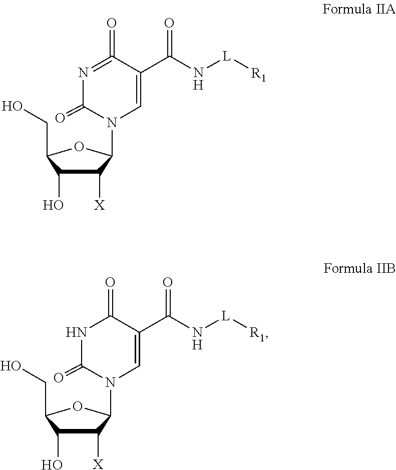

or a salt of either one of these.

In some embodiments, each L is independently a —(CH₂)ₙ—, wherein n is 0, 1, 2, 3, 4, 5, 6, 7, 8, 9, or 10.

In some embodiments, each R₁ is independently selected from the group consisting of

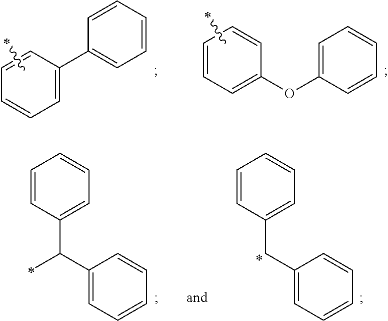

wherein * denotes the point of attachment of the R₁ group to the L group.

In some embodiments, each X is independently selected from the group consisting of —H, —OH, —OMe, —O-allyl, —O-ethyl, —O-propyl, —OCH₂CH₂OCH₃, -fluoro, tert-butyldimethylsilyloxy, —NH₂, and -azido.

In some embodiments, n is 1, 2, or 3.

In some embodiments, X is —H or —OMe.

In some embodiments, each R₁ is independently selected from the group consisting of

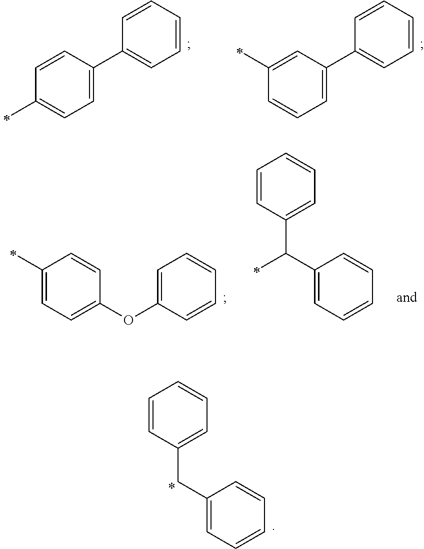

In some embodiments, the oligonucleotide comprises at least one modified pyrimidine as shown in FIG. 3 or FIG. 4, wherein each X is independently selected from —H, —OH, —OMe, —O-allyl, —F, —OEt, —OPr, —OCH₂CH₂OCH₃, NH₂ and -azido.

In some embodiments, the disclosure provides a compound selected from

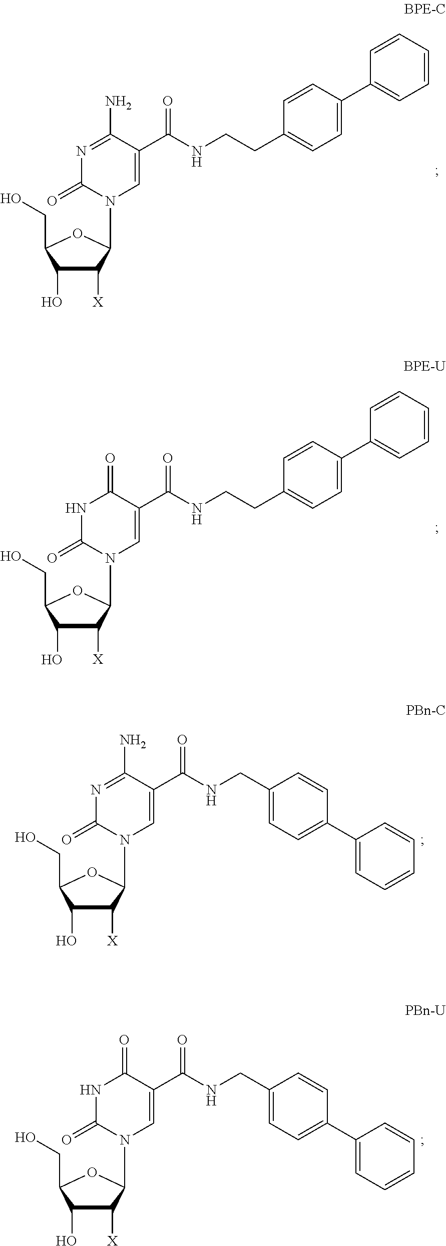

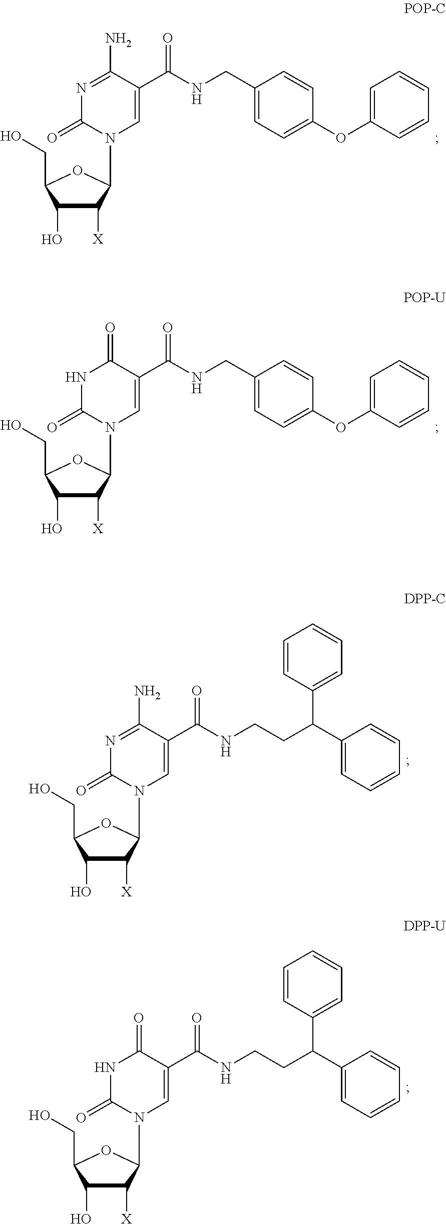

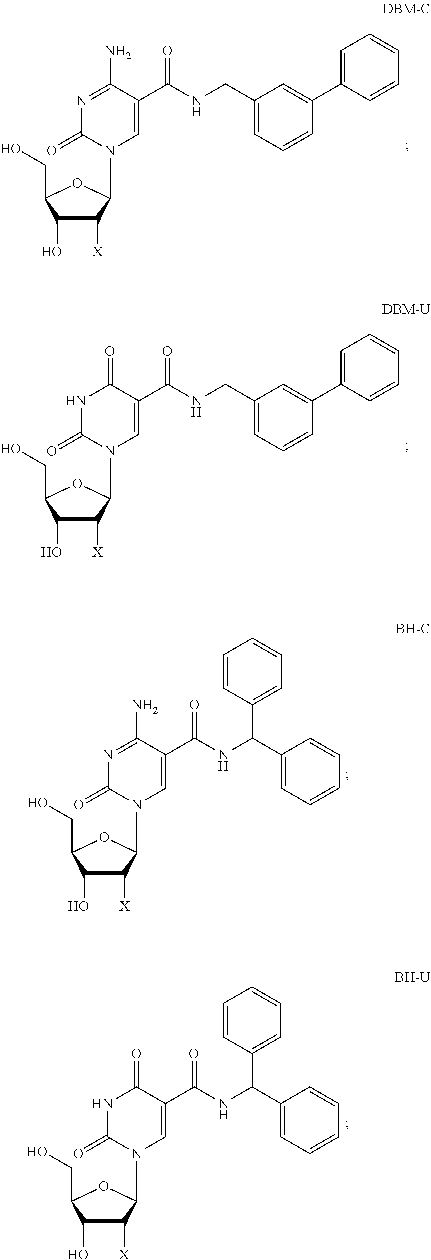

or a salt of any one of these.

In some embodiments, each X is independently selected from the group consisting of —H, —OH, —O-methyl, —O-allyl, —O-ethyl, —O-propyl, —OCH₂CH₂OCH₃, -fluoro, tert-butyldimethylsilyloxy, —NH₂, and -azido.

In some embodiments, X is —H or —OMe.

In some embodiments, a compound is provided, comprising the structure of Formula III, Formula IV, or Formula V:

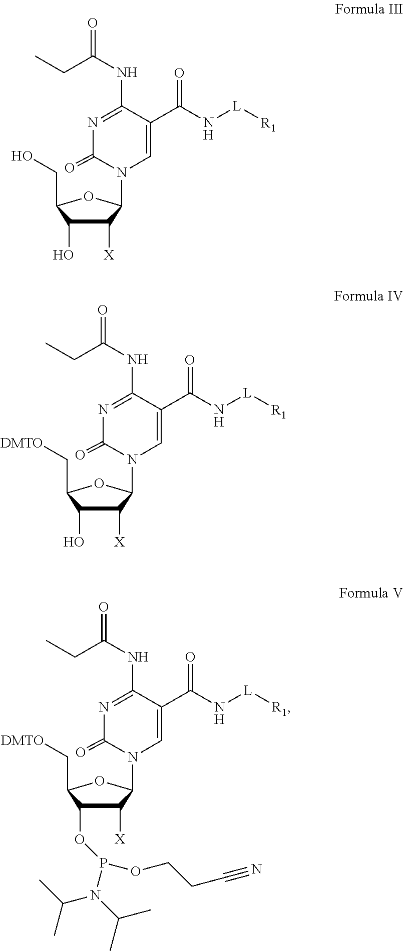

or a salt of any one of these.

In some embodiments, each L is independently a —(CH₂)ₙ—, wherein n is 0, 1, 2, 3, 4, 5, 6, 7, 8, 9, or 10.

In some embodiments, each R₁ is independently selected from the group consisting of

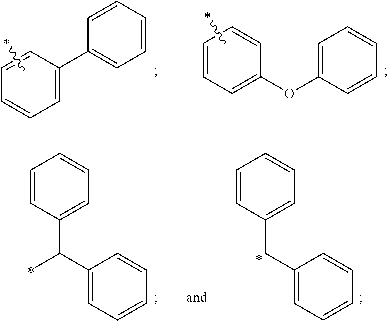

wherein * denotes the point of attachment of the R₁ group to the L group.

In some embodiments, each X is independently selected from the group consisting of —H, —OH, —OMe, —O-allyl, —O-ethyl, —O-propyl, —OCH₂CH₂OCH₃, -fluoro, tert-butyldimethylsilyloxy, —NH₂, and -azido.

In some embodiments, n is 1, 2, or 3.

In some embodiments, X is —H or —OMe.

In some embodiments, each R₁ is independently selected from the group consisting of

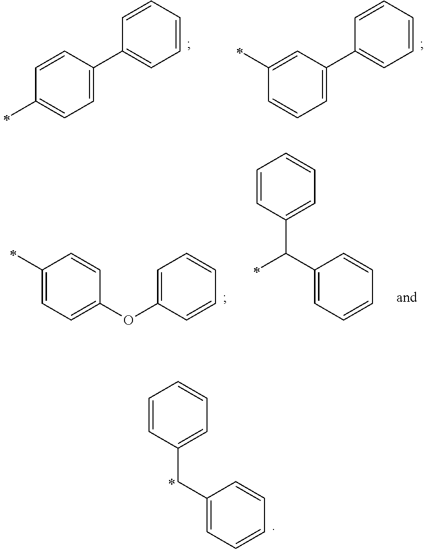

In any of the embodiments described herein, the oligonucleotide may be an aptamer. In some such embodiments, the oligonucleotide is an aptamer that specifically binds a target polypeptide.

### Preparation of Oligonucleotides

The automated synthesis of oligodeoxynucleosides is routine practice in many laboratories (see e.g., Matteucci, M. D. and Caruthers, M. H., (1990) J. Am. Chem. Soc., 103:3185-3191, the contents of which are hereby incorporated by reference in their entirety). Synthesis of oligoribonucleosides is also well known (see e.g. Scaringe, S. A., et al., (1990) Nucleic Acids Res. 18:5433-5441, the contents of which are hereby incorporated by reference in their entirety). As noted herein, the phosphoramidites are useful for incorporation of the modified nucleoside into an oligonucleotide by chemical synthesis, and the triphosphates are useful for incorporation of the modified nucleoside into an oligonucleotide by enzymatic synthesis. (See e.g., Vaught, J. D. et al. (2004) J. Am. Chem. Soc., 126:11231-11237; Vaught, J. V., et al. (2010) J. Am. Chem. Soc. 132, 4141-4151; Gait, M. J. “Oligonucleotide Synthesis a practical approach” (1984) IRL Press (Oxford, UK); Herdewijn, P. “Oligonucleotide Synthesis” (2005) (Humana Press, Totowa, N.J. (each of which is incorporated herein by reference in its entirety).

In some embodiments, the compounds provided herein may be used in standard phosphoramidite oligonucleotide synthesis methods, including automated methods using commercially available synthesizers.

In some embodiments, use of the compounds provided herein in oligonucleotide synthesis improves the yield of the desired oligonucleotide product.

### The SELEX Method

The terms “SELEX” and “SELEX process” are used interchangeably herein to refer generally to a combination of (1) the selection of nucleic acids that interact with a target molecule in a desirable manner, for example binding with high affinity to a protein, with (2) the amplification of those selected nucleic acids. The SELEX process can be used to identify aptamers with high affinity to a specific target molecule or biomarker.

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands.” The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides,” which describes oligonucleotides containing nucleotide derivatives chemically modified at the 5′- and 2′-positions of pyrimidines. U.S. Pat. No. 5,580,737, describes highly specific aptamers containing one or more nucleotides modified with 2′-amino (2′-NH₂), 2′-fluoro (2′-F), and/or 2′-O-methyl (2′-OMe). See also, U.S. Patent Application Publication No. 20090098549, entitled “SELEX and PHOTOSELEX,” which describes nucleic acid libraries having expanded physical and chemical properties and their use in SELEX and photoSELEX.

SELEX can also be used to identify aptamers that have desirable off-rate characteristics. See U.S. Pat. No. 7,947,447, entitled “Method for Generating Aptamers with Improved Off-Rates,” which is incorporated herein by reference in its entirety, describes improved SELEX methods for generating aptamers that can bind to target molecules. Methods for producing aptamers and photoaptamers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates dissociate and do not reform, while complexes with slow dissociation rates remain intact. Additionally, the methods include the use of modified nucleotides in the production of candidate nucleic acid mixtures to generate aptamers with improved off-rate performance (see U.S. Pat. No. 8,409,795, entitled “SELEX and PhotoSELEX”). (See also U.S. Pat. No. 7,855,054 and U.S. Patent Publication No. 20070166740). Each of these applications is incorporated herein by reference in its entirety.

“Target” or “target molecule” or “target” refers herein to any compound upon which a nucleic acid can act in a desirable manner. A target molecule can be a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell, tissue, any portion or fragment of any of the foregoing, etc., without limitation. Virtually any chemical or biological effector may be a suitable target. Molecules of any size can serve as targets. A target can also be modified in certain ways to enhance the likelihood or strength of an interaction between the target and the nucleic acid. A target can also include any minor variation of a particular compound or molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule” or “target” is a set of copies of one type or species of molecule or multimolecular structure that is capable of binding to an aptamer. “Target molecules” or “targets” refer to more than one such set of molecules. Embodiments of the SELEX process in which the target is a peptide are described in U.S. Pat. No. 6,376,190, entitled “Modified SELEX Processes Without Purified Protein.” In some embodiments, a target is a protein.

As used herein, “competitor molecule” and “competitor” are used interchangeably to refer to any molecule that can form a non-specific complex with a non-target molecule. In this context, non-target molecules include free aptamers, where, for example, a competitor can be used to inhibit the aptamer from binding (rebinding), non-specifically, to another non-target molecule. A “competitor molecule” or “competitor” is a set of copies of one type or species of molecule. “Competitor molecules” or “competitors” refer to more than one such set of molecules. Competitor molecules include, but are not limited to oligonucleotides, polyanions (e.g., heparin, herring sperm DNA, salmon sperm DNA, tRNA, dextran sulfate, polydextran, abasic phosphodiester polymers, dNTPs, and pyrophosphate). In various embodiments, a combination of one or more competitor can be used.

As used herein, “non-specific complex” refers to a non-covalent association between two or more molecules other than an aptamer and its target molecule. A non-specific complex represents an interaction between classes of molecules. Non-specific complexes include complexes formed between an aptamer and a non-target molecule, a competitor and a non-target molecule, a competitor and a target molecule, and a target molecule and a non-target molecule.

As used herein, the term “slow off-rate enrichment process” refers to a process of altering the relative concentrations of certain components of a candidate mixture such that the relative concentration of aptamer affinity complexes having slow dissociation rates is increased relative to the concentration of aptamer affinity complexes having faster, less desirable dissociation rates. In some embodiments, the slow off-rate enrichment process is a solution-based slow off-rate enrichment process. Accordingly, a solution-based slow off-rate enrichment process takes place in solution, such that neither the target nor the nucleic acids forming the aptamer affinity complexes in the mixture are immobilized on a solid support during the slow off-rate enrichment process. In various embodiments, the slow-off rate enrichment process can include one or more steps, including the addition of and incubation with a competitor molecule, dilution of the mixture, or a combination of these (e.g., dilution of the mixture in the presence of a competitor molecule). Because the effect of an slow off-rate enrichment process generally depends upon the differing dissociation rates of different aptamer affinity complexes (i.e., aptamer affinity complexes formed between the target molecule and different nucleic acids in the candidate mixture), the duration of the slow off-rate enrichment process is selected so as to retain a high proportion of aptamer affinity complexes having slow dissociation rates while substantially reducing the number of aptamer affinity complexes having fast dissociation rates. The slow off-rate enrichment process may be used in one or more cycles during the SELEX process. When dilution and the addition of a competitor are used in combination, they may be performed simultaneously or sequentially, in any order. The slow-off rate enrichment process can be used when the total target (protein) concentration in the mixture is low. In some embodiments, when the slow off-rate enrichment process includes dilution, the mixture can be diluted as much as is practical, keeping in mind that the aptamer retained nucleic acids are recovered for subsequent rounds in the SELEX process. In some embodiments, the slow off-rate enrichment process includes the use of a competitor as well as dilution, permitting the mixture to be diluted less than might be necessary without the use of a competitor.

In some embodiments, the slow off-rate enrichment process includes the addition of a competitor, and the competitor is a polyanion (e.g., heparin or dextran sulfate (dextran)). Heparin or dextran have been used in the identification of specific aptamers in prior SELEX selections. In such methods, however, heparin or dextran is present during the equilibration step in which the target and aptamer bind to form complexes. In such methods, as the concentration of heparin or dextran increases, the ratio of high affinity target/aptamer complexes to low affinity target/aptamer complexes increases. However, a high concentration of heparin or dextran can reduce the number of high affinity target/aptamer complexes at equilibrium due to competition for target binding between the nucleic acid and the competitor. By contrast, in some embodiments, the methods add the competitor after the target/aptamer complexes have been allowed to form and therefor does not affect the number of complexes formed. Addition of competitor after equilibrium binding has occurred between target and aptamer creates a non-equilibrium state that evolves in time to a new equilibrium with fewer target/aptamer complexes. Trapping target/aptamer complexes before the new equilibrium has been reached enriches the sample for slow off-rate aptamers since fast off-rate complexes will dissociate first.

In other embodiments, a polyanionic competitor (e.g., dextran sulfate or another polyanionic material) is used in the slow off-rate enrichment process to facilitate the identification of an aptamer that is refractory to the presence of the polyanion. In this context, “polyanionic refractory aptamer” is an aptamer that is capable of forming an aptamer/target complex that is less likely to dissociate in the solution that also contains the polyanionic refractory material than an aptamer/target complex that includes a nonpolyanionic refractory aptamer. In this manner, polyanionic refractory aptamers can be used in the performance of analytical methods to detect the presence or amount or concentration of a target in a sample, where the detection method includes the use of the polyanionic material (e.g. dextran sulfate) to which the aptamer is refractory.

Thus, in some embodiments, a method for producing a polyanionic refractory aptamer is provided. After contacting a candidate mixture of nucleic acids with the target, the target and the nucleic acids in the candidate mixture are allowed to come to equilibrium. A polyanionic competitor is introduced and allowed to incubate in the solution for a period of time sufficient to ensure that most of the fast off rate aptamers in the candidate mixture dissociate from the target molecule. Also, aptamers in the candidate mixture that may dissociate in the presence of the polyanionic competitor will be released from the target molecule. The mixture is partitioned to isolate the high affinity, slow off-rate aptamers that have remained in association with the target molecule and to remove any uncomplexed materials from the solution. The aptamer can then be released from the target molecule and isolated. The isolated aptamer can also be amplified and additional rounds of selection applied to increase the overall performance of the selected aptamers. This process may also be used with a minimal incubation time if the selection of slow off-rate aptamers is not needed for a specific application.

Thus, in some embodiments a modified SELEX process is provided for the identification or production of aptamers having slow (long) off rates wherein the target molecule and candidate mixture are contacted and incubated together for a period of time sufficient for equilibrium binding between the target molecule and nucleic acids contained in the candidate mixture to occur. Following equilibrium binding an excess of competitor molecule, e.g., polyanion competitor, is added to the mixture and the mixture is incubated together with the excess of competitor molecule for a predetermined period of time. A significant proportion of aptamers having off rates that are less than this predetermined incubation period will dissociate from the target during the predetermined incubation period. Re-association of these “fast” off rate aptamers with the target is minimized because of the excess of competitor molecule which can non-specifically bind to the target and occupy target binding sites. A significant proportion of aptamers having longer off rates will remain complexed to the target during the predetermined incubation period. At the end of the incubation period, partitioning nucleic acid-target complexes from the remainder of the mixture allows for the separation of a population of slow off-rate aptamers from those having fast off rates. A dissociation step can be used to dissociate the slow off-rate aptamers from their target and allows for isolation, identification, sequencing, synthesis and amplification of slow off-rate aptamers (either of individual aptamers or of a group of slow off-rate aptamers) that have high affinity and specificity for the target molecule. As with conventional SELEX the aptamer sequences identified from one round of the modified SELEX process can be used in the synthesis of a new candidate mixture such that the steps of contacting, equilibrium binding, addition of competitor molecule, incubation with competitor molecule and partitioning of slow off-rate aptamers can be iterated/repeated as many times as desired.

The combination of allowing equilibrium binding of the candidate mixture with the target prior to addition of competitor, followed by the addition of an excess of competitor and incubation with the competitor for a predetermined period of time allows for the selection of a population of aptamers having off rates that are much greater than those previously achieved.

In order to achieve equilibrium binding, the candidate mixture may be incubated with the target for at least about 5 minutes, or at least about 15 minutes, about 30 minutes, about 45 minutes, about 1 hour, about 2 hours, about 3 hours, about 4 hours, about 5 hours or about 6 hours.

The predetermined incubation period of competitor molecule with the mixture of the candidate mixture and target molecule may be selected as desired, taking account of the factors such as the nature of the target and known off rates (if any) of known aptamers for the target. Predetermined incubation periods may be chosen from: at least about 5 minutes, at least about 10 minutes, at least about 20 minutes, at least about 30 minutes, at least 45 about minutes, at least about 1 hour, at least about 2 hours, at least about 3 hours, at least about 4 hours, at least about 5 hours, at least about 6 hours.

In other embodiments, a dilution is used as an off-rate enhancement process and incubation of the diluted candidate mixture, target molecule/aptamer complex may be undertaken for a predetermined period of time, which may be chosen from: at least about 5 minutes, at least about 10 minutes, at least about 20 minutes, at least about 30 minutes, at least about 45 minutes, at least about 1 hour, at least about 2 hours, at least about 3 hours, at least about 4 hours, at least about 5 hours, at least about 6 hours.

Some embodiments of the present disclosure are concerned with the identification, production, synthesis and use of slow off-rate aptamers. These are aptamers which have a rate of dissociation (ti/2) from a non-covalent aptamer-target complex that is higher than that of aptamers normally obtained by conventional SELEX. For a mixture containing non-covalent complexes of aptamer and target, the ti/2 represents the time taken for half of the aptamers to dissociate from the aptamer-target complexes. The ti/2 of slow dissociation rate aptamers according to the present disclosure is chosen from one of: greater than or equal to about 30 minutes; between about 30 minutes and about 240 minutes; between about 30 minutes to about 60 minutes; between about 60 minutes to about 90 minutes, between about 90 minutes to about 120 minutes; between about 120 minutes to about 150 minutes; between about 150 minutes to about 180 minutes; between about 180 minutes to about 210 minutes; between about 210 minutes to about 240 minutes.

A characterizing feature of an aptamer identified by a SELEX procedure is its high affinity for its target. An aptamer will have a dissociation constant (k_{d}) for its target that is chosen from one of: less than about 1 μM, less than about 100 nM, less than about 10 nM, less than about 1 nM, less than about 100 μM, less than about 10 μM, less than about 1 μM.

### Libraries of Oligonucleotides

In some embodiments, libraries of oligonucleotides comprising random sequences are provided. Such libraries may be useful, in some embodiments, for performing SELEX. In some embodiments, each oligonucleotide of a library of oligonucleotides comprises a number of randomized positions, such as at least 20, 25, 30, 35, 40, 45, or 50, or 20-100, 20-80, 20-70, 20-60, 20-50, 20-40, or 30-40 randomized positions. In some embodiments, each oligonucleotide of a library of oligonucleotides comprises fixed sequences flanking the randomized positions. Such fixed flanking sequences may be the same or different from one another (i.e., the 5′ flanking sequence and the 3′ flanking sequence may be the same or different), and may, in some embodiments, be the same for all members of the library (i.e., all members of the library may have the same 5′ flanking sequence, and/or all members of the library may have the same 3′ flanking sequence).

In some embodiments, the randomized positions may be made up of four or more different nucleotide bases, one or more of which is modified. In some embodiments, all of one type of nucleotide base is modified or unmodified (e.g., all of the cytidines in the randomized region or modified, or all are unmodified). In some embodiments, one type of nucleotide base in the randomized region is present in both modified and unmodified forms. In some such embodiments, the randomized positions are made up of two modified and two unmodified nucleotide bases. In some such embodiments, the randomized positions are made up of adenine, guanine, C5-modified cytidine, and C5-modified uridine. Nonlimiting exemplary C5-modified cytidines and C5-modified uridines are shown in FIGS. 2-6. Libraries of oligonucleotides and methods of making them are further described, e.g., in the Examples herein.

### Exemplary Aptamers

In some embodiments, aptamers that bind a target molecule are provided. In some embodiments, the target molecule is a target protein.

In some embodiments, aptamers that bind from IL-33 are provided.

In some embodiments, an aptamer that binds IL-33 is 15 to 100, or 15 to 90, or 15 to 80, or 15 to 70, or 15 to 60, or 15 to 50, 20 to 100, or 20 to 90, or 20 to 80, or 20 to 70, or 20 to 60, or 20 to 50, or 30 to 100, or 30 to 90, or 30 to 80, or 30 to 70, or 30 to 60, or 30 to 50, or 40 to 100, or 40 to 90, or 40 to 80, or 40 to 70, or 40 to 60, or 40 to 50 nucleotides in length.

In some embodiments, an aptamer that binds XIAP is provided.

In some embodiments, an aptamer that binds XIAP is 15 to 100, or 15 to 90, or 15 to 80, or 15 to 70, or 15 to 60, or 15 to 50, 20 to 100, or 20 to 90, or 20 to 80, or 20 to 70, or 20 to 60, or 20 to 50, or 30 to 100, or 30 to 90, or 30 to 80, or 30 to 70, or 30 to 60, or 30 to 50, or 40 to 100, or 40 to 90, or 40 to 80, or 40 to 70, or 40 to 60, or 40 to 50 nucleotides in length.

In some embodiments, an aptamer that binds K-Ras is provided.

In some embodiments, an aptamer that binds K-Ras is 15 to 100, or 15 to 90, or 15 to 80, or 15 to 70, or 15 to 60, or 15 to 50, 20 to 100, or 20 to 90, or 20 to 80, or 20 to 70, or 20 to 60, or 20 to 50, or 30 to 100, or 30 to 90, or 30 to 80, or 30 to 70, or 30 to 60, or 30 to 50, or 40 to 100, or 40 to 90, or 40 to 80, or 40 to 70, or 40 to 60, or 40 to 50 nucleotides in length.

In some embodiments, an aptamer that binds TNF-alpha is provided.

In some embodiments, an aptamer that binds TNF-alpha is 15 to 100, or 15 to 90, or 15 to 80, or 15 to 70, or 15 to 60, or 15 to 50, 20 to 100, or 20 to 90, or 20 to 80, or 20 to 70, or 20 to 60, or 20 to 50, or 30 to 100, or 30 to 90, or 30 to 80, or 30 to 70, or 30 to 60, or 30 to 50, or 40 to 100, or 40 to 90, or 40 to 80, or 40 to 70, or 40 to 60, or 40 to 50 nucleotides in length.

In some embodiments, the aptamer that binds a target molecule (e.g., any of IL-33, XIAP, K-Ras, TNF-alpha) comprises a region at the 5′ end of the aptamer that is at least 10, at least 15, at least 20, at least 25 or at least 30 nucleotides in length, or 5 to 30, 10 to 30, 15 to 30, 5 to 20, or 10 to 20 nucleotides in length, wherein the region at the 5′ end of the aptamer lacks 5-position modified pyrimidines.

In some embodiments, the aptamer that binds a target molecule (e.g., any of IL-33, XIAP, K-Ras, TNF-alpha) comprises a region at the 3′ end of the aptamer that is at least 10, at least 15, at least 20, at least 25 or at least 30 nucleotides in length, or 5 to 30, 10 to 30, 15 to 30, 5 to 20, or 10 to 20 nucleotides in length, wherein the region at the 3′ end of the aptamer lacks 5-position modified pyrimidines.

In some embodiments, a method of treating or preventing traumatic brain injury (TBI) or rheumatoid arthritis is provided, comprising administering to a subject in need thereof an aptamer provided herein.

### Salts

It may be convenient or desirable to prepare, purify, and/or handle a corresponding salt of the compound, for example, a pharmaceutically-acceptable salt. Examples of pharmaceutically acceptable salts are discussed in Berge et al. (1977) “Pharmaceutically Acceptable Salts” J. Pharm. Sci. 66:1-19.

For example, if the compound is anionic, or has a functional group which may be anionic (e.g., —COOH may be —COO—), then a salt may be formed with a suitable cation.

Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R^{X+}, NH₂R^{X}₂⁺, NHR^{X}₃⁺, NR^{X}₄⁺) Examples of some suitable substituted ammonium ions are those derived from: ethylamine, diethylamine, dicyclohexylamine, triethylamine, butylamine, ethylenediamine, ethanolamine, diethanolamine, piperizine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Kits Comprising Aptamers

The present disclosure provides kits comprising any of the aptamers described herein. Such kits can comprise, for example, at least one aptamer; and components can optionally include at least one of, for example: (a) a pharmaceutically acceptable carrier, such as a solvent or solution; (b) a pharmaceutically acceptable excipient, such as a stabilizer or buffer; (c) at least one container, vial, or apparatus for holding and/or mixing the kit components; and (d) a delivery apparatus. The kit can optionally further comprise one or more of (e) labeling agents useful to detect a target molecule that is bound to an aptamer; (f) a solid support, such as a microarray or bead; and (g) reagents related to quantitation of polymerase chain reaction products, such as intercalating fluorescent dyes or fluorescent DNA probes.

### EXAMPLES

The following examples are presented in order to more fully illustrate some embodiments of the invention. They should, in no way be construed, however, as limiting the broad scope of the invention. Those of ordinary skill in the art can readily adopt the underlying principles of this discovery to design various compounds without departing from the spirit of the current invention.

### General Procedure for Anion Exchange HPLC Purification of Nucleoside Triphosphates.

Nucleoside triphosphates were purified via anion exchange chromatography using an HPLC column packed with Source Q resin (GE Healthcare), installed on a preparative HPLC system, with detection at 278 nm. The linear elution gradient employed two buffers, (buffer A: 10 mM triethylammonium bicarbonate/10% acetonitrile, and buffer B: 1 M triethylammonium bicarbonate/10% acetonitrile), with the gradient running at ambient temperature from low buffer B content to high buffer B over the course of the elution. The desired product was typically the final material to elute from the column and was observed as a broad peak spanning approximately ten to twelve minutes retention time (early eluting products included a variety of reaction by-products, the most significant being the nucleoside diphosphate). Several fractions were collected during product elution. Fractions were analyzed by reversed phase HPLC on a Waters 2795 HPLC with a Waters Symmetry column (PN:WAT054215). Pure product-containing fractions (typically >90%) were evaporated in a Genevac HT-12 evaporator to afford colorless to light tan resins.

### General Procedure for Reversed Phase HPLC Purification of Nucleoside Triphosphates.

Nucleoside triphosphates were purified via reversed phase chromatography using Waters Novapak C8, 30 mm×300 mm column (PN: 186002473), installed on a Waters preparative HPLC system, with detection at 278 nm. The linear elution gradient employed two buffers, (buffer A: 100 mM triethylammonium bicarbonate, and buffer B: 100% acetonitrile), with the gradient running at ambient temperature from low buffer B content to high buffer B over the course of the elution. The desired product was typically the final material to elute from the column and was observed as a broad peak spanning approximately five to twelve minutes retention time (early eluting products included a variety of reaction by-products). Several fractions were collected during product elution. Fractions were analyzed by reversed phase HPLC on a Waters 2795 HPLC with a Waters Symmetry column (PN:WAT054215). Pure product-containing fractions (typically >90%) were evaporated in a Genevac HT-12 evaporator to afford colorless to light tan resins.

Fractions were reconstituted in deionized water and pooled for final analysis. Product quantification was performed by analysis using a Hewlett Packard 8452A Diode Array Spectrophotometer at 278 nm. Product yields were calculated via the equation A=εCL, where A is the UV absorbance, ε is the estimated extinction coefficient and L is the pathlength (1 cm).

### Example 1: Preparation of Modified Deoxycytidines

### Step 1: Synthesis of 5-(N-carboxamide)-2′-deoxycytidine derivatives (Scheme 1, product 2)

Commercially available 5-iodo-2′-deoxycytidine (Scheme 1, product 1) was charged into a round-bottomed flask, dissolved in anhydrous N,N-dimethylformamide (DMF). The starting material was converted to the corresponding N-substituted carboxamide by treatment with the requisite aromatic primary amine (RCH₂NH₂, 4-8 eq.), carbon monoxide (</=1 atm) and (Ph₃P)₄Pd (2 mol %) at room temperature for 24-48 hours. Reaction progress was monitored by thin-layer chromatography (silica gel, eluent: 8-12% methanol/dichloromethane) or reversed phase HPLC (Waters 2795 HPLC with a 2489 detector and using a Waters Symmetry column, buffer A: 100 mM triethylammonium acetate, buffer B: acetonitrile, gradient: 30%-70% buffer B over 30 minutes). The resulting crude reaction mixture was filtered through a Celite bed to remove excess catalyst and solid biproducts. The filtrate was then diluted in dichloromethane and washed with deionized water to remove excess DMF, resulting in a white to off-white crystalline solid forming in the dichloromethane layer. The organic layer containing the solid material was collected into a Schott bottle and stirred at room temperature for several hours to overnight, then filtered, washing the solids with methylene chloride. The filter cake was dried in vacuo and the resulting white to off-white solid 5-modified cytidine carboxamide was recovered in approximately 50%-70% yield.

### Step 2: Synthesis of 4-N-acetyl-5-(N-carboxamide)-2′-deoxycytidine derivatives (Scheme 1, product 3)

The product of Step 1 was charged into a round-bottomed flask, dissolved in anhydrous N,N-dimethylformamide (DMF) and treated with the appropriate anhydride (acetic anhydride or propionic anhydride, 2 equivalents) and the mixture was stirred at room temperature to 40° C. under argon for a minimum of 18 hours. Reaction progress was monitored by HPLC (Waters 2795 HPLC with a 2489 detector and using a Waters Symmetry column, buffer A: 100 mM triethylammonium acetate, buffer B: acetonitrile, gradient: 30%-70% buffer B over 30 minutes). Upon reaction completion, the crude mixture was diluted in ethyl acetate, transferred to a separatory funnel and washed with 2% sodium bicarbonate solution (1×), deionized water (1×) and brine (1×). The organic layer was collected into a Schott bottle and stirred at room temperature for several hours to overnight during which white to off-white crystalline solids formed. The mixture was filtered, and the solids and glassware were washed with ethyl acetate. The filter cake was dried in vacuo and the resulting white to off-white solid 4-N-acetyl-5-modified cytidine carboxamide was recovered in approximately 50%-70% yield.

### Step 3: Synthesis of 5′-O-(4,4′-Dimethoxytrityl)-4-N-acetyl-5-(N-carboxamide)-2′-deoxycytidine derivatives (Scheme 1, product 4)

In a round-bottomed flask with magnetic stirring, the product of Step 2 was dissolved in anhydrous pyridine under argon. Over the course of one hour, 4,4′-dimethoxytrityl chloride (1.1 equivalents) was added in four to five portions to the stirring mixture. The reaction was stirred an additional hour, then quenched with ethanol (6 equivalents) and the reaction mixture was evaporated to a tacky residue. The crude material was dissolved in ethyl acetate and washed with 2% sodium bicarbonate (1×), dried over sodium sulfate, filtered and evaporated to an orangey-yellow foam. The crude material was purified by flash column chromatography (silica gel pretreated with 1% triethylamine/99% ethyl acetate; product eluted with 75% ethyl acetate/25% hexanes). Product-containing fractions were concentrated to provide a white to off-white foam in a 70%-80% yield.

### Step 4: Synthesis of 5′-O-(4,4′-Dimethoxytrityl)-4-N-acetyl-5-(N-carboxamide)-2′-deoxycytidine-3′—O-(N,N-diisopropyl-O-2-cyanoethylphosphoramidite) derivatives (Scheme 1, product 5)

In a round-bottomed flask with magnetic stirring, the product of Step 3 was dissolved in anhydrous dichloromethane under argon. To the reaction mixture was added 2-cyanoethyl-N,N,N′,N′-tetraisopropylphosphine (1.5 equivalents) followed by pyridine trifluoroacetate (1.7 equivalents). The reaction was stirred for 30-60 minutes, then analyzed by thin-layer chromatography (silica gel, eluent: 50-60% ethyl acetate/hexanes), which showed the reaction was complete. The crude mixture was applied to a silica gel flash column preconditioned with 1% triethylamine/99% ethyl acetate and equilibrated with 60% ethyl acetate/40% hexanes. The product was eluted with the same mobile phase, chilled to 0° C. and sparged with argon and collected into argon-purged bottles. Product-containing fractions were concentrated to provide a white to off-white foam in a 70%-80% yield.

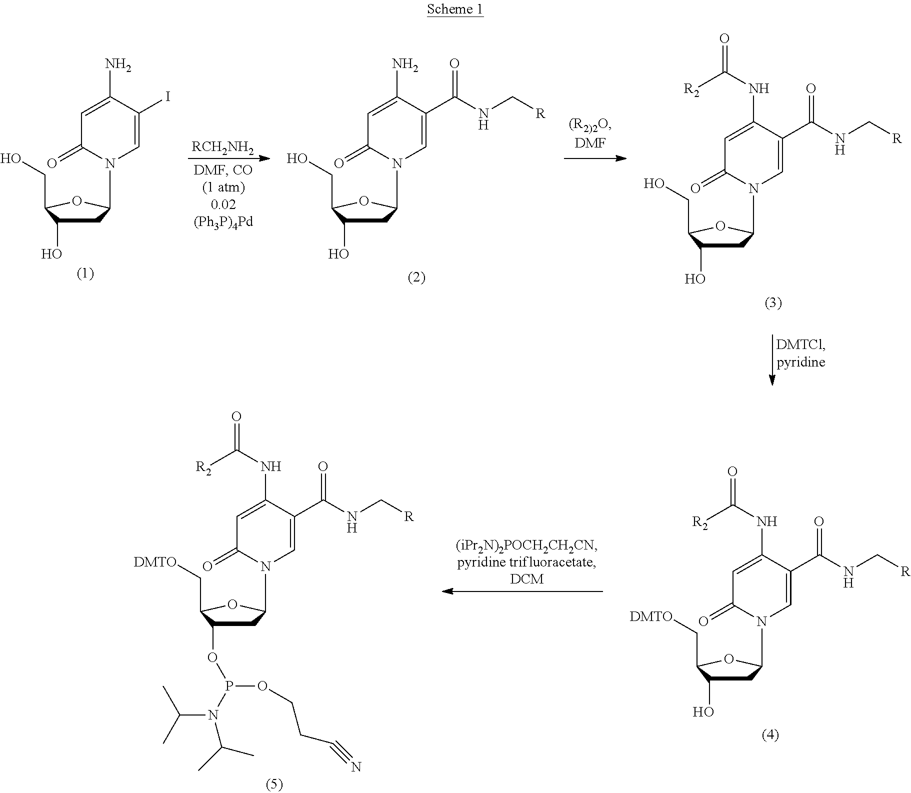

For preparation of 2-cyanoethylphosphoramidite reagents (CEP reagents), the 5-(N-carboxamide)-2′-deoxycytidine derivatives were selectively N-protected, then 5′O-protected as the (4,4′-dimethoxytrityl)-derivatives (4) by reaction with 4,4′-dimethoxytrityl chloride in pyridine (see, e.g. Ross et al., Nucleosides, Nucleotides & Nucleic Acids, 25, 765-770 (2006)). Synthesis of the high purity (>98%) CEP reagents (5) was completed by pyridinium trifluoroacetate catalyzed condensation of the 3′-alcohol with 2-cyanoethyl-N,N,N′,N′-tetraisopropyl phosphine (see, e.g., Sanghvi, et al., Organic Process Research & Development, 4, 175-181 (2000)) and final purification by silica gel flash chromatography with degassed solvents (see, e.g., Still et al., J. Org. Chem., 43, 2923-2925 (1978)).

For preparation of 5′-triphosphate reagents (TPP reagent, Scheme 2), the 5′O-DMT-protected nucleosides (Scheme 1 or 2, product 4) were acetylated with acetic anhydride in pyridine, followed by cleavage of the DMT and 4-N-acetyl protecting groups with 1,1,1,3,3,3,-hexafluoro-2-propanol (Scheme 2, product 6) (see, e.g., Leonard and Neelima, Tetrahedron Letters, 36(43), 7833-7836 (1995)). The resulting crystalline 3′-O-acetated nucleosides were converted into the crude 5′-O-triphosphates by the Ludwig-Eckstein process (Ludwig, J. and Eckstein, F. J. Org. Chem., 1989, 54:631) (Scheme 2, product 7). These chemically modified nucleotides generally require a two-stage purification process: anion-exchange chromatography (AEX) followed by reversed phase preparative HPLC in order to obtain high purity (>90%).

### Example 1-1: Preparation of 5-[(N-(3,3-diphenylpropyl)carboxamide]-2′-deoxycytidine (DPPdC) derivatives

Synthesis of 5-[N-(3,3-diphenylpropyl)carboxamide]-2′-deoxycytidine nucleoside (Scheme 1, product 2): Commercially available 5-iodo-2′-deoxycytidine (Scheme 1, product 1, 20 g, 56.8 mmol) was charged into a round-bottomed flask, dissolved in anhydrous N,N-dimethylformamide (DMF, 137 mL). The starting material was converted to the corresponding N-substituted carboxamide by treatment with the requisite aromatic primary amine (3,3-diphenylpropylamine, (51.6 g, 244 mmol, 4.3 eq.), carbon monoxide (</=1 atm), bis (dibenzylidene acetone) palladium (0) (1.14 g, 2 mmol, 0.035 eq) and triphenylphosophene (2.34 g, 8.5 mmol, 0.15 eq) at room temperature for 24-48 hours. Reaction progress was monitored by thin-layer chromatography (silica gel, eluent: 8-12% methanol/dichloromethane) or reversed phase HPLC (Waters 2795 HPLC with a 2489 detector and using a Waters Symmetry column, buffer A: 100 mM triethylammonium acetate, buffer B: acetonitrile, gradient: 30%-70% buffer B over 30 minutes). The resulting crude reaction mixture was filtered through a Celite bed to remove excess catalyst and solid byproducts. The filtrate was then diluted in dichloromethane and washed with deionized water to remove excess DMF, resulting in a white to off-white crystalline solid forming in the dichloromethane layer. The organic layer containing the solid material was collected into a Schott bottle and stirred at room temperature for several hours to overnight, then filtered, washing the solids with methylene chloride. The filter cake was dried in vacuo and the resulting white to off-white solid 5-modified cytidine carboxamide was recovered (14.65 g, 56% yield). ¹H-NMR (300 mHz, DMSO-d₆): δ=8.37 (s, 1H), 8.14 (t, J=5.3 Hz, 1H), 7.99 (bs, 1H), 7.69 (bs, 1H), 7.24-7.33 (m, 8H), 7.13-7.21 (m, 2H), 6.12 (t, J=6.3 Hz, 1H), 5.24 (d, J=4.2 Hz, 1H), 5.09 (t, J=5.6 Hz), 4.22-4.30 (m, 1H), 4.03 (t, J=7.8 Hz, 1H), 3.83 (t, J_{A}=7.5, J_{B}=3.9 Hz, 1H), 3.55-3.72 (m, 2H), 3.08 (dd, J_{A}=13.5, J_{B}=6.0 Hz, 2H), 2.10-2.32 (m, 4H). ¹³C-NMR (100 mHz, DMSO-d₆): δ=170.53 (1C), 165.72 (1C), 163.98 (1C), 153.93 (1C), 145.19 (2C), 145.14 (2C), 144.13 (1C), 128.91 (4C), 128.09 (2C), 128.07 (2C), 126.59 (2C), 99.74 (1C), 86.44 (1C), 85.59 (1C), 75.07 (1C), 61.68 (1C), 48.52 (1C), 38.37 (1C), 37.93 (1C), 34.67 (1C), 21.32 (1C). MS (m/z) calcd for C₂₅H₂₈N₄O₅, 464.52; found 463.2 [M-H]⁻ (ESI⁻).

Synthesis of 4-N-acetyl-5-[3,3-diphenylpropyl)carboxamide]-2′-deoxycytidine nucleoside (Scheme 1, 1, product 3): The product of the previous step (Scheme 1, product 2, 14.5 g, 31.2 mmol)) was charged into a round-bottomed flask, dissolved in anhydrous N,N-dimethylformamide (DMF, 284 mL) and treated with acetic anhydride (5.9 mL, 62 mmol, 2 eq) and the mixture was stirred at room temperature to 40° C. under argon for approximately 5.5 hours. Reaction progress was monitored by HPLC (Waters 2795 HPLC with a 2489 detector and using a Waters Symmetry column, buffer A: 100 mM triethylammonium acetate, buffer B: acetonitrile, gradient: 30%-70% buffer B over 30 minutes). Upon reaction completion, the crude mixture was diluted in ethyl acetate, transferred to a separatory funnel and washed with 2% sodium bicarbonate solution (1×), deionized water (1×) and brine (1×). The organic layer was collected into a Schott bottle and stirred at room temperature for several hours to overnight during which fine crystalline solids formed. The mixture was filtered, and the solids and glassware were washed with ethyl acetate. The filter cake was dried in vacuo and the resulting white to off-white solid product was recovered (12.04 g, 76% yield). ¹H-NMR (300 mHz, DMSO-d₆): δ=11.32 (s, 1H), 8.70 (s, 1H), 8.36-8.45 (m, 1H), 7.23-7.36 (m, 8H), 7.11-7.20 (m, 2H), 6.09 (t, J=5.7 Hz, 1H), 5.24-5.31 (m, 1H), 4.22-4.32 (m, 1H), 3.88-3.94 (m, 1H), 3.57-3.75 (m, 2H), 3.05-3.16 (m, 2H), 2.36-2.41 (bs, 3H), 2.14-2.36 (m, 4H). ¹³C-NMR (100 mHz, DMSO-d₆): δ=170.63 (1C), 164.80 (1C), 159.17 (1C), 152.64 (1C), 145.45 (1C), 144.58/144.56 (2C), 144.56 (1C), 128.36 (4C), 127.52 (4C), 126.03 (2C), 99.94(1C), 88.09 (1C), 86.89 (1C), 69.35 (1C), 60.55 (1C), 47.92 (1C), 40.51 (1C), 37.98 (1C), 33.91 (1C), 26.10 (1C). MS (m/z) calcd for C₂₇H₃₀N₄O₆, 506.56; found 505.3 [M-H]⁻ (ESI⁻).

Synthesis of 5′-O-(4,4′-Dimethoxytrityl)-4-N-acetyl-5-[N-(3,3-diphenylpropyl)carboxamide]-2′-deoxycytidine (Scheme 1, product 4): In a round-bottomed flask with magnetic stirring, the product of the previous step (Scheme 1, product 3, 12.2 g, 24.1 mmol) was dissolved in anhydrous pyridine (80 mL), under argon. Over the course of one hour, 4,4′-dimethoxytrityl chloride (9.2 g, 27.0 mmol, 1.1 eq) was added in four portions to the stirring mixture. The reaction was stirred an additional hour, then quenched with ethanol (9.5 mL, 163 mmol, 6.8 equivalents) and the reaction mixture was evaporated to a tacky residue.

The crude material was dissolved in ethyl acetate, transferred to a separatory funnel and washed with 2% sodium bicarbonate (1×). The organic layer was collected and dried over sodium sulfate, filtered and evaporated to an orange-yellow foam. The crude material was purified by flash column chromatography (silica gel pretreated with 1% triethylamine/99% ethyl acetate; product eluted with 80%-90% ethyl acetate/20%-10% hexanes). Product-containing fractions were concentrated to provide a white to off-white foam (15.27 g, 78% yield). ¹H-NMR (300 mHz, DMSO-d₆): δ=11.36 (s, 1H), 8.57 (t, J=4.5 Hz, 1H), 8.37 (s, 1H), 7.09-7.39 (m, 19H), 6.72-6.87 (d, 4H), 6.10 (t, J=6.0 Hz, 1H), 5.15 (d, J=4.5 Hz, 1H), 4.35 (dt, J_{A}=9.9, J_{B}=4.5 Hz 1H), 4.08-3.98 (m, 1H), 3.92 (t, J=7.5 Hz, 1H), 3.68 (d, J=2.4 Hz, 6H), 3.17-3.31 (m, 2H), 2.78-3.02 (m, 2H), 2.33-2.46 (m, 4H), 2.17-2.29 (m, 1H), 1.92-2.11 (m, 2H). ¹³C-NMR (100 mHz, DMSO-d₆): δ=170.61 (1C), 164.61 (1C), 159.26 (1C), 157.93/157.90 (2C), 152.54 (1C), 144.97 (1C), 144.65 (1C), 144.23 (2C), 135.40/135.35 (2C), 129.57 (1C), 129.49 (1C), 128.34 (4C), 127.66 1C), 127.54 (1C), 127.41/127.40 (4C), 126.51 (1C), 126.04 (2C), 113.00 (4C), 99.94(1C), 87.39 (1C), 86.32 (1C), 85.60 (1C), 69.90 (1C), 59.66 (1C), 54.85/54.82 (2C), 48.27 (1C), 40.37 (1C), 38.16 (1C), 33.73 (1C), 26.13 (1C). MS (m/z) calcd for C₄₈H₄₈N₄O₈, 808.93; found 807.3 [M-H]⁻ (ESI⁻).

Synthesis of 5′-O-(4,4′-Dimethoxytrityl)-4-N-acetyl-5-[N-(3,3-diphenylpropyl)carboxamide]-2′-deoxycytidine-3′-O-(N,N-diisopropyl-O-2-cyanoethylphosphoramidite) (Scheme 1, product 5): In a round-bottomed flask with magnetic stirring, the product of the previous step (Scheme 1, product 4, 14.0 g, 17.3 mmol) was dissolved in anhydrous dichloromethane (43 mL) under argon. To the reaction mixture was added 2-cyanoethyl-N,N,N′,N′-tetraisopropylphosphine (8.2 mL, 26 mmol, 1.5 eq) followed by pyridinium trifluoroacetate (5.4 g, 28.2 mmol, 1.6 eq). The reaction was stirred for 30 minutes, then analyzed by thin-layer chromatography (silica gel, eluent: 60% ethyl acetate/40% hexanes), which showed the reaction was complete. The crude mixture was applied to a silica gel flash column preconditioned with 1% triethylamine/99% ethyl acetate and equilibrated with 60% ethyl acetate/40% hexanes. The product was eluted with the same mobile phase, chilled to 0° C. and sparged with argon and collected into argon-purged bottles. Product-containing fractions were concentrated to provide a white to off-white foam (13.85 g, 79% yield). ¹H-NMR (300 mHz, DMSO-d₆): δ=11.36 (s, 1H), 8.56 (bt, 1H), 8.41-8.47 (d, 1H), 7.11-7.37 (m, 19H), 6.74-6.83 (d, 4H), 6.03-6.10 (m, 1H), 4.30-4.44 (m, 1H), 4.12-4.22 (m, 1H), 3.91 (dd, J_{A}=14.1, J_{B}=7.5 Hz, 1H), 3.63-3.78 (m, 7H), 3.43-3.62 (m, 3H), 3.22-3.37 (m, 2H), 2.79-3.02 (m, 2H), 2.75 (t, J=6.0 Hz, 1H), 2.64 (t, J=6.0, Hz, 1H), 2.33-2.45 (m, 5H), 1.94-2.08 (m, 2H), 1.11 (dd, J_{A}=12.3, J_{B}=6.6 Hz 12H), 0.97 (d, J=6.6 Hz, 3H). ³¹P-NMR (300 mHz, DMSO-d₆): δ=147.56/147.56 (s, 1P). MS (m/z) calcd for C₅₇H₆₅N₆O₉P, 1009.15; found 1007.3 [M-H]⁻ (ESI⁻).

Synthesis of 5-[N-(3,3-diphenylpropyl)carboxamide]-3′-O-acetyl-2′-deoxycytidine nucleoside (Scheme 2, product 6): In a round-bottomed flask with magnetic stirring, the starting material (Scheme 2, product 4, 1.26 g, 1.56 mmol) was dissolved in anhydrous pyridine (10 mL) under argon. Acetic anhydride (1 mL, 10.5 mmol, 6.7 eq) dropwise to the stirring mixture. The reaction was stirred 29 hours and reaction progress was monitored by HPLC (Waters 2795 HPLC with a 2489 detector and using a Waters Symmetry column, buffer A: 100 mM triethylammonium acetate, buffer B: acetonitrile, gradient: 75% buffer B, isocratic over 30 minutes). The crude mixture was evaporated, with two acetone co-evaporations to recover a pale yellow foam. The residue was dissolved in 1,1,1,3,3,3-hexafluoro-2-propanol (10 mL, 95 mmol)) (Leonard, N. J. Tetrahedron Letters, 1995, 36:7833) and heated at approximately 50° C. for 16 hours. Complete cleavage of the DMT group was confirmed by TLC (5% methanol/dichloromethane). The red solution was quenched by pouring into well-stirred methanol (approximately 75 mL). The resulting yellow solution was concentrated in vacuo and the residue was dissolved in hot ethyl acetate (20 mL). The product crystallized upon cooling and the resulting slurry was stirred at 0° C., followed by filtration and washing with ethyl acetate. The 3′-O-acetyl-nucleoside (product 6) was isolated as a white solid (0.55 g, 70% yield). ¹H-NMR (300 mHz, DMSO-d₆): δ=8.38 (s, 1H), 8.26 (bt, J=4.8 Hz, 1H), 8.04 (bs, 1H), 7.77 (bs, 1H), 7.24-7.41 (m, 8H), 7.12-7.23 (m, 2H), 6.17 (t, J=6.9 Hz, 1H), 5.23-5.30 (m, 1H), 5.18 (t, J=5.3 Hz, 1H), 3.98-4.14 (m, 2H), 3.62-3.77 (m, 2H), 3.10 (dd, J_{A}=12.3, J_{B}=6.6 Hz, 2H), 2.35-2.48 (m, 2H), 2.22-2.35 (m, 2H), 2.08 (s, 3H). ¹³C-NMR (100 mHz, DMSO-d₆): δ=170.53 (1C), 165.72 (1C), 163.98 (1C), 153.93 (1C), 145.19 (2C), 145.14 (2C), 144.13 (1C), 128.91 (4C), 128.09 (2C), 128.07 (2C), 126.59 (2C), 99.74 (1C), 86.44 (1C), 85.59 (1C), 75.07 (1C), 61.68 (1C), 48.52 (1C), 38.37 (1C), 37.93 (1C), 34.67 (1C), 21.32 (1C). MS (m/z) calcd for C₂₇H₃₀N₄O₆, 506.56; found 506.2 [M-H]⁻ (ESI⁻).

Synthesis of 5-[N-(3,3-diphenylpropyl)carboxamide]-2′-deoxycytidine-5′-O-triphosphate (tris-triethylammonium salt) (7): The triphosphate (7) was synthesized from the 3′-O-acetyl-nucleoside (6) by the procedure of Ludwig and Eckstein (Ludwig, J. and Eckstein, F. J. Org. Chem. 1989, 54:631) at 500 μmol-scale (5×). The crude triphosphate product, after ammonolysis and evaporation, was purified by anion exchange chromatography and reversed phase chromatography, as described in the General Procedures (above). [F est. 13.700 cm⁻¹ M⁻¹] the isolated purified product was 59.8 μmol (12% yield). ¹H-NMR (300 mHz, D₂O): δ=7.95 (s, 1H), 7.22-7.28 (m, 4H), 7.11-7.19 (m, 4H), 6.97-7.05 (m, 2H), 6.05 (t, J=6.8 Hz, 1H), 4.51 (quintet, J=3.0 Hz, 1H), 4.10-4.17 (m, 3H), 3.92 (t, J=7.5 Hz, 1H), 3.29-3.40 (m, 1H), 3.18-3.29 (m, 1H), 3.00 (q, J=7.5 Hz, 19H), 2.25-2.37 (m, 3H), 2.11-2.22 (m, 1H), 1.11 (t, J=7.2 Hz, 29H). ¹³C-NMR (80 mHz, DMSO-d₆): δ=165.52 (1C), 163.55 (1C), 155.78 (1C), 145.35/145.38 (2C), 142.70 (1C), 128.81 (4C), 127.45 (4C), 126.18/126.20 (2C), 100.74 (1C), 86.84 (1C), 85.86/85.98 (1C), 70.70 (1C), 65.27/65.34 (1C), 49.60 (1C), 46.50 (3C), 39.76 (1C), 39.01 (1C), 33.50 (1C), 8.17 (3C). ³¹P-NMR (100 mHz, D₂O): δ=−9.94 (d, J=16.7 Hz, 1P), −11.60 (d, J=17.0 Hz, 1P), −23.30 (t, J=17 Hz, 1P). MS (m/z) calcd for C₂₅H₃₀N₄O₁₄P₃, 703.45; found 703.1 [M-H]⁻ (ESI⁻).

### Example 1-2: Preparation of 5-[N-(4-phenylbenzyl)carboxamide]-2′-deoxycytidine (PBndC) derivatives

Synthesis of 5-[N-(4-phenylbenzyl)carboxamide]-2′-deoxycytidine nucleoside (Scheme 1, product 2): Commercially available 5-iodo-2′-deoxycytidine (Scheme 1, product 1, 20.87 g, 69.1 mmol) was charged into a round-bottomed flask, dissolved in anhydrous N,N-dimethylformamide (DMF, 150 mL). The starting material was converted to the corresponding N-substituted carboxamide by treatment with 4-phenylbenzylamine, (47.83 g, 261 mmol, 4.2 eq.), carbon monoxide (</=1 atm), bis(dibenzylidene acetone) palladium (0) (1.25 g, 2.17 mmol, 0.035 eq) and triphenylphosphene (2.54 g, 9.7 mmol, 0.15 eq) at room temperature for 24-48 hours. Reaction progress was monitored by thin-layer chromatography (silica gel, eluent: 8-12% methanol/dichloromethane) or reversed phase HPLC (Waters 2795 HPLC with a 2489 detector and using a Waters Symmetry column, buffer A: 100 mM triethylammonium acetate, buffer B: acetonitrile, gradient: 30%-70% buffer B over 30 minutes). The resulting crude reaction mixture was filtered through a Celite bed to remove excess catalyst and solid biproducts. The filtrate was then diluted in dichloromethane and washed with deionized water to remove excess DMF, resulting in a white to off-white crystalline solid forming in both the aqueous and dichloromethane layer. Each layer containing the solid material was collected separately in into its own Schott bottle and stirred at room temperature for a several hours to overnight, then filtered, washing the solids with methylene chloride. The filter cake was dried in vacuo and the resulting white to off-white solid 5-modified cytidine carboxamide was recovered (17.00 g, 63% yield). ¹H-NMR (300 mHz, DMSO-d₆): δ=8.85 (t, J=5.6 Hz, 1H), 8.49 (s, 1H), 7.62-7.73 (m, 4H), 7.36-7.52 (m, 5H), 6.20 (t, J=6.4 Hz, 1H), 5.30 (d, J=4.4 Hz, 1H), 5.12 (t, J=5.4, 1H), 4.43-4.56 (m, 2H), 4.28-4.34 (1H), 3.88 (dd, J_{A}=7.8, J_{B}=4.2 Hz), 3.60-3.73 (d, J=4.2 Hz, 2H), 2.24 (t, J=6.0, 1H). ¹³C-NMR (100 mHz, DMSO-d₆): δ=166.70 (1C), 164.75 (1C), 154.77 (1C), 145.08 (1C), 141.20 (1C), 140.04 (1C), 139.70 (1C), 130.18 (2C), 129.03 (2C), 128.59 (2C), 127.92 (2C), 127.82 (2C), 99.98 (1C), 88.88 (1C), 87.07 (1C), 71.24 (1C), 62.28 (1C), 43.24 (1C), 41.41 (1C). MS (m/z) calcd for C₂₃H₂₄N₄O₅, 436.47; found 435.2 [M-H]⁻ (ESI⁻).

Synthesis of 4-N-propionyl-5-[(4-phenylbenzyl)carboxamide]-2′-deoxycytidine nucleoside (Scheme 1, product 3): The product of the previous step (Scheme 1, product 2, 16.83 g, 38.62 mmol) was charged into a round-bottomed flask, dissolved in anhydrous N,N-dimethylformamide (DMF, 350 mL) and treated with propionic anhydride (10 mL, 80 mmol, 2 eq) and the mixture was stirred at 40° C. under argon for approximately 6 hours. Reaction progress was monitored by HPLC (Waters 2795 HPLC with a 2489 detector and using a Waters Symmetry column, buffer A: 100 mM triethylammonium acetate, buffer B: acetonitrile, gradient: 30%-70% buffer B over 30 minutes). Upon reaction completion, the crude mixture was diluted in dichloromethane, transferred to a separatory funnel and washed with deionized water (1×). The organic and aqueous layers were collected into separate Schott bottles and stirred at room temperature for a several hours to overnight during which fine crystalline solids formed. The resulting mixtures were filtered, and the solids and glassware were washed with ethyl acetate. The filter cake was dried in vacuo and the resulting white to off-white solid product was recovered (12.74 g, 767% yield). ¹H-NMR (300 mHz, DMSO-d₆): δ=11.39 (s, 1H), 9.03 (t, J=5.7 Hz, 1H), 8.76 (s, 1H), 7.62-7.69 (m, 4H), 7.41-7.50 (m, 4H), 7.33-7.40 (m, 1H), 6.12 (t, J=6.2 Hz, 1H), 5.30 (d, J=4.2 Hz, 1H), 5.13 (t, J=5.3, 1H), 4.40-4.58 (m, 2H), 4.23-4.34 (1H), 3.93 (dd, J_{A}=7.8, J_{B}=3.9 Hz, 1H), 3.58-3.76 (d, J=4.2 Hz, 2H), 2.81 (dd, J_{A}=14.7, J_{B}=7.2 Hz, 2H), 2.31-2.48 (m, 1H), 2.18-2.29 (m, 1H), 1.06 (t, J=7.4 Hz, 3H). ¹³C-NMR (80 mHz, DMSO-d₆): δ=174.58 (1C), 165.58 (1C), 159.75 (1C), 153.28 (1C), 146.18 (1C), 140.42 (1C), 139.38 (1C), 138.46 (1C), 129.39 (2C), 128.41 (2C), 127.83 (1C), 127.14 (2C), 127.06 (2C), 100.49 (1C), 88.69 (1C), 87.49 (1C), 70.15 (1C), 61.22 (1C), 42.75 (1C), 40.98 (1C), 31.74 (1C), 9.04 (1C). MS (m/z) calcd for C₂₆H₂₈N₄O₆, 492.53; found 491.2 [M-H]⁻ (ESI⁻).

Synthesis of 5′-O-(4,4′-Dimethoxytrityl)-4-N-acetyl-5-[N-(4-phenylbenzyl)carboxamide]-2′-deoxycytidine derivatives (Scheme 1, product 4): In a round-bottomed flask with magnetic stirring, the product of the previous step (16.01 g, 32.5 mmol) was dissolved in anhydrous pyridine (108 mL) under argon. Over the course of one hour, 4,4′-dimethoxytrityl chloride (13.13 g, 38.8 mmol, 1.1 eq) was added in five portions to the stirring mixture. The reaction was stirred an additional quarter hour, then quenched with ethanol (11.5 mL, 195 mmol, 6 eq) and the reaction mixture was evaporated to a tacky residue. The crude material was dissolved in warm dichloromethane, transferred to a separatory funnel and washed with 2% sodium bicarbonate (1×). The organic layer was collected and dried over sodium sulfate, filtered and evaporated to an orangey-yellow foam. The crude material was purified by flash column chromatography (silica gel pretreated with 1% triethylamine/99% ethyl acetate; product eluted with 80%-90% ethyl acetate/20%-10% hexanes). Product-containing fractions were concentrated to provide a white to off-white foam (15.27 g, 78% yield). ¹H-NMR (300 mHz, DMSO-d₆): δ=11.49 (s, 1H), 9.13 (t, J=5.3 Hz, 1H), 8.49 (s, 1H), 7.59-7.68 (m, 2H), 7.52-7.59 (m, 2H), 7.42-7.51 (m, 2H), 6.12 (t, J=6.0 Hz, 1H), 5.33 (d, J=4.5 Hz, 1H), 4.26 (d, J=5.1 Hz, 2H), 4.13-4.22 (m, 1H), 4.00-4.09 (m, 1H), 3.71 (bs, 6H), 3.19-3.33 (m, 2H), 2.83 (dd, J_{A}=14.4, J_{B}=7.2, 2H), 2.35-2.46 (m, 1H), 2.21-2.33 (m, 1H), 1.07 (t, J=7.4 Hz, 3H). ¹³C-NMR (100 mHz, DMSO-d₆): δ=174.59 (1C), 165.47 (1C), 159.84 (1C), 158.53 (1C), 153.15 (1C), 146.06 (1C), 145.28 (1C), 140.35 (1C), 139.41 (1C), 138.13 (1C), 136.00 (1C), 130.17 (1C), 130.11 (1C), 129.39 (2C), 128.49 (2C), 128.13 (1C), 127.85 (1C), 127.14 (1C), 127.07 (2C), 127.04 (2C), 113.61 (4C), 100.48 (1C), 87.98 (1C), 86.19 (1C), 70.70 (1C), 64.41 (1C), 55.41 (2C), 42.79 (1C), 40.73 (1C), 31.83 (1C), 9.04 (1C). MS (m/z) calcd for C₄₇H₄₆N₄O₈, 794.90; found 793.3 [M-H]⁻ (ESI⁻).

Synthesis of 5′-O-(4,4′-Dimethoxytrityl)-4-N-acetyl-5-[N-(4-phenylbenzyl)carboxamide]-2′-deoxycytidine-3′-O-(N,N-diisopropyl-O-2-cyanoethylphosphoramidite) (Scheme 1, product 5): In a round-bottomed flask with magnetic stirring, the product of Step 3 (Scheme 1, product 4, 11.88 g, 14.9 mmol) was dissolved in anhydrous dichloromethane (38 mL) under argon. To the reaction mixture was added 2-cyanoethyl-N,N,N′,N′-tetraisopropylphosphine (8 mL, 25.2 mmol, 1.5 eq) followed by pyridine trifluoroacetate (4.75 g, 24.6 mmol, 2.1 eq). The reaction was stirred for 30 minutes, then analyzed by thin-layer chromatography (silica gel, eluent: 60% ethyl acetate/40% hexanes), which showed the reaction was complete. The crude mixture was applied to a silica gel flash column preconditioned with 1% triethylamine/99% ethyl acetate and equilibrated with 60% ethyl acetate/40% hexanes. The product was eluted with the same mobile phase, chilled to 0° C. and sparged with argon and collected into argon-purged bottles. Product-containing fractions were concentrated to provide a white to off-white foam (12.04 g, 81% yield). ¹H-NMR (300 mHz, DMSO-d₆): δ=11.48 (s, 1H), 9.12 (t, J=4.7 Hz, 1H), 8.54/8.57 (s, 1H), 7.59-7.66 (m, 2H), 7.51-7.58 (m, 2H), 7.43-7.50 (m, 2H), 7.18-7.40 (m, 12H). 6.77-6.89 (m, 4H), 6.03-6.13 (m, 1H), 4.12-4.47 (m, 4H), 3.70 (s, 6H), 3.43-3.780 (m, 4H), 3.22-3.36 (m, 2H), 2.82 (dd, J_{A}=14.9, J_{B}=7.4 Hz, 2H), 2.76 (t, J=5.9 Hz, 1H), 2.64 (t, J=5.9 Hz, 1H), 2.36-2.46 (m, 2H), 1.02-1.17 (m, 12H), 0.98 (d, J=6.9 Hz, 2H). ³¹P-NMR (100 mHz, DMSO-d₆): δ=147.55/147.35 (s, 1P). MS (m/z) calcd for C₅₆H₆₃N₆O₉P, 995.13; found 993.4 [M-H]⁻ (ESI⁻).

Synthesis of 5-[N-(4-phenylbenzyl) carboxamide]-3′-O-acetyl-2′-deoxycytidine nucleoside (Scheme 2, product 6): In a round-bottomed flask with magnetic stirring, the starting material (Scheme 2, product 4, 1.26 g, 1.56 mmol) was dissolved in anhydrous pyridine (10 mL) under argon. Acetic anhydride (1 mL, 10.5 mmol, 6.7 eq) dropwise to the stirring mixture. The reaction was stirred 16.5 hours and reaction progress was monitored by TLC (8% methanol/dichloromethane). The crude mixture was evaporated to recover a pale yellow foam. The residue was dissolved in 1,1,1,3,3,3-hexafluoro-2-propanol (10 mL, 95 mmol)) (Leonard, N. J. Tetrahedron Letters, 1995, 36:7833) and heated at approximately 50° C. for 22.25 hours. Complete cleavage of the DMT group was confirmed by TLC (8% methanol/dichloromethane). The red solution was quenched by pouring into well-stirred methanol (approximately 75 mL). The resulting yellow solution was concentrated in vacuo and the residue was dissolved in hot ethyl acetate (20 mL). The product crystallized upon cooling and the resulting slurry was stirred at 0° C., followed by filtration and washing with ethyl acetate. The 3′-O-acetyl-nucleoside (6) was isolated as a white solid (0.45 g, 56% yield). ¹H-NMR (300 mHz, DMSO-d₆): δ=8.86 (t, J=5.7 Hz, 1H), 8.44 (s, 1H), 7.99-8.18 (bs, 1H), 7.75-7.88 (bs, 1H), 7.60-7.68 (m, 4H), 7.32-7.51 (m, 5H), 6.16 (dd, J_{A}=8.1, J_{B}=6.0 Hz, 1H), 5.20-5.27 (m, 1H), 5.14 (t, J=5.7 Hz, 1H), 4.38-4.53 (m, 2H), 4.06 (dd, J_{A}=5.7, J_{B}=3.9 Hz, 1H), 3.59-3.72 (m, 2H), 2.28-2.47 (m, 2H), 2.07 (s, 3H). ¹³C-NMR (80 mHz, DMSO-d₆): δ=170.52 (1C), 165.85 (1C), 164.00 (1C), 153.88 (1C), 144.44 (1C), 140.43 (1C), 139.29 (1C), 138.56 (1C), 129.40 (2C), 128.28 (2C), 127.82 (1C), 127.154 (2C), 127.05 (2C), 99.52 (1C), 86.44 (1C), 85.55 (1C), 75.11 (1C), 61.67 (1C), 42.50 (1C), 37.79 (1C), 21.33 (1C). MS (m/z) calcd for C₂₅H₂₆N₄O₆, 478.51; found 477.2 [M-H]⁻ (ESI⁻).

Synthesis of 5-[N-(4-phenylbenzyl)carboxamide]-2′-deoxyuridine-5′-O-triphosphate (tris-triethylammonium salt) (Scheme 2, product 7): The triphosphate (7) was synthesized from the 3′-O-acetyl-nucleoside (6) by the procedure of Ludwig and Eckstein (Ludwig, J. and Eckstein, F. J. Org. Chem. 1989, 54:631) at 500 μmol-scale (5×). The crude triphosphate product, after ammonolysis and evaporation, was purified by anion exchange chromatography and reversed phase chromatography, as described in the General Procedures (above). [ε_{est.} 13,700 cm⁻¹ M⁻¹] the isolated purified product was 275 μmol (55% yield). ¹H-NMR (300 mHz, D₂O): δ=8.40 (s, 1H), 7.51-7.60 (m, 4H), 7.32-7.43 (m, 4H), 7.25-7.33 (m, 1H), 6.09 (t, J=6.5 Hz, 1H), 4.57 (quintet, J=3.6 mHz, 1H), 4.43 (q, J=15.3 Hz, 2H), 4.11-4.23 (m, 3H), 3.00 (q, J=7.5 Hz, 19H), 2.31-2.42 (m, 1H), 2.16-2.90 (m, 1H), 1.135 (t, J=7.2 Hz, 31H). ¹³C-NMR (75 Hz, D₂O): δ=166.29 (1C), 163.67 (1C), 155.92 (1C), 143.40 (1C), 140.04 (1C), 139.14 (1C), 138.02 (1C), 129.07 (2C), 128.00 (2C), 86.89 (1C), 86.07/85.96 (1C), 70.50 (1C), 65.23/65.16 (1C), 41.01 (3C), 42.95 (1C), 39.88 (1C), 8.16 (3C). ³¹P-NMR (100 mHz, D₂O): δ=8.60 (d, J=16.9 Hz, 1P), −11.40 (d, J=16.8 Hz, 1P), −23.00 (t, J=17.0 Hz, 1P). MS (m/z) calcd for C₂₅H₂₆N₄O₁₄P₃, 675.40; found 675.1 [M-H]⁻ (ESI⁻).

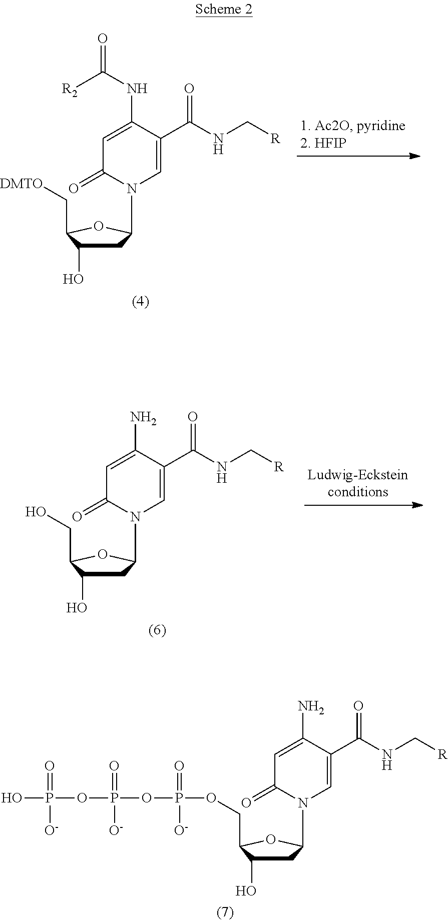

### Example 2: Preparation of Modified Deoxyuridines

### Step 1: Synthesis of 5′-O-(4,4′-Dimethoxytrityl)-5-(N-carboxamide)-2′-deoxyuridine derivatives (Scheme 3, product 9)

The starting material, 5′-O-dimethoxytrityl-5-trifluoroethoxycarbonyl-2′-deoxyuridine (Scheme 3, product 8) was prepared by the procedure of Matsuda et al (Noruma, Y.; Ueno, Y.; Matsuda, A. Nucleic Acids Research 1997, 25:2784-2791; Ito, T.; Ueno, Y.; Matsuda, A. Nucleic Acid Research 2003, 31:2514-2523). A solution of (8), the requisite aromatic primary amine (RCH₂NH₂ (1.3 equivalents), triethylamine (2 equivalents) and anhydrous acetonitrile was heated under an inert atmosphere at 40-50° C. for 18-24 hours. Quantitative conversion of (8) to amide (9) was confirmed by thin layer chromatography (silica gel, 5% methanol/dichloromethane) or HPLC. The reaction mixture was concentrated in vacuo and the residue purified by silica gel flash chromatography (Still, W. C.; Kahn, M.; Mitra, A. J. Org. Chem. 1978, 43:2923) using an eluent of 0-3% methanol in 1% triethylamine/ethyl acetate. Fractions containing pure product were combined and evaporated to afford (9) as a white foam in an 80-90% yield.

### Step 2: Synthesis of 5′-O-(4,4′-Dimethoxytrityl)-5-(N-carboxamide)-2′-deoxyuridine-3′-CE phosphoramidite derivatives (Scheme 3, product 10)

In a round-bottomed flask with magnetic stirring, a DMT-protected nucleoside (9) was dissolved in anhydrous dichloromethane under argon. To the reaction mixture was added 2-cyanoethyl-N,N,N′,N′-tetraisopropyl phosphine (1.05 equivalents) followed by pyridinium trifluoroacetate (1.1 equivalents). The reaction was stirred for 30-60 minutes, then analyzed by thin-layer chromatography (silica gel, eluent: 5% methanol/95% dichloromethane), which showed the reaction was complete. The crude mixture was applied to a silica gel flash column equilibrated with 1% triethylamine/20% hexanes/79% ethyl acetate and the product was eluted with the same mobile phase, chilled to 0° C. and sparged with argon and collected into argon-purged bottles. Product-containing fractions were concentrated to provide a white to off-white foam in an 80%-90% yield.

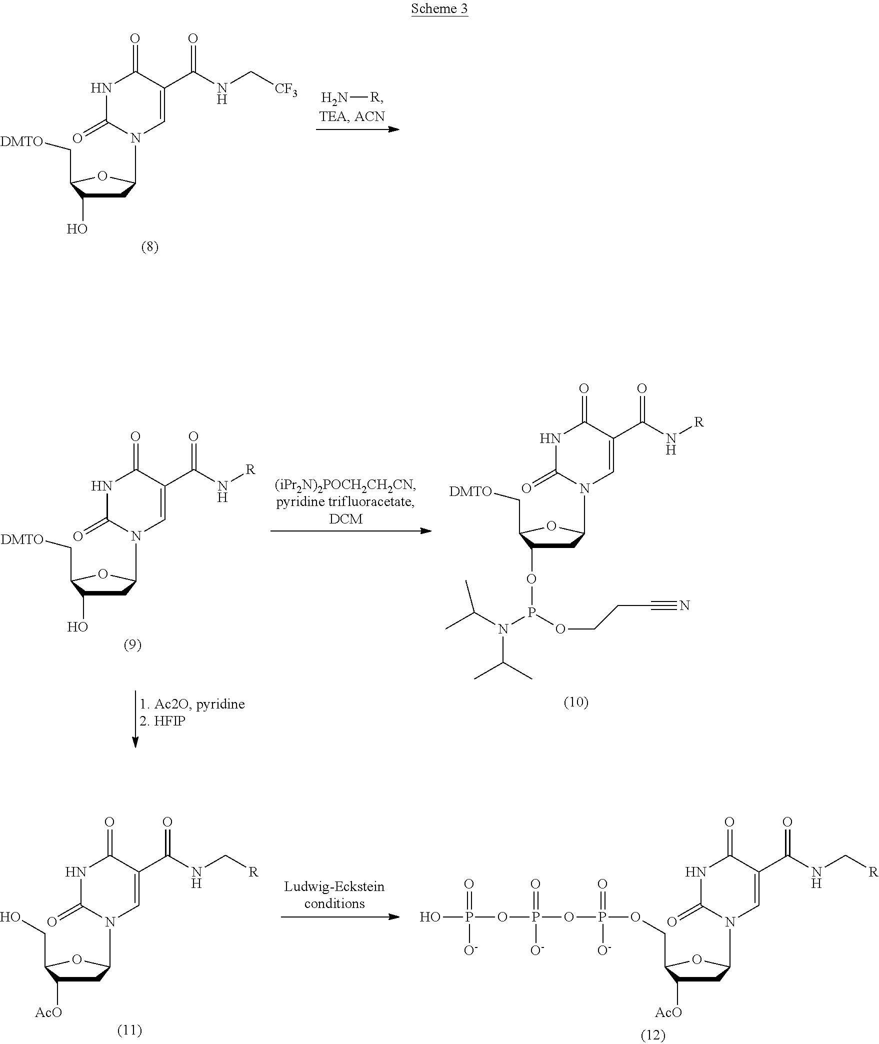

For preparation of 5′-triphosphate reagents (TPP reagent, Scheme 3, product 12), the 5′O-DMT-protected nucleosides (Scheme 3, product 9) were acetylated with acetic anhydride in pyridine, followed by cleavage of the DMT with 1,1,1,3,3,3-hexafluoro-2-propanol (HFIP, Scheme 3, product 11) (see, e.g., Leonard and Neelima, 1995). The resulting crystalline 3′-O-acetylated nucleosides were converted into the crude 5′-O-triphosphates by the Ludwig-Eckstein (Ludwig, J. and Eckstein, F. J. Org. Chem., 1989, 54:631) process (Scheme 3, product 12). These chemically modified nucleotides generally require a two-stage purification process: anion-exchange chromatography (AEX) followed by reversed phase preparative HPLC to obtain the desired product in high purity (>90%).

### Example 2-1: Preparation of 5-[N-(3,3-diphenylpropyl) carboxamide]-2′-deoxyuridine (DPPdU) derivatives (Scheme 3)

Synthesis of 5′-O-(4,4′-Dimethoxytrityl)-5-[N-(3,3-diphenylpropyl) carboxamide]-2′-deoxyuridine (Scheme 3, product 9): The starting material, 5′-O-dimethoxytrityl-5-trifluoroethoxycarbonyl-2′-deoxyuridine (Scheme 3, product 8, 10.55 g, 16.6 mmol)) was charged into a dry, argon-purged round bottomed flask. Dry acetonitrile (22 mL) and 3,3-diphenylpropylamine (4.38 g, 20.7 mmol, 1.25 eq) were added to the flask and the mixture was stirred to dissolve the solids. Triethylamine (4.6 mL, 33.2 mmol, 2 eq) was added to the stirring mixture, which was transferred to a water bath and was heated under an inert atmosphere at 40° C. Reaction progress was monitored by reversed phase HPLC (Waters 2795 HPLC with a 2489 detector and using a Waters Symmetry column, buffer A: 100 mM triethylammonium acetate, buffer B: acetonitrile, gradient: 70% buffer B, isocratic, over 30 minutes). After stirring approximately 6.5 hours, analysis showed the reaction to be complete. The mixture was stirred at room temperature an additional 16 hours, when stirring was discontinued and solvent was evaporated to recover a yellowish foam. The crude mixture was applied to a silica gel flash column equilibrated with 1% triethylamine/79% ethyl acetate/20% hexanes. The product was initially eluted with the same mobile phase, which was modified as the elution progressed to 99% ethyl acetate/1% triethylamine and finally 2% methanol/97% ethyl acetate/1% triethylamine to complete the elution. Product-containing fractions were concentrated to provide a white to off-white foam (11.58 g, 91% yield). ¹H-NMR (300 mHz, CD₃CN): δ=8.65 (t, J=5.9 Hz, 1H), 8.54 (s, 1H), 7.43-7.48 (m, 2H), 7.26-7.383 (m, 14H), 7.16-7.23 (m, 3H), 6.87 (dd, J_{A}=9.0, J_{B}=2.1 Hz, 4H), 6.12 (t, J=6.6 Hz, 1H), 4.29 (td, J_{A}=10.1, J_{B}=3.8 Hz, 1H), 3.98-4.05 (m, 2H), 3.75 (d, J=0.6, 6H), 3.29 (d, J=4.2 Hz, 2H), 3.19-3.27 (m, 2H), 2.19-2.44 (m, 4H). ¹³C-NMR (100 mHz, CD₃CN): δ=163.24 (1C), 7.43-7.48 (m, 2H), 7.26-7.383 (m, 14H), 7.16-7.23 (m, 3H), 6.87 (dd, J_{A}=9.0, J_{B}=2.1 Hz, 4H), 6.12 (t, J=6.6 Hz, 1H), 4.29 (td, J_{A}=10.1, J_{B}=3.8 Hz, 1H), 3.98-4.05 (m, 2H), 3.75 (d, J=0.6, 6H), 3.29 (d, J=4.2 Hz, 2H), 3.19-3.27 (m, 2H), 2.19-2.44 (m, 4H).). MS (m/z) calcd for C₄₆H₄₅N₃O₈, 767.88; found 766.2 [M-H]⁻ (ESI⁻).

Synthesis of 5′-O-(4,4′-Dimethoxytrityl)-4-N-acetyl-5-[N-(3,3-diphenylpropyl) carboxamide]-2′-deoxyuridine-3′-O-(N,N-diisopropyl-O-2-cyanoethylphosphoramidite) (Scheme 3, product 10): In a round-bottomed flask with magnetic stirring, the product of the previous step (Scheme 3, product 9, 10.36 g, 13.5 mmol) was dissolved in anhydrous dichloromethane (34 mL) under argon. To the reaction mixture was added 2-cyanoethyl-N,N,N′,N′-tetraisopropylphosphine (4.5 mL, 14.2 mmol, 1.05 eq) followed by pyridine trifluoroacetate (2.98 g, 14.9 mmol, 1.1 eq). The reaction was stirred for 1.25 hours, then analyzed by thin-layer chromatography (silica gel, eluent: 60% ethyl acetate/40% hexanes), which showed the reaction was complete. The crude mixture was applied to a silica gel flash column equilibrated with 50% ethyl acetate/49% hexanes/1% triethylamine and product elution was achieved using increasing concentrations of ethyl acetate, with the final fractions being eluted using 59% ethyl acetate/40% hexanes/1% triethylamine. All mobile phases were chilled to 0° C. and sparged with argon and product was collected into argon-purged bottles. Product-containing fractions were concentrated to provide a white to off-white foam (8.97 g, 69% yield). ¹H-NMR (300 mHz, DMSO-d₆): δ=12.93 (s, 1H), 8.70 (t, J=5.7 Hz, 1H), 8.47/8.46 (s, 1H), 7.12-7.40 (m, 19H), 6.86 (dd, J_{A}=9.0, J_{B}=2.4 Hz, 4H), 6.04-6.13 (m, 1H), 4.28-4.40 (m, 1H), 4.03-4.12 (m, 1H), 3.92-4.01 (m, 1H), 3.69/3.68 (s, 6H), 3.64-3.76 (m, 1H), 3.40-3.62 (m, 4H), 3.11-3.31 (m, 4H), 2.75 (t, J=5.9 Hz, 1H), 2.64 (td, J_{A}=6.0, J_{B}=0.8 Hz, 1H), 2.36-2.47 (m, 2H), 2.26 (dd, J_{A}=14.4, J_{B}=7.2 Hz 2H), 1.10 (dd, J_{A}=12.3, J_{B}=6.6 Hz 9H), 0.95 (d, J=9.3 Hz, 3H). ³¹P-NMR (300 mHz, DMSO-d₆): δ=147.22/147.58 (s, 1P). MS (m/z) calcd for C₅₅H₆₂N₅O₉P, 968.10; found 966.3 [M-H]⁻ (ESI⁻).

Synthesis of 5-[N-(3,3-diphenylpropyl) carboxamide]-3′-O-acetyl-2′-deoxyuridine nucleoside (Scheme 3, product 11): In a round-bottomed flask with magnetic stirring, the starting material (Scheme 3, product 9, 0.953 g, 1.24 mmol) was dissolved in anhydrous pyridine (10 mL) under argon. Acetic anhydride (1 mL, 10.5 mmol, 8.5 eq) dropwise to the stirring mixture. The reaction was stirred 25 hours at room temperature and reaction completion was verified by thin layer chromatography (TLC, 80% ethyl acetate/20% hexanes).

The crude mixture was evaporated, with a co-evaporation from toluene to recover a pale yellow to tan foam. The residue was dissolved in 1,1,1,3,3,3-hexafluoro-2-propanol (HFIP, 10 mL, 95 mmol)) (Leonard, N. J. Tetrahedron Letters, 1995, 36:7833) and heated at approximately 50° C. for 18 hours. Complete cleavage of the DMT group was confirmed by TLC (5% methanol/dichloromethane). The red solution was quenched by pouring into well-stirred methanol (approximately 50-75 mL). The resulting yellow solution was concentrated in vacuo and the residue was dissolved in hot ethyl acetate (10-20 mL). The product crystallized upon cooling and the resulting slurry was stirred at 0° C., followed by filtration and washing with ethyl acetate. The 3′-O-acetyl-nucleoside (product 11) was isolated as a white solid (0.40 g, 64% yield). ¹H-NMR (400 mHz, DMSO-d₆): δ=11.94 (s, 1H), 8.77 (bt, J=5.8 Hz, 1H), 8.72 (s, 1H), 7.22-7.35 (m, 8H), 7.16 (t, J=6.8 Hz, 2H), 6.14 (t, J=6.8 Hz, 1H), 5.22-5.26 (m, 1H), 5.19 (t, J=4.4 Hz, 1H), 4.63 (d, J=5.6 Hz, 2H), 4.10 (dd, J_{A}=5.2, J_{B}=3.2 Hz, 1H), 3.58-3.67 (m, 2H), 2.31-2.41 (m, 2H), 2.26 (dd, J_{A}=14.0, J_{B}=7.2 Hz, 2H), 2.06 (s, 3H). ¹³C-NMR (100 mHz, DMSO-d₆): δ=170.46 (1C), 163.69 (1C), 161.81 (1C), 150.07 (1C), 146.15 (1C), 144.99 (2C), 128.93 (4C), 128.02 (4C), 126.61 (2C), 106.14 (1C), 85.85 (1C), 85.77 (1C), 75.23 (1C), 61.65 (1C), 48.70 (1C), 38.05 (1C), 37.84 (1C), 34.97 (1C), 21.30 (1C). MS (m/z) calcd for C₂₇H₂₉N₃O₇, 507.54; found 506.1 [M-H]⁻ (ESI⁻).

Synthesis of 5-[N-(3,3-diphenylpropyl)carboxamide]-2′-deoxyuridine-5′-O-triphosphate (tris-triethylammonium salt) (Scheme 3, product 12): The triphosphate (12) was synthesized from the 3′-O-acetyl-nucleoside (11) by the procedure of Ludwig and Eckstein (Ludwig, J. and Eckstein, F. J. Org. Chem. 1989, 54:631) at 500 μmol-scale (5×). The crude triphosphate product, after ammonolysis and evaporation, was purified by anion exchange chromatography and reversed phase chromatography, as described in the General Procedures (above). [εest. 13.700 cm⁻¹ M⁻¹] the isolated purified product was 59.8 μmol (12% yield). ¹H-NMR (300 mHz, D₂O): δ=7.95 (s, 1H), 7.22-7.28 (m, 4H), 7.11-7.19 (m, 4H), 6.96-7.05 (d, 2H), 6.05 (t, J=6.9 Hz, 1H), 4.51 (quintet, J=3.0 Hz, 1H), 4.10-4.18 (m, 3H), 3.92 (t, J=7.5 Hz, 1H), 3.17-3.40 (m, 2H), 2.90 (q, J=7.5 Hz, 19H), 2.25-2.38 (m, 3H), 2.11-2.22 (m, 1H), 1.11 (t, J=7.5 Hz, 29H). ³¹P-NMR (100 mHz, D₂O): δ=−9.93 (d, J=20.3 Hz, 1P), −11.63 (d, J=20.9 Hz, 1P), −11.63 (d, J=20.9 Hz, 1P). MS (m/z) calcd for C₂₅H₂₉N₃O₁₅P₃, 704.43; found 704.1M-H]⁻ (ESI⁻).

### Example 2-1: Preparation of 5-{N-[2-(4-biphenylethyl) carboxamide]}-2′-deoxyuridine (BPEdU) derivatives (Scheme 3)

Synthesis of 5′-O-(4,4′-Dimethoxytrityl)-5-{N-[2-(4-biphenylethyl) carboxamide]}-2′-deoxyuridine (Scheme 3, product 9): The starting material, 5′-O-dimethoxytrityl-5-trifluoroethoxycarbonyl-2′-deoxyuridine (Scheme 3, product 8, 10.45 g, 15.9 mmol)) was charged into a dry, argon-purged round bottomed flask. Dry acetonitrile (20 mL) and 2-(4-biphenyl) ethylamine (3.81 g, 19.1 mmol, 1.2 eq) were added to the flask and the mixture was stirred to dissolve the solids. Triethylamine (4.4 mL, 31.8 mmol, 2 eq) was added to the stirring mixture, which was transferred to a water bath and was heated under an inert atmosphere at 40° C. Reaction progress was monitored by thin-layer chromatography (silica gel, eluent: 8% methanol/dichloromethane) and reversed phase HPLC (Waters 2795 HPLC with a 2489 detector and using a Waters Symmetry column, buffer A: 100 mM triethylammonium acetate, buffer B: acetonitrile, gradient: 70% buffer B, isocratic, over 30 minutes). After stirring approximately 6 hours, analysis showed the reaction still incomplete. The mixture was stirred at room temperature an additional 16 hours, re-analyzed and confirmed complete. Stirring was discontinued and solvent was evaporated to recover a yellowish foam. The crude mixture was applied to a silica gel flash column equilibrated with 1% triethylamine/79% ethyl acetate/20% hexanes. The product was initially eluted with the same mobile phase, which was modified as the elution progressed to 0-2% methanol/1% triethylamine/ethyl acetate to complete the elution. Product-containing fractions were concentrated to provide a white to off-white foam (10.89 g, 91% yield). ¹H-NMR (300 mHz, CD₃CN): δ=8.69 (t, J=5.6 Hz, 1H), 8.57 (s, 1H), 7.57-7.67 (m, 4H), 7.44-7.50 (m, 4H), 7.29-7.40 (m, 8H), 7.20-7.26 (m, 1H), 6.86-6.92 (m, 4H), 6.11 (t, J=6.4 Hz, 1H), 4.28 (dt, J_{A}=7.5, J_{B}=3.9 Hz, 1H), 4.01 (dd, J_{A}=8.3, J_{B}=4.3 Hz, 1H), 3.61 (q, J=6.6 Hz, 2H), 3.30 (d, J=4.2 Hz, 2H), 2.90 (t, J=7.1 Hz, 2H), 2.33-2.43 (m, 1H), 2.19-2.29 (m, 1H). ¹³C-NMR (100 mHz, CD₃CN): δ=163.02 (1C), 161.59 (1C), 158.65 (1C), 149.45 (1C), 145.53 (1C), 145.08 (1C), 140.61 (1C), 138.94 (1C), 138.83 (1C), 135.88 (1C), 135.87 (1C), 130.09 (1C), 130.08 (1C), 129.32 (2C), 128.88 (2C), 128.04 (2C), 127.88 (2C), 127.26 (1C), 126.95 (2C), 126.79 (2C), 126.75 (1C), 113.13 (2C), 113.11 (2C), 105.75 (1C), 86.42 (1C), 86.37 (1C), 86.31 (1C), 71.03 (1C), 63.55 (1C), 54.89 (2C), 40.34 (1C), 40.25 (1C), 34.92 (1C). MS (m/z) calcd for C₄₅H₄₃N₃O₈, 753.85; found 745.2 [M-H]⁻ (ESI⁻).

Synthesis of 5′-O-(4,4′-Dimethoxytrityl)-5-{N-[2-(4-biphenylethyl) carboxamide]}-2′-deoxyuridine-3′-O-(N,N-diisopropyl-O-2-cyanoethylphosphoramidite) (Scheme 3, product 10): In a round-bottomed flask with magnetic stirring, the product of the previous step (Scheme 3, product 9, 18.76 g, 24.9 mmol) was dissolved in anhydrous dichloromethane (62 mL) under argon. To the reaction mixture was added 2-cyanoethyl-N,N,N′,N′-tetraisopropylphosphine (8.3 mL, 26.1 mmol, 1.05 eq) followed by pyridine trifluoroacetate (5.40 g, 14.9 mmol, 1.1 eq). The reaction was stirred for 30 minutes, then analyzed by thin-layer chromatography (silica gel, eluent: 5% methanol/dichloromethane), which showed the reaction was complete. The crude mixture was applied to a silica gel flash column equilibrated with 69% ethyl acetate/30% hexanes/1% triethylamine and product elution was achieved using increasing concentrations of ethyl acetate, with the final fractions being eluted using 79% ethyl acetate/20% hexanes/1% triethylamine. All mobile phases were chilled to 0° C. and sparged with argon and product was collected into argon-purged bottles. Product-containing fractions were concentrated to provide a white to off-white foam (20.97 g, 88% yield). ¹H-NMR (400 mHz, DMSO-d₆): δ=11.93 (s, 1H), 8.78 (t, J=5.7 Hz, 1H), 8.52/8.50 (s, 1H), 7.55-7.68 (m, 4H), 7.11-7.49 (m, 16H), 6.84-6.92 (m, 4H), 6.03-6.13 (m, 1H), 4.29-4.41 (m, 1H), 4.07-4.13 (m, 1H), 3.73 (s, 6H), 3.42-3.70 (m, 6H), 3.18-3.31 (m, 2H), 2.83 (t, J=7.2 Hz, 2H), 2.75 (t, J=5.9 Hz, 2H), 2.64 (td, J A=6.0, J_{B}=0.9 Hz, 1.5H), 2.36-2.46 (m, 2H), 1.10 (dd, J_{A}=12.3, J_{B}=6.6 Hz, 12H), 0.96 (d, J=6.9 Hz, 2H). ³¹P-NMR (400 mHz, DMSO-d₆): δ=147.32/147.66 (s, 1P). MS (m/z) calcd for C₅₄H₆₀N₅O₉P, 954.07; found 952.6 [M-H]⁻ (ESI⁻).

Synthesis of 5′-O-(4,4′-Dimethoxytrityl)-5-{N-[2-(4-biphenyl) ethyl carboxamide]}-3′-O-acetyl-2′-deoxyuridine nucleoside (Scheme 3, product 11): In a round-bottomed flask with magnetic stirring, the starting material (Scheme 3, product 9, 0.98 g, 1.30 mmol) was dissolved in anhydrous pyridine (10 mL) under argon. Acetic anhydride (1 mL, 10.5 mmol, 8.1 eq) dropwise to the stirring mixture. The reaction was stirred 25 hours at room temperature and reaction completion was verified by thin layer chromatography (TLC, 80% ethyl acetate/20% hexanes). The crude mixture was evaporated to recover a pale yellow to tan foam. The residue was dissolved in 1,1,1,3,3,3-hexafluoro-2-propanol (10 mL, 95 mmol)) (Leonard, N. J. Tetrahedron Letters, 1995, 36:7833) and heated at approximately 50° C. for 17 hours. Complete cleavage of the DMT group was confirmed by TLC (5% methanol/dichloromethane). The red solution was quenched by pouring into well-stirred methanol (approximately 25-30 mL). Solids began forming almost immediately; stirred the mixture approximately 18 hours, when product was recovered by filtration using cold isopropyl ether to wash the filter cake. The 3′-O-acetyl-nucleoside (product 11) was isolated as a white solid (0.680 g, ˜100% yield). ¹H-NMR (500 mHz, DMSO-d₆): δ=11.99 (s, 1H), 8.80 (t, J=5.5 Hz, 1H), 8.76 (s, 1H), 7.64 (d, J=7.5 Hz, 2H), 7.60 (d, J=8.0 Hz, 2H), 7.45 (t, J=7.8 Hz, 2H), 7.31-7.37 (m, 4H), 6.14 (t, J=6.8 Hz, 1H), 5.22-5.26 (m, 1H), 5.19 (t, J=4.5 Hz, 1H), 4.09 (d, J=1.5 Hz, 1H), 3.62 (dd, J_{A}=7.5, J_{B}=4.0 Hz, 2H), 3.62 (dd, J_{A}=7.5, J_{B}=4.0 Hz, 2H), 2.84 (t, J=7.0 Hz, 2H), 2.30-2.40 (m, 2H), 2.06 (s, 3H). ¹³C-NMR (100 mHz, CD₃CN): δ=170.50 (1C), 165.59 (1C), 161.85 (1C), 150.03 (1C), 146.30 (1C), 140.42 (1C), 139.05 (1C), 138.54 (1C), 129.72 (2C), 129.37 (1C), 127.70 (1C), 127.14 (2C), 126.96 (2C), 106.03 (1C), 85.87 (1C), 85.80 (1C), 75.25 (1C), 61.65(1C), 40.57 (1C), 38.10 (1C), 35.33 (1C), 21.30 (1C). MS (m/z) calcd for C₂₆H₂₇N₃O₇, 493.52; found 492.1 [M-H]⁻ (ESI⁻).

Synthesis of 5′-O-(4,4′-Dimethoxytrityl)-5-{N-[2-(4-biphenylethyl) carboxamide]}-2′-deoxyuridine-5′-O-triphosphate (tris-triethylammonium salt) (Scheme 3, product 12): The triphosphate (12) was synthesized from the 3′-O-acetyl-nucleoside (11) by the procedure of Ludwig and Eckstein (Ludwig, J. and Eckstein, F. J. Org. Chem. 1989, 54:631) at 500 μmol-scale (5×). The crude triphosphate product, after ammonolysis and evaporation, was purified by anion exchange chromatography and reversed phase chromatography, as described in the General Procedures (above). [εest. 13.700 cm⁻¹ M⁻¹] the isolated purified product was 182 μmol (35.9% yield). ¹H-NMR (300 mHz, D₂O): δ=8.38 (s, 1H), 7.55-7.61 (m, 5H), 7.40-7.46 (m, 2H), 7.32-7.37 (m, 3H), 6.11 (t, J=6.8 Hz, 1H), 4.53 (quintet, J=3.2 Hz, 1H), 4.16-4.21 (m, 1H), 4.09-4.15 (m, 2H), 3.60 (t, J=6.8 Hz, 2H), 3.14 (q, J=7.2 Hz, 24H), 2.89 (t, J=6.8 Hz, 2H), 2.25-2.40 (m, 2H), 2.26 (m, 1H), 1.22 (t, J=7.2 Hz, 36H). ³¹P-NMR (100 mHz, D₂O): δ=−10.91 (d, J=19.8 Hz, 1P), −11.41 (d, J=19.9 Hz, 1P), −23.32 (t, J=19.8 Hz, 1P). MS (m/z) calcd for C₂₄H₂₇N₃O₁₅P₃, 690.41; found 690.1 [M-H]⁻ (ESI⁻).

### Example 2-3. Preparation of 5-[N-(4-phenoxyphenylmethyl) carboxamide]-2′-deoxyuridine (POPdU) derivatives (Scheme 3)

Synthesis of 5′-O-(4,4′-Dimethoxytrityl)-5-[N-(4-phenoxyphenylmethyl) carboxamide]-2′-deoxyuridine (Scheme 3, product 9): The starting material, 5′-O-dimethoxytrityl-5-trifluoroethoxycarbonyl-2′-deoxyuridine (Scheme 3, product 8, 28.47 g, 43.4 mmol)) was charged into a dry, argon-purged round bottomed flask. Dry acetonitrile (54 mL) and 4-phenoxyphenylmethylamine (10.38 g, 52.1 mmol, 1.2 eq) were added to the flask and the mixture was stirred to dissolve the solids. Triethylamine (12 mL, 86.8 mmol, 2 eq) was added to the stirring mixture. The reaction was stirred at room temperature under an inert atmosphere 22 hours. At that time, reaction progress was evaluated by reversed phase HPLC (Waters 2795 HPLC with a 2489 detector and using a Waters Symmetry column, buffer A: 100 mM triethylammonium acetate, buffer B: acetonitrile, gradient: 70% buffer B, isocratic, over 30 minutes) and found to be complete. Stirring was discontinued and solvent was evaporated to recover a yellowish foam. The crude mixture was applied to a silica gel flash column equilibrated with 99% ethyl acetate/1% triethylamine. The product was initially eluted with the same mobile phase, which was modified as the elution progressed to 0-1.5% methanol/1% triethylamine/ethyl acetate to complete the elution. Product-containing fractions were concentrated to provide a white to off-white foam (28.89 g, 88% yield). ¹H-NMR (400 mHz, DMSO-d₆): δ=9.25 (t, J=6.0 Hz, 1H), 8.48 (s, 1H), 7.40-7.22 (m, 15H), 7.15-7.19 (m, 1H), 7.08-7.17 (m, 1H), 6.92-7.00 (m, 6H), 6.84-6.90 (m, 4H), 6.10 (t, J=6.4 Hz, 1H), 4.40-4.52 (m, 2H), 4.11 (dt, J_{A}=10.4, J_{B}=4.0 Hz, 1H), 3.95 (dd, J_{A}=8.6, J_{B}=4.4 Hz, 1H), 3.70 (d, J=1.6, 6H), 3.19 (d, J=4.4 Hz, 2H), 2.25-2.33 (m, 1H), 2.16-2.24 (m, 1H). ¹³C-NMR (100 mHz, DMSO-d₆): δ=163.39 (1C), 161.21 (1C), 158.49 (1C), 158.47 (1C), 157.31 (1C), 155.88 (1C), 150.42 (1C), 145.83 (1C), 145.34 (1C), 135.93(1C), 135.81 (1C), 134.98 (1C), 130.45 (2C), 130.26 (2C), 130.15 (2C), 129.54 (2C), 123.63 (1C), 118.74 (2C), 118.69 (2C), 113.68/113.67 (4C), 105.56 (1C), 86.38 (1C), 86.31 (1C), 86.20 (1C), 70.88 (1C), 64.09 (1C), 55.41/55.40 (2C), 44.85 (1C), 40.56 (1C). MS (m/z) calcd for C₄₄H₄₁N₃O₉, 755.82; found 754.2 [M-H]⁻ (ESI⁻).

Synthesis of 5′-O-(4,4′-Dimethoxytrityl)-5-[N-(4-phenoxyphenylmethyl) carboxamide]-2′-deoxyuridine-3′-O-(N,N-diisopropyl-O-2-cyanoethylphosphoramidite) (Scheme 3, product 10): In a round-bottomed flask with magnetic stirring, the product of the previous step (Scheme 3, product 9, 29.14 g, 38.6 mmol) was dissolved in anhydrous dichloromethane (100 mL) under argon. To the reaction mixture was added 2-cyanoethyl-N,N,N′,N′-tetraisopropylphosphine (12 mL, 40.5 mmol, 1.05 eq) followed by pyridine trifluoroacetate (8.30 g, 43.0 mmol, 1.1 eq). The reaction was stirred for 3 hours, then analyzed by thin-layer chromatography (silica gel, eluent: 55% ethyl acetate/45% hexanes), which showed the reaction was complete. The crude mixture was applied to a silica gel flash column equilibrated with 59% ethyl acetate/40% hexanes/1% triethylamine and product elution was achieved with the same mobile phase which was chilled to 0° C. and sparged with argon and product was collected into argon-purged bottles. Product-containing fractions were concentrated to provide a white to off-white foam (30.25 g, 82% yield). ¹H-NMR (300 mHz, DMSO-d₆): δ=11.98 (s, 1H), 9.09 (t, J=6.6 Hz, 1H), 8.53-8.52 (s, 1H), 7.08-7.40 (m, 14H), 6.91-7.00 (m, 4H), 6.82-6.87 (m, 4H), 6.04-6.13 (m, 1H), 4.29-4.55 (m, 3H), 4.05-4.14 (m, 1H), 3.71 (bs, 6H), 3.46-3.70 (m, 4H), 3.18-3.31 (m, 2H), 2.75 (t, J=6.0, 1H), 2.64 (td, J_{A} 6.0, J_{B}=0.3 Hz, 1H), 2.37-2.48 (t, J=5.9 Hz, 2H), 0.97 (d, J=6.9 Hz, 2H). ³¹P-NMR (300 mHz, DMSO-d₆): δ=147.32/147.65 (s, 1P). MS (m/z) calcd for C₅₃H₅₈N₃O₁₀P, 956.05; found 954.3 [M-H]-(ESI⁻).

Synthesis of 5′-O-(4,4′-Dimethoxytrityl)-5-[N-(4-phenoxyphenylmethyl) carboxamide]-2′-deoxyuridine (Scheme 3, product 11): In a round-bottomed flask with magnetic stirring, the starting material (Scheme 3, product 9, 0.79 g, 1.05 mmol) was dissolved in anhydrous pyridine (10 mL) under argon. Acetic anhydride (1 mL, 10.5 mmol, 10 eq) was added dropwise to the stirring mixture. The reaction was stirred 20 hours at room temperature and reaction completion was verified by thin layer chromatography (TLC, 80% ethyl acetate/20% hexanes). The crude mixture was evaporated with a toluene co-evaporation to recover a pale yellow to tan foam. The residue was dissolved in 1,1,1,3,3,3-hexafluoro-2-propanol (HFIP, 10 mL, 95 mmol)) (Leonard, N. J. Tetrahedron Letters, 1995, 36:7833) and heated at approximately 50° C. for 16 hours. Complete cleavage of the DMT group was confirmed by TLC (5% methanol/dichloromethane). The red solution was quenched by pouring into well-stirred methanol (approximately 20-30 mL). A few solids began forming almost immediately. The volume of the mixture was decreased by approximately 50% by evaporation and the mixture was stirred approximately one hour. By that time, the mixture had become thick with solids and product was recovered by filtration using cold isopropyl ether to wash the filter cake. The 3′-O-acetyl-nucleoside (product 11) was isolated as a white solid (0.36 g, 69% yield). ¹H-NMR (400 mHz, DMSO-d₆): δ=11.94 (s, 1H), 9.12 (t, J=6.0 Hz, 1H), 8.79 (s, 1H), 7.27-7.41 (m, 2H), 7.32 (t, J=7.4 Hz, 2H), 7.12 (t, J=7.4 Hz, 1H), 6.92-7.02 (m, 4H), 6.15 (t, J=7.2 Hz, 1H), 5.24 (bt, J=2.4 Hz, 1H), 5.19 (bt, J=4.4 Hz, 1H), 4.46 (d, J=6.0 Hz, 2H), 4.10 (bdd, J_{A}=5.0, J_{B}=3.4 Hz, 1H), 3.58-3.68 (m, 2H), 2.29-2.42 (m, 2H), 2.06 (s, 3H).). ¹³C-NMR (100 mHz, DMSO-d₆): δ=170.48 (1C), 163.63 (1C), 161.96 (1C), 157.32 (1C), 155.91 (1C), 155.91 (1C), 146.49 (1C), 134.78 (1C), 130.47 (2C), 129.60 (2C), 123.75 (1C), 119.20 (2C), 118.83 (2C), 106.10 (1C), 85.91 (1C), 85.81 (1C), 75.22 (1C), 61.65 (1C), 48.04 (1C), 38.12 (1C), 37.84 (1C), 34.97 (1C), 21.30 (1C). MS (m/z) calcd for C₂₅H₂₅N₃O₈, 495.49; found 494.1 [M-H]⁻ (ESI⁻).

Synthesis of 5′-O-(4,4′-Dimethoxytrityl)-5-{N-[2-(4-biphenylethyl) carboxamide]}-2′-deoxyuridine-5′-O-triphosphate (tris-triethylammonium salt) (Scheme 3, product 12): The triphosphate (12) was synthesized from the 3′-O-acetyl-nucleoside (11) by the procedure of Ludwig and Eckstein (Ludwig, J. and Eckstein, F. J. Org. Chem. 1989, 54:631) at 500 μmol-scale (5×). The crude triphosphate product, after ammonolysis and evaporation, was purified by anion exchange chromatography and reversed phase chromatography, as described in the General Procedures (above). [εest. 13.700 cm⁻¹ M-1] the isolated purified product was 192 μmol (38.2% yield). ¹H-NMR (300 mHz, D₂O): δ=8.38 (s, 1H), 7.14-7.26 (m, 4H), 7.05 (t, J=7.5 Hz, 1H), 6.76-6.82 (m, 4H), 6.09 (t, J=6.6 Hz, 1H), 4.5 (quintet, J=3.3 Hz, 1H), 4.39 (q, J=15.3 Hz, 2H), 4.60 (dd, J_{A}=15.1, J_{B}=3.4 Hz, 1H), 4.05-4.17 (m, 3H), 3.08 (q, J=7.5 Hz, 23H), 2.26-2.37 (m, 2H), 1.16 (t, J=7.5 Hz, 35H). ¹³C-NMR (100 mHz, D₂O): δ=163.49 (1C), 156.60 (1C), 155.82 (1C), 150.38 (1C), 146.45 (1C), 133.41(1C), 130.01 (2C), 129.04 (2C), 123.76 (1C), 118.87 (2C), 118.60 (2C), 105.70 (1C), 101.53 (1C), 86.91 (1C), 85.92/85.81 (1C), 70.86 (1C), 65.49 (1C), 46.59 (8C), 42.28 (1C), 38.74 (1C), 22.74 (1C), 8.19 (8C). MS (m/z) calcd for C₂₅H₂₅N₃O₁₆P₃ 692.38; found 692.0 [M-H]⁻ (ESI⁻).

### Example 3: Selection of Aptamers with 5-Position Modified Pyrimidine Nucleotides

This example provides the representative method for the selection and production of DNA aptamers using biphenyl modified dU libraries in direct comparison to a single phenyl modified nucleotide library.

### Preparation of Candidate Mixture

A candidate mixture of partially randomized ssDNA oligonucleotides was prepared by polymerase extension of a DNA primer annealed to a biotinylated ssDNA template (shown in Table 1 below). The candidate mixture contained a 40-nucleotide randomized cassette containing dATP, dGTP, dCTP and either 5-(N-3-phenylpropylcarboxamide)-2′-deoxyuridine triphosphate (PP-dUTP), 5-[N-(4-phenylbenzyl)carboxamide]-2′-deoxyuridine triphosphate (PBn-dUTP), 5-[N-(4-phenoxybenzyl)carboxamide]-2′-deoxyuridine triphosphate (POP-dUTP), 5-{N-[(1,1′-biphenyl)-4-yl)ethyl]carboxyamide}-2′-deoxyuridine triphosphate (BPE-dUTP) or 5-[N-(3,3-diphenylpropyl)carboxamide]-2′-deoxyuridine triphosphate (DPP-dUTP)).

TABLE 1
Sequences of Template and Primers

| Oligonucleotide | | SEQ ID |
|---|---|---|
| Designation | Sequence (5′→3′) | NO: |
| Template 1 | AB'AB' TTT TTT TTT TGC TCT | 1 |
| | GGT CCC GCT CTT G-(N)₄₀-CAG | |
| | TTT CAG GCA GGC TAG GC | |
| Primer 1 | ATA TAT ATG CCT AGC CTG | 2 |
| | CCT GAA ACT G | |
| Primer 2 | AB'AB' TTT TTT TTT TGC TCT | 3 |
| | GGT CCC GCT CTT G | |

B' = biotin

Nine thousand two hundred microliters of a 50% slurry of Streptavidin Plus UltraLink Resin (PIERCE) was washed three times with once with 40 mL of 16 mM NaCl. Resin was resuspended in a final volume of 8.5 mL 16 mM NaCl. Three hundred fifty two nanomoles of template 1 (SEQ ID NO: 1) possessing two biotin residues (designated as B′ in the sequence) and 40 randomized positions (designated as N₄₀ in the sequence) were added to the washed UltraLink SA beads and rotated at 37° C. for 30 minutes. The beads were then washed three times with 16 mM NaCl. Between each wash, the beads were recovered by centrifugation. The beads, now containing the captured template, were suspended in 1.125 mL of extension reaction buffer containing 18 nmol of primer 1 (SEQ ID NO: 2), 1×SQ20 buffer (120 mM Tris-HCl, pH7.8, 10 mM KCl, 7 mM MgSO₄, 6 mM (NH₄)₂SO₄, 0.001% BSA and 0.1% Triton X-100), 112 units of KOD XL DNA Polymerase (EID MILLIPORE), and 1 mM each of dATP, dGTP, dCTP and one of PP-dUTP, PBn-dUTP, POP-dUTP, BPE-dUTP or DPP-dUTP. The beads were allowed to incubate at 68° C. for 2 hours. The beads were then washed three times with 16 mM NaCl. The aptamer libraries were eluted from the beads with 2 mL of 20 mM NaOH. The eluted libraries were immediately neutralized with 52 μL of 1N HCl and 100 μL HEPES pH 7.5 and 2 μL 10% TWEEN-20. The libraries were concentrated with an AMICON Ultracel YM-3 filter to approximately 0.32 mL-0.52 mL and the concentration of library determine by ultraviolet absorbance spectroscopy.

### Immobilization of Target Protein

The following protein targets used in SELEX were purchased from commercial vendors: Interleukin-33 (IL-33) protein, Novoprotein catalog #C₂₃₃; XIAP protein, R&D Systems catalog #895-XB-050; TNF-alpha protein, Acro Biosystems catalog TNA-5228; KRAS (K-Ras) protein, Sino Biological catalog #12259-H07E. The His-tagged generated target proteins were immobilized on His-tag Dynabeads (Thermo Fisher) paramagnetic beads (MyOne SA, Invitrogen, or hereinafter referred to as His beads) for SELEX (Rounds 1 through 7). Beads (40 mgs) were prepared by washing three times with 20 mL of SB18T0.01 buffer, composed of 40 mM HEPES (4-(2-Hydroxyethyl)piperazine-1-ethanesulfonic acid) buffer adjusted to pH 7.5 with NaOH, 102 mM NaCl, 5 mM KCl, 5 mM MgCl2 and 0.01% TWEEN 20). Finally, the beads were suspended at 2.5 mgs/mL in SB18T0.01 and stored at 4° C. until use.

Aptamer Selection with Slow Off-Rate Enrichment Process

A total of seven rounds of the SELEX process were completed with selection for affinity and slow off-rate. Prior to each round a counter selection was performed to reduce background and to reduce the likelihood of obtaining aptamers with nonspecific binding to protein. Counter selections were performed as follows.

For round 1, 100 μL of the DNA candidate mixture containing approximately 1 nmole of DNA in SB18T0.01 was heated at 95° C. for 5 minutes and then cooled to 70° C. for 5 minutes, then to 48° C. for 5 minutes and then transferred to a 37° C. block for 5 minutes. The sample was then combined with 10 μL of protein competitor mixture (0.1% HSA, 10 μM casein, and 10 μM prothrombin in SB18T0.01), and 0.025 mg (10 μL) of His beads coated with HEXA-His (Anaspec, catalog #24420) and incubated at 37° C. for 10 minutes with mixing. Beads were removed by magnetic separation.

For Rounds 2-7, a 65 μL aliquot of the DNA candidate mixture obtained from the previous round (65% of eDNA obtained from previous round) was mixed with 16 μL of 5×SB18T0.01. The sample was heated to 95° C. for 3 minutes and cooled to 37° C. at a rate of 0.1° C. /second. The sample was then combined with 9 μL of protein competitor mixture (0.1% HSA, 10 μM casein, and 10 μM prothrombin in SB18T0.01), and 0.025 mg (10 μL) His beads and incubated at 37° C. for 10 minutes with mixing. Beads were removed by magnetic separation.

Following the first counter selection the target protein was pre-immobilized on His beads for the Round 1 selection process. To accomplish this, 0.125 mg of protein His beads were mixed with 50 μmoles of target protein and incubated for 30 minutes at 37° C. Unbound target was removed by washing the beads with SB18T0.01. The counter-selected-DNA candidate mixture (100 μL) was added to the beads and incubated at 37° C. for 60 minutes with mixing. No slow off-rate enrichment process was employed in the first round and beads were simply washed 5 times with 100 μL SB18T0.01. Following the washes, the bound aptamer was eluted from the beads by adding 170 μL of 2 mM NaOH, and incubating at 37° C. for 5 minutes with mixing. The aptamer-containing-eluate (170 μL) was transferred to a new tube after magnetic separation of the beads and the solution neutralized by addition of 40 μL of neutralization buffer (500 mM Tris-HCl pH 7.5, 8 mM HCl).

For Rounds 2-7, selections were performed with the DNA candidate mixture and target protein as described below while, in parallel, an identical selection was performed with the DNA candidate mixture, but without the target protein. Comparison of the Ct values obtained from PCR for the sample with target protein (signal S) and sample without target protein (background B) were used as a guide to reduce the target concentration in the next round. If the delta Ct value was greater than 4, but less than 8, the target protein was reduced three-fold in the next round. If the delta Ct value was greater than 8, the target was reduced 10-fold in the next round.

For Round 2, labeled target protein (5 μmoles in 10 μL) was mixed with 40 μL of counter selected DNA candidate mixture and incubated at 37° C. for 15 minutes. A slow off-rate enrichment process was begun by adding 50 μL of 10 mM dextran sulfate followed by the immediate addition of 0.0125 mg of His beads. This was allowed to incubate for 15 minutes at 37° C. with mixing. Beads were then washed 5 times with 100 μL of SB18T0.01. The aptamer strand was eluted from the beads by adding 100 μL of sodium perchlorate, and incubating at 37° C. for 10 minutes with mixing. Beads were removed by magnetic separation and 100 μL of aptamer eluate was transferred to a new tube.

Rounds 3 through 7 were performed as described for Round 2 except the amount of target protein was lowered as needed based on the delta Ct values for each target and library combination. The dextran sulfate was added 15 minutes (rounds 3 and 4), 30 minutes (rounds 5 and 6), 45 minutes (round 7) prior to the addition of His beads.

For rounds 2 through 7, following the perchlorate elution, 100 μL of the aptamer eluate was captured with 0.0625 mg (25 μL) of SA beads pre-bound with primer 2 (SEQ ID NO: 3), herein after referred to as Primer Beads, and incubated at 50° C. for 10 minutes with shaking followed by a 25° C. incubation for 10 minutes with shaking. The beads were washed 2 times with 100 μL SB18T0.01 and 1 time with 16 mM NaCl. Bound aptamer was eluted with 120 μL water and incubated at 75° C. for 2 minutes. Beads were removed by magnetic separation and 120 μL of aptamer eluate was transferred to a new tube. Primer beads were prepared by resuspending 15 mg SA beads (1.5 mL of 10 mg/mL SA beads washed once with 2 mL 20 mM NaOH, twice with 2 mL SB18T0.01) in 0.5 mL 1 M NaCl, 0.01% tween-20 and adding 7 nmoles primer 2 (SEQ ID NO: 3). The mixture was incubated at 37° C. for 1 hour.

Following incubation, the beads were washed 2 times with 1 mL SB18T0.01 and 2 times with 1 mL 16 mM NaCl. Beads were resuspended to 2.5 mg/ml in 5 M NaCl, 0.01% tween-20.

### Aptamer Amplification and Purification

Selected aptamer DNA from each round was amplified and quantified by QPCR. 48 μL DNA was added to 12 μL QPCR Mix (10×KOD DNA Polymerase Buffer; Novagen #71157, diluted to 5×, 25 mM MgCl2, 5 μM forward PCR primer (Primer 1, SEQ ID NO:2), 5 μM biotinylated reverse PCR primer (Primer 2, SEQ ID NO:3), 5×SYBR Green I, 0.075 U/μL KOD XL DNA Polymerase, and 1 mM each dATP, dCTP, dGTP, and dTTP) and thermal cycled in a Bio-Rad MyIQ QPCR instrument with the following protocol: 1 cycle of 96° C. for 15 seconds and 68° C. for 30 minutes; followed by 25 cycles of 96° C. for 15 seconds, 68° C. for 1 minute. Quantification was done with the instrument software and the number of copies of DNA selected, with and without target protein, was compared to determine signal/background ratios.

Following amplification, the PCR product was captured on SA beads via the biotinylated antisense strand. 25 mL SA beads (10 mg/mL) were washed once with 25 mL 20 mM NaOH, twice with 25 mL SB18T0.01, resuspended in 25 mL SB18T0.01, and stored at 4° C. 25 μL SA beads (10 mg/mL in SB18T0.01) were added to 50 μL double-stranded QPCR products and incubated at 25° C. for 5 minutes with mixing. The “sense” strand was eluted from the beads by adding 100 μL 20 mM NaOH, and incubating at 25° C. for 1 minute with mixing. The eluted strand was discarded and the beads were washed 2 times with SB18T0.01 and once with 16 mM NaCl.

Aptamer sense strand containing either PP-dUTP, PBn-dUTP, POP-dUTP, BPE-dUTP or DPP-dUTP was prepared by primer extension from the immobilized antisense strand. The beads were suspended in 40 μL primer extension reaction mixture (1× Primer Extension Buffer (120 mM Tris-HCl pH 7.8, 10 mM KCl, 7 mM MgSO₄, 6 mM (NH₄)₂SO₄, 0.1% TRITON X-100 and 0.001% bovine serum albumin), 4 μM forward primer (Primer 1, SEQ ID NO: 2), 0.5 mM each dATP, dCTP, dGTP, and either PP-dUTP, PBn-dUTP, POP-dUTP, BPE-dUTP or DPP-dUTP, and 0.075 U/μL KOD XL DNA Polymerase) and incubated at 68° C. for 45 minutes with mixing. The beads were washed 2 times with SB18T0.01, 1 time with 16 mM NaCl and the aptamer strand was eluted from the beads by adding 85 μL of 20 mM NaOH, and incubating at 37° C. for 2 minute with mixing. 83 μL aptamer eluate was transferred to a new tube after magnetic separation, neutralized with 20 μL of 80 mM HCl, buffered with 5 μL of 0.1 M HEPES, pH 7.5.

### Selection Stringency and Feedback

The relative target protein concentration of the selection step was lowered each round in response to the QPCR signal (A Ct) following the rule below:

$$If Δ Ct < 4 , [ P ] ( i + 1 ) = [ P ] ( i ) \\ If 4 \leq Δ Ct < 8 , [ P ] ( i + 1 ) = [ P ] ( i ) / 3.2 \\ If Δ Ct \geq 8 , [ P ] ( i + 1 ) = [ P ] ( i ) / 10$$

Where [P]=protein concentration and i=current round number.

After each selection round, the convergence state of the enriched DNA mixture was determined. 10 μL double-stranded QPCR product was diluted to 200 μL with 4 mM MgCl2 containing 1×SYBR Green I. Samples were analyzed for convergence using a C0t analysis which measures the hybridization time for complex mixtures of double stranded oligonucleotides. Samples were thermal cycled with the following protocol: 3 cycles of 98° C. for 1 minute, 85° C. for 1 minute; 2 cycles of 98° C. for 1 minute, then 85° C. for 30 minutes. During the 30 minutes at 85° C., fluorescent images were measured at 5-second intervals. The fluorescence intensity was plotted as a function of the logarithm of time, and an increased rate of hybridization with each SELEX round was observed, indicating sequence convergence.

### Example 4: Equilibrium Binding Constant (Kd) for Enriched SELEX Pool to Protein Target

This example provides the method used herein to measure SELEX pool-protein binding affinities and to determine Kd.

The binding affinities of the enriched Round 7 SELEX pools are shown in Table 2 below, were determined. Briefly, binding constants (Kd values) of enriched SELEX pools were determined by filter binding assay for binding to recombinant XIAP, IL-33, TNF-alpha and K-Ras proteins. Kd values of enriched SELEX pools were measured in SB18T buffer.

Round 7 enriched SELEX pools were 5′ end labeled using T4 polynucleotide kinase (New England Biolabs) and 7-[32P]ATP (Perkin-Elmer). Radiolabeled aptamers (20,000-40,000 CPM, ˜0.03 nM) were mixed with target proteins at concentrations ranging from 10⁻⁷ to 10-12 M and incubated at 37° C. for 40 minutes.

Following incubation, reactions were mixed with an equal volume of 10 mM Dextran Sulfate and 0.014 mg of His-tag Dyna beads (Invitrogen) and incubated with mixing at 37° C. for 5 minutes. Bound complexes were captured on Durapore filter plates (EMD Millipore) and the fraction of bound aptamer was quantified with a phosphorimager (Typhoon FLA 9500, GE) and data were analyzed in ImageQuant (GE).

To determine binding affinity, data were fit using the equation:

$$y = ( max - min ) ( Protein ) / ( {K}_{d} + Protein ) + min .$$

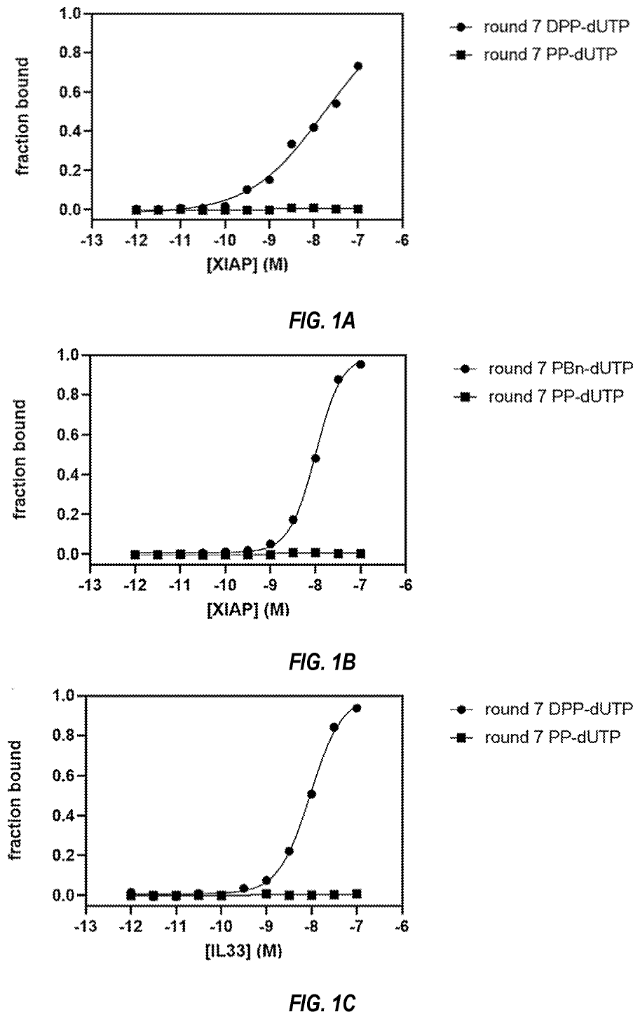

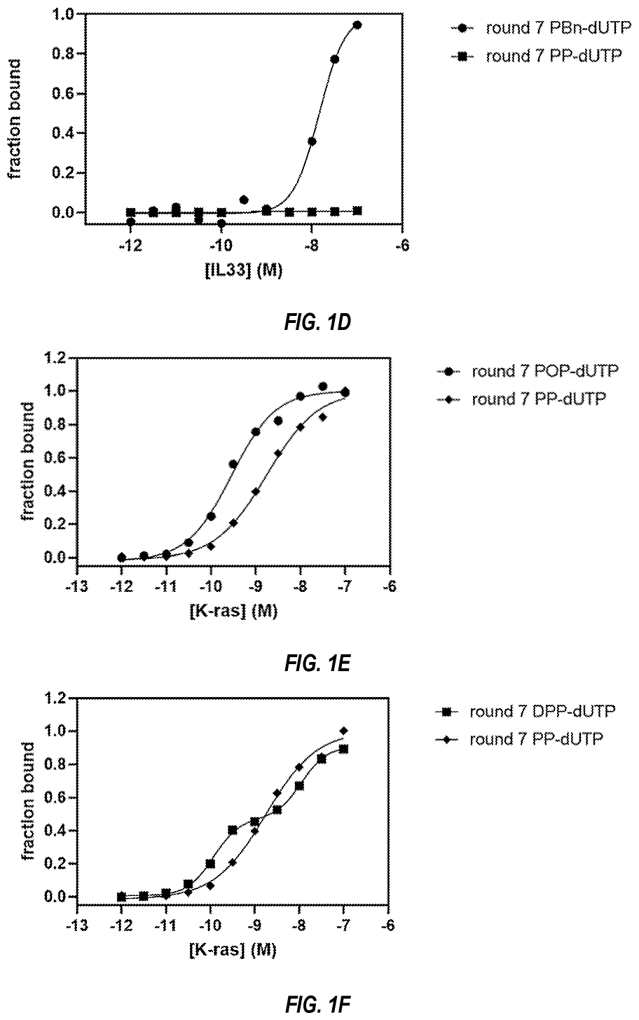

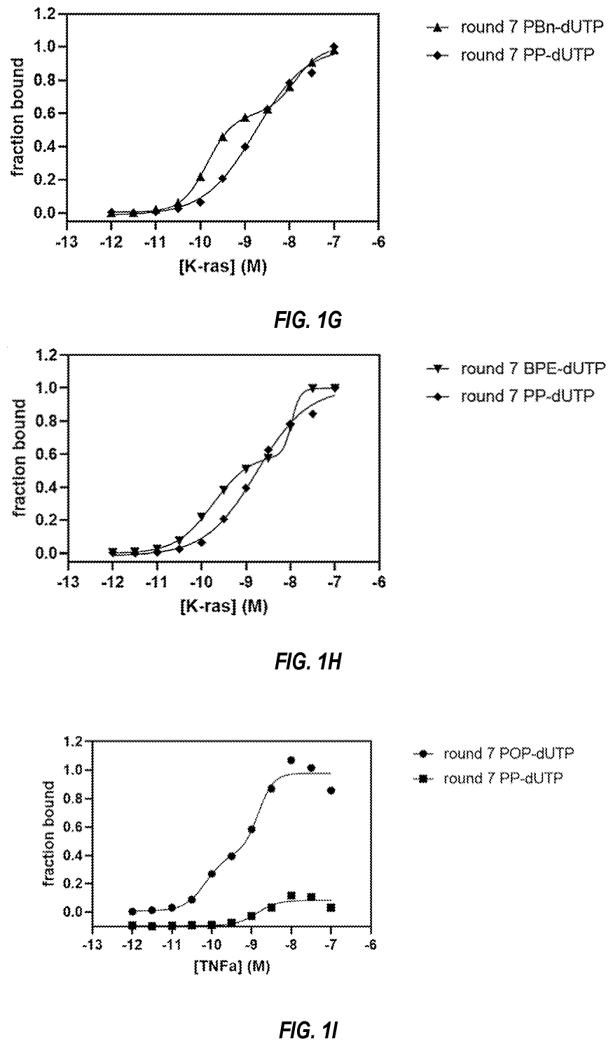

and plotted using GraphPad Prism version 7.00, or data were fit using the four parameter sigmoid dose-response model. All data were plotted using GraphPad Prism version 7.00. Results are shown in FIGS. 1A-1I and Table 2.

TABLE 2

| Target | SELEX round 7 pool | Kd (M) |
|---|---|---|
| XIAP | dATP, dGTP, dCTP, PP-dUTP | >1.0 × 10⁻⁶ |
| XIAP | dATP, dGTP, dCTP, DPP-dUTP | 1.7 × 10⁻⁸ |
| XIAP | dATP, dGTP, dCTP, PBn-dUTP | 1.0 × 10⁻⁸ |
| IL-33 | dATP, dGTP, dCTP, PP-dUTP | >1.0 × 10⁻⁶ |
| IL-33 | dATP, dGTP, dCTP, PBn-dUTP | 9.3 × 10⁻⁹ |
| K-Ras | dATP, dGTP, dCTP, PP-dUTP | 1.7 × 10⁻⁹ |
| K-Ras | dATP, dGTP, dCTP, POP-dUTP | 2.8 × 10⁻¹⁰ |
| K-Ras | dATP, dGTP, dCTP, DPP-dUTP | 1.2 × 10⁻¹⁰ |
| K-Ras | dATP, dGTP, dCTP, PBn-dUTP | 1.4 × 10⁻¹⁰ |
| K-Ras | dATP, dGTP, dCTP, BPE-dUTP | 1.8 × 10⁻¹⁰ |
| TNF-alpha | dATP, dGTP, dCTP, PP-dUTP | 1.4 × 10⁻⁹ |
| TNF-alpha | dATP, dGTP, dCTP, POP-dUTP | 7.2 × 10⁻¹¹ |

### Example 5: Selection of Aptamers with Biphenyl Modified Nucleotides

This example provides the representative method for the selection and production of DNA aptamers using biphenyl modified dC libraries in direct comparison to a single phenyl modified nucleotide library.

### Preparation of Candidate Mixture

A candidate mixture of partially randomized ssDNA oligonucleotides was prepared by polymerase extension of a DNA primer annealed to a biotinylated ssDNA template (shown in Table 3 below). The candidate mixture contained a 40-nucleotide randomized cassette containing dATP, dGTP, dUTP, and one of the following PP-dCTP, Bn-dCTP, DPP-dCTP, PBn-dCTP, or POP-dCTP.

TABLE 3
Sequences of Template and Primers

| Oligonucleotide | | SEQ |
|---|---|---|
| Designation | Sequence (5′→3′) | ID NO: |
| Template 1 | AB'AB' TTTTTTTT CTC CCT | 4 |
| | TCT TCT CTT TCT TCT TC- | |
| | (N)₄₀-GTC ACG GCT CCG | |
| | CGA CAT ATA TAT | |
| Primer 1 | ATA TAT ATG TCG CGG AGC | 5 |
| | CGT GAC | |
| Primer 2 | AB'AB' TTTTTTTT CTC CCT | 6 |
| | TCT TCT CTT TCT TCT TC | |

B' = biotin

Four thousand nine hundred microliters of a 50% slurry of Streptavidin Plus UltraLink Resin (PIERCE) was washed one time with 1×SB 18T buffer and three times with 16 mM NaCl. Resin was equally divided into six tubes and twenty-eight nanomoles of template 1 (SEQ ID NO: 4) possessing two biotin residues (designated as B′ in the sequence) and 40 randomized positions (designated as N₄₀ in the sequence) were added to the washed UltraLink SA beads and rotated at 37° C. for 30 minutes. The beads were then washed three times with 16 mM NaCl. Between each wash, the beads were recovered by centrifugation. The beads, now containing the captured template, were suspended in 1.67 mL of extension reaction buffer [containing 56 nmol of primer 1 (SEQ ID NO: 5), 1×SQ20 buffer (120 mM Tris-HCl, pH7.8, 10 mM KCl, 7 mM MgSO₄, 6 mM (NH₄)₂SO₄, 0.001% BSA and 0.1% Triton X-100), 112 units of KOD XL DNA Polymerase (EMD MILLIPORE), and 1 mM each of dATP, dGTP, dUTP and either Bn-dCTP, PP-dCTP, PBn-dCTP, POP-dCTP, or DPP-dCTP. The beads were allowed to incubate at 71° C. for 2 hours. The beads were then washed three times with 16 mM NaCl. The aptamer libraries were eluted from the beads with 1 mL of 20 mM NaOH. The eluted libraries were immediately neutralized with 15 μL of 1N HCl and 10 μL HEPES pH 7.5 and 1 μL 10% TWEEN-20. The concentration of each library was determine by ultraviolet absorbance spectroscopy.

### Immobilization of Target Protein

The following protein targets used in SELEX were purchased from commercial vendors: TNFα Acro Biosystems catalog TNA-5228; B7-H4,R&D Systems catalog 6576-B7-050; sL-Selectin, R&D Systems catalog 728-LS-100. His-tagged generated target proteins were immobilized on His-tag Dynabeads (Thermo Fisher) paramagnetic beads (MyOne SA, Invitrogen, or hereinafter referred to as His beads) for SELEX (Rounds 1 through 7). Beads (40 mgs) were prepared by washing three times with 20 mL of SB18T0.01. Finally, the beads were suspended at 2.5 mgs/mL in SB18T0.01 and stored at 4° C. until use.

Aptamer Selection with Slow Off-Rate Enrichment Process

A total of eight rounds of the SELEX process were completed with selection for affinity and slow off-rate. Prior to each round a counter selection was performed to reduce background and to reduce the likelihood of obtaining aptamers with nonspecific binding to protein. Counter selections were performed as follows.

For round 1, 100 μL of the DNA candidate mixture containing approximately 1 nmole of DNA in SB18T0.01 was heated at 95° C. for 5 minutes and then cooled to 70° C. for 5 minutes, then to 48° C. for 5 minutes and then transferred to a 37° C. block for 5 minutes. The sample was then combined with 10 μL of protein competitor mixture (0.1% HSA, 10 μM casein, and 10 μM prothrombin in SB18T0.01), and 0.025 mg (10 μL) of His beads coated with HEXA-His (Anaspec, catalog #24420) and incubated at 37° C. for 10 minutes with mixing. Beads were removed by magnetic separation.

For Rounds 2-8, a 65 μL aliquot of the DNA candidate mixture obtained from the previous round (65% of eDNA obtained from previous round) was mixed with 16 μL of 5×SB18T0.01. The sample was heated to 95° C. for 3 minutes and cooled to 37° C. at a rate of 0.1° C. /second. The sample was then combined with 9 μL of protein competitor mixture (0.1% HSA, 10 μM casein, and 10 μM prothrombin in SB18T0.01), and 0.025 mg (10 μL) His beads and incubated at 37° C. for 10 minutes with mixing. Beads were removed by magnetic separation.

Following the first counter selection the target protein was pre-immobilized on His beads for the Round 1 selection process. To accomplish this, 0.125 mg of protein His beads were mixed with 50 μmoles of target protein and incubated for 30 minutes at 37° C. Unbound target was removed by washing the beads with SB18T0.01. The counter-selected-DNA candidate mixture (100 μL) was added to the beads and incubated at 37° C. for 60 minutes with mixing. No slow off-rate enrichment process was employed in the first round and beads were simply washed 5 times with 100 μL SB18T0.01. Following the washes, the bound aptamer was eluted from the beads by adding 170 μL of 2 mM NaOH, and incubating at 37° C. for 5 minutes with mixing. The aptamer-containing-eluate (170 μL) was transferred to a new tube after magnetic separation of the beads and the solution neutralized by addition of 40 μL of neutralization buffer (500 mM Tris-HCl pH 7.5, 8 mM HCl).

For Rounds 2-8, selections were performed with the DNA candidate mixture and target protein as described below while, in parallel, an identical selection was performed with the DNA candidate mixture, but without the target protein. Comparison of the Ct values obtained from PCR for the sample with target protein (signal S) and sample without target protein (background B) were used as a guide to reduce the target concentration in the next round. If the delta Ct value was greater than 4, but less than 8, the target protein was reduced three-fold in the next round. If the delta Ct value was greater than 8, the target was reduced 10-fold in the next round.

For Round 2, labeled target protein (5 μmoles in 10 μL) was mixed with 40 μL of counter selected DNA candidate mixture and incubated at 37° C. for 15 minutes. A slow off-rate enrichment process was begun by adding 50 μL of 10 mM dextran sulfate followed by the immediate addition of 0.0125 mg of His beads. This was allowed to incubate for 15 minutes at 37° C. with mixing. Beads were then washed 5 times with 100 μL of SB18T0.01. The aptamer strand was eluted from the beads by adding 100 μL of sodium perchlorate, and incubating at 37° C. for 10 minutes with mixing. Beads were removed by magnetic separation and 100 μL of aptamer eluate was transferred to a new tube.

Rounds 3 through 8 were performed as described for Round 2 except the amount of target protein was lowered as needed based on the delta Ct values for each target and library combination. The dextran sulfate was added 15 minutes (rounds 3 and 4), 30 minutes (rounds 5 and 6), 45 minutes (rounds 7 and 8) prior to the addition of His beads.

For rounds 2 through 8, following the perchlorate elution, 100 μL of the aptamer eluate was captured with 0.0625 mg (25 μL) of SA beads pre-bound with primer 2 (SEQ ID NO: 6), herein after referred to as Primer Beads, and incubated at 50° C. for 10 minutes with shaking followed by a 25° C. incubation for 10 minutes with shaking. The beads were washed 2 times with 100 μL SB18T0.01 and 1 time with 16 mM NaCl. Bound aptamer was eluted with 120 μL water and incubated at 75° C. for 2 minutes. Beads were removed by magnetic separation and 120 μL of aptamer eluate was transferred to a new tube. Primer beads were prepared by resuspending 120 mg SA beads (1.5 mL of 10 mg/mL SA beads washed once with 2 mL 20 mM NaOH, twice with 2 mL SB18T0.01) in 0.5 mL 1 M NaCl, 0.01% tween-20 and adding 14 nmoles primer 2 (SEQ ID NO: 6). The mixture was incubated at 37° C. for 1 hour. Following incubation, the beads were washed 2 times with 1 mL SB18T0.01 and 2 times with 1 mL 16 mM NaCl. Beads were resuspended to 2.5 mg/ml in 5 M NaCl, 0.01% tween-20.

### Aptamer Amplification and Purification

Selected aptamer DNA from each round was amplified and quantified by QPCR. 48 μL DNA was added to 12 μL QPCR Mix (10×KOD DNA Polymerase Buffer; Novagen #71157, diluted to 5×, 25 mM MgCl2, 5 μM forward PCR primer (Primer 1, SEQ ID NO: 5), 5 μM biotinylated reverse PCR primer (Primer 2, SEQ ID NO: 6), 5×SYBR Green I, 0.075 U/μL KOD XL DNA Polymerase, and 1 mM each dATP, dCTP, dGTP, and dTTP) and thermal cycled in a Bio-Rad MyIQ QPCR instrument with the following protocol: 1 cycle of 96° C. for 15 seconds and 71° C. for 30 minutes; followed by 25 cycles of 96° C. for 15 seconds, 71° C. for 1 minute. Quantification was done with the instrument software and the number of copies of DNA selected, with and without target protein, was compared to determine signal/background ratios.

Following amplification, the PCR product was captured on SA beads via the biotinylated antisense strand. 25 mL SA beads (10 mg/mL) were washed once with 25 mL 20 mM NaOH, twice with 25 mL SB18T0.01, resuspended in 25 mL SB18T0.01, and stored at 4° C. 25 μL SA beads (10 mg/mL in SB18T0.01) were added to 50 μL double-stranded QPCR products and incubated at 25° C. for 5 minutes with mixing. The “sense” strand was eluted from the beads by adding 100 μL 20 mM NaOH, and incubating at 25° C. for 1 minute with mixing.

The eluted strand was discarded and the beads were washed 2 times with SB18T0.01 and once with 16 mM NaCl.

Aptamer sense strand containing either Bn-dCTP, PP-dCTP, PBn-dCTP, POP-dCTP, or DPP-dCTP was prepared by primer extension from the immobilized antisense strand. The beads were suspended in 40 μL primer extension reaction mixture (1× Primer Extension Buffer (120 mM Tris-HCl pH 7.8, 10 mM KCl, 7 mM MgSO₄, 6 mM (NH₄)₂SO₄, 0.1% TRITON X-100 and 0.001% bovine serum albumin), 4 μM forward primer (Primer 1, SEQ ID NO: 5), 0.5 mM each dATP, dUTP, dGTP, and either Bn-dCTP, PP-dCTP, PBn-dCTP, POP-dCTP, or DPP-dCTP, and 0.075 U/μL KOD XL DNA Polymerase) and incubated at 71° C. for 45 minutes with mixing. The beads were washed 2 times with SB18T0.01, 1 time with 16 mM NaCl and the aptamer strand was eluted from the beads by adding 85 μL of 20 mM NaOH, and incubating at 37° C. for 2 minute with mixing. 83 μL aptamer eluate was transferred to a new tube after magnetic separation, neutralized with 20 μL of 80 mM HCl, buffered with 5 μL of 0.1 M HEPES, pH 7.5.

### Selection Stringency and Feedback

The relative target protein concentration of the selection step was lowered each round in response to the QPCR signal (A Ct) following the rule below:

$$If Δ Ct < 4 , [ P ] ( i + 1 ) = [ P ] ( i ) \\ If 4 \leq Δ Ct < 8 , [ P ] ( i + 1 ) = [ P ] ( i ) / 3.2 \\ If Δ Ct \geq 8 , [ P ] ( i + 1 ) = [ P ] ( i ) / 10$$

Where [P]=protein concentration and i=current round number.

After each selection round, the convergence state of the enriched DNA mixture was determined. 10 μL double-stranded QPCR product was diluted to 200 μL with 4 mM MgCl2 containing 1×SYBR Green I. Samples were analyzed for convergence using a C0t analysis which measures the hybridization time for complex mixtures of double stranded oligonucleotides. Samples were thermal cycled with the following protocol: 3 cycles of 98° C. for 1 minute, 85° C. for 1 minute; 2 cycles of 98° C. for 1 minute, then 85° C. for 30 minutes. During the 30 minutes at 85° C., fluorescent images were measured at 5-second intervals. The fluorescence intensity was plotted as a function of the logarithm of time, and an increased rate of hybridization with each SELEX round was observed, indicating sequence convergence.

### Example 6: Equilibrium Binding Constant (Kd) for Enriched SELEX Pool to Protein Target

The following method was used to measure SELEX pool-protein binding affinities and to determine Kd.

The binding affinities of the enriched Round 6 or Round 8 SELEX pools are shown in Table 4 below, were determined. Briefly, binding constants (Kd values) of enriched SELEX pools were determined by filter binding assay for binding to recombinant TNFα and B7-H4 proteins. Kd values of enriched SELEX pools were measured in SB18T buffer. Round 6 or round 8 enriched SELEX pools were 5′ end labeled using T4 polynucleotide kinase (New England Biolabs) and 7-[32P]ATP (Perkin-Elmer). Radiolabeled aptamers (20,000-40,000 CPM, ˜0.03 nM) were mixed with target proteins at concentrations ranging from 10⁻⁷ to 10-12 M and incubated at 37° C. for 40 minutes. Following incubation, reactions were mixed with an equal volume of 10 mM Dextran Sulfate and 0.014 mg of His-tag Dyna beads (Invitrogen) and incubated with mixing at 37° C. for 5 minutes (TNFα and sL-Selectin). For B7-H4 protein, 2.2 mg of Zorbax resin (Agilent Technologies) was added to each reaction. Bound complexes were captured on Durapore filter plates (EMD Millipore) and the fraction of bound aptamer was quantified with a phosphorimager (Typhoon FLA 9500, GE) and data were analyzed in ImageQuant (GE).

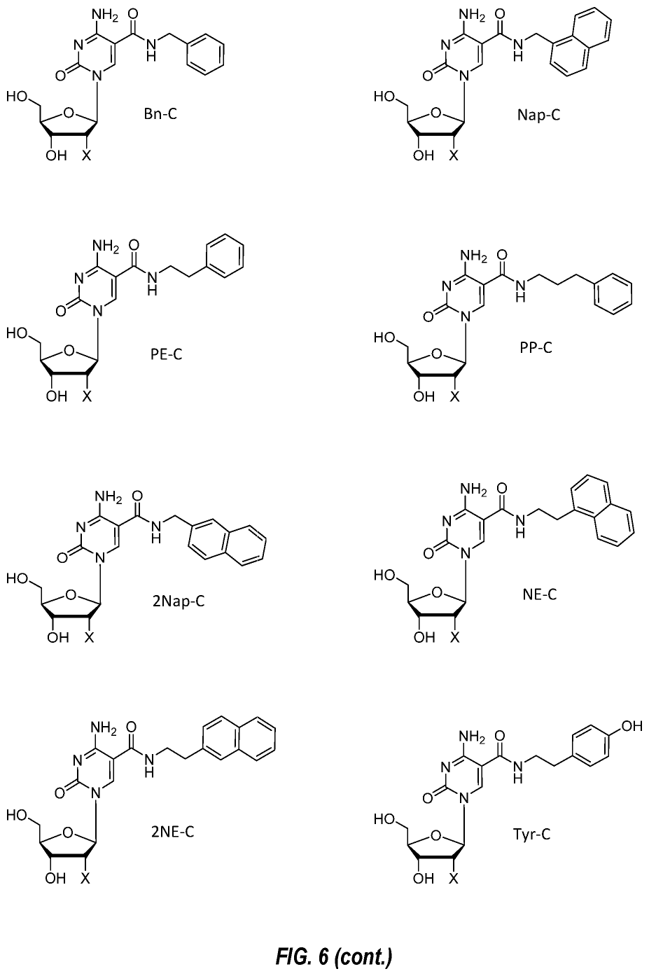

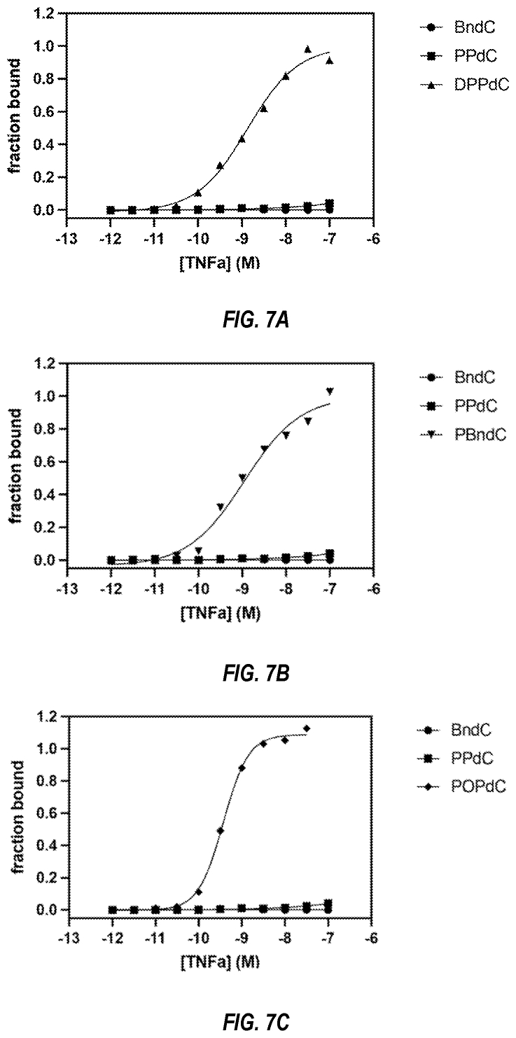

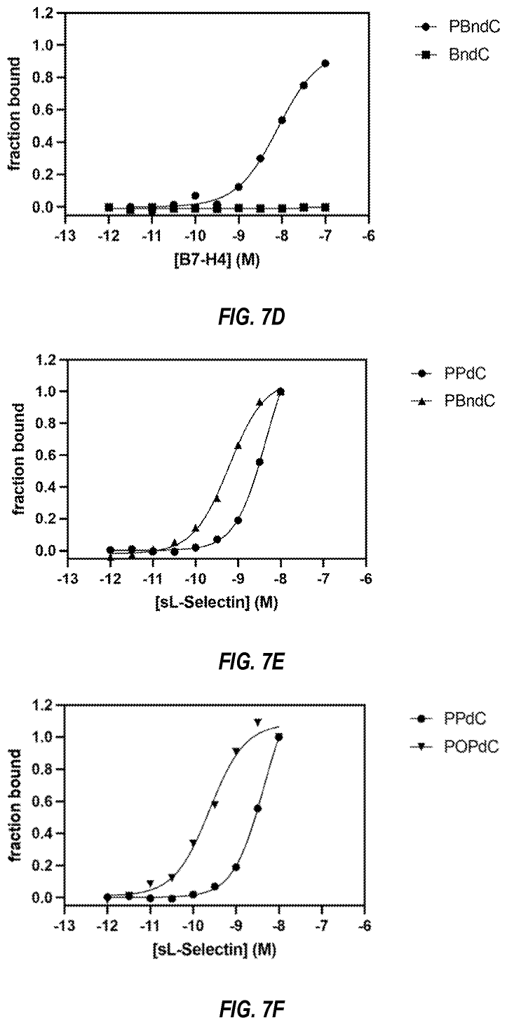

To determine binding affinity, data were fit using the equation: y=(max−min)(Protein)/(K_{d}+Protein)+min. and plotted using GraphPad Prism version 7.00, or data were fit using the four parameter sigmoid dose-response model. All data were plotted using GraphPad Prism version 7.00. Results are shown in FIGS. 7A-7F and Table 4.

TABLE 4

| Target | SELEX round 6 or round 8 pool | Kd (M) |
|---|---|---|
| TNFα | dATP, dGTP, dUTP, PP-dCTP | >1.0 × 10⁻⁶ |
| TNFα | dATP, dGTP, dUTP, Bn-dCTP | >1.0 × 10⁻⁶ |
| TNFα | dATP, dGTP, dUTP, DPP-dCTP | 1.3 × 10⁻⁹ |
| TNFα | dATP, dGTP, dUTP, PBn-dCTP | 1.1 × 10⁻⁹ |
| TNFα | dATP, dGTP, dUTP, POP-dCTP | 3.7 × 10⁻¹⁰ |
| B7-H4 | dATP, dGTP, dUTP, Bn-dCTP | >1.0 × 10⁻⁶ |
| B7-H4 | dATP, dGTP, dUTP, PBn-dCTP | 8.0 × 10⁻⁹ |
| sL-Selectin | dATP, dGTP, dUTP, PP-dCTP | 4.5 × 10⁻⁹ |
| sL-Selectin | dATP, dGTP, dUTP, PBn-dCTP | 6.1 × 10⁻¹⁰ |
| sL-Selectin | dATP, dGTP, dUTP, POP-dCTP | 2.4 × 10⁻¹⁰ |

### Results

Following the completion of round eight of SELEX the affinity enriched pools from rounds six and eight were analyzed in a filter binding assay to identify those pools containing high affinity binding sequences and to assess the value of biphenyl dC modifications (DPP-dC, PBn-dC, POP-dC) compared to single phenyl ring dC modifications (Bn-dC, PP-dC) in SELEX. We found that the biphenyl dC modifications of DPP-dC, PBn-dC and POP-dC showed improved outcomes in SELEX pool affinity binding. The TNFα protein had no measurable binding affinity up to a protein concentration of 1.0×10⁻⁷ M for Bn-dC and PP-dC modified round eight SELEX pools. However, high affinity binding of the DPP-dC, PBn-dC and POP-dC round eight pools (Table 4) were measured, indicating these modified libraries were successful in SELEX. Similarly, the round six pools for B7-H4 protein had no measurable binding affinity for Bn-dC whereas the PBn-dC modified pool had a high affinity of 8.0×10⁻⁹ M. The sL-Selectin protein had modest PP-dC binding affinity for the round 8 pool (4.5×10⁻⁹ M), while the biphenyl libraries of PBn-dC and POP-dC had significantly improved affinities (6.1×10⁻¹⁰ M and 2.4×10⁻¹⁰ M, respectively). These results indicate the biphenyl library pools are enriched for higher affinity sequences than the single phenyl PP-dC pool. In total these results show that biphenyl modified dC libraries result in improved outcomes in SELEX.

1. (canceled)

2. (canceled)

3. (canceled)

4. (canceled)

5. (canceled)

6. (canceled)

7. (canceled)

8. (canceled)

9. A compound comprising the structure of Formula IA or Formula IB: 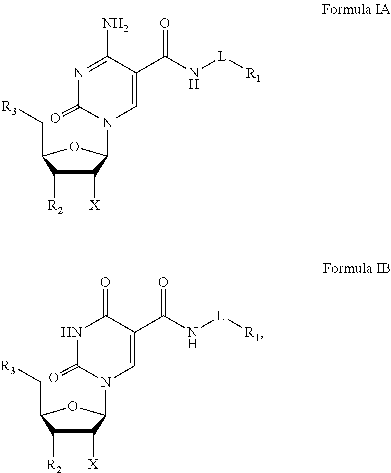
or a salt of either one of these, wherein
each L is independently a —(CH₂)ₙ—, wherein n is 0, 1, 2, 3, 4, 5, 6, 7, 8, 9, or 10;
each R₁ is independently selected from 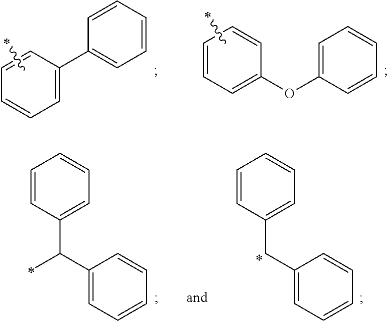
wherein * denotes the point of attachment of the R₁ group to the L group;
each X is independently selected from —H, —OH, —OMe,
—O-allyl, —O-ethyl, —O-propyl, —OCH₂CH₂OCH₃, -fluoro, tert-butyldimethylsilyloxy, —NH₂, and -azido;
each R₂ is independently selected from —OH; -acetyl; —OBz;
—OP(N(CH₂CH₃)₂)(OCH₂CH₂CN), —OP(N(Rₓ)₂)(OCH₂CH₂CN), wherein each Rₓ is independently (C₁₋₆)alkyl; tert-butyldimethylsilyloxy; —O-ss; —OR; —SR; —ZP(Z′)(Z″)—O—R; wherein ss is a solid support, Z, Z′, and Z″ are each independently selected from O and S, and R is an adjacent nucleotide;
each R₃ is independently selected from —OH, —O-trityl, —O-4,4′-dimethoxytrityl, —O-triphosphate, —OR, —SR, —NH₂, —NHR, and —Z—P(Z′)(Z″)O—R, wherein Z, Z′, and Z″ are each independently selected from O and S, and R is an adjacent nucleotide.

10. The compound of claim 9, wherein n is 1, 2, or 3.

11. (canceled)

12. (canceled)

13. The compound of claim 9, wherein each R₁ is independently selected from

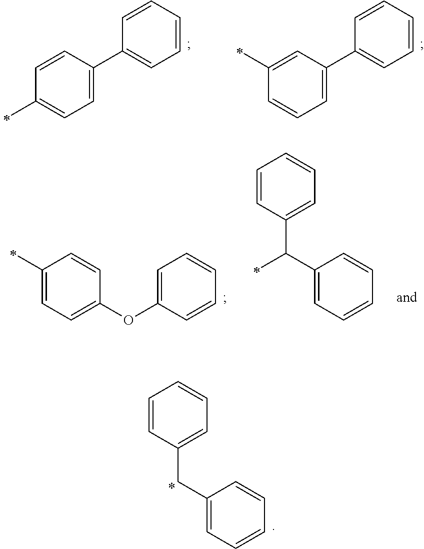

14. (canceled)

15. A compound comprising the structure of Formula IIA or Formula IIB: 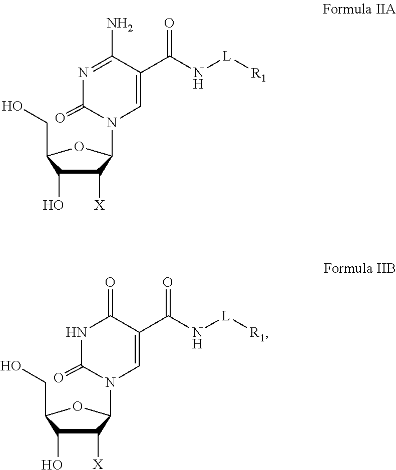
or a salt of either one of these, wherein
each L is independently a —(CH₂)ₙ—, wherein n is 0, 1, 2, 3, 4, 5, 6, 7, 8, 9, or 10;
each R₁ is independently selected from 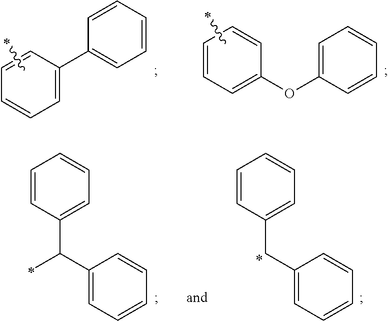
wherein * denotes the point of attachment of the R₁ group to the L group;
each X is independently selected from —H, —OH, —OMe,
—O-allyl, —O-ethyl, —O-propyl, —OCH₂CH₂OCH₃, -fluoro, tert-butyldimethylsilyloxy, —NH₂, and -azido.

16. The compound of claim 15, wherein n is 1, 2, or 3.

17. (canceled)

18. (canceled)

19. The compound of claim 15, wherein each R₁ is independently selected from:

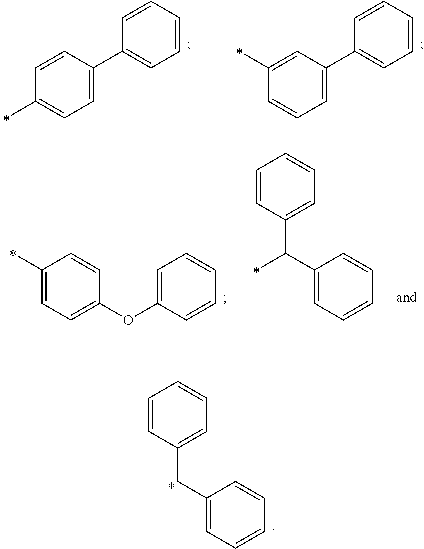

20. A compound comprising the following structure: 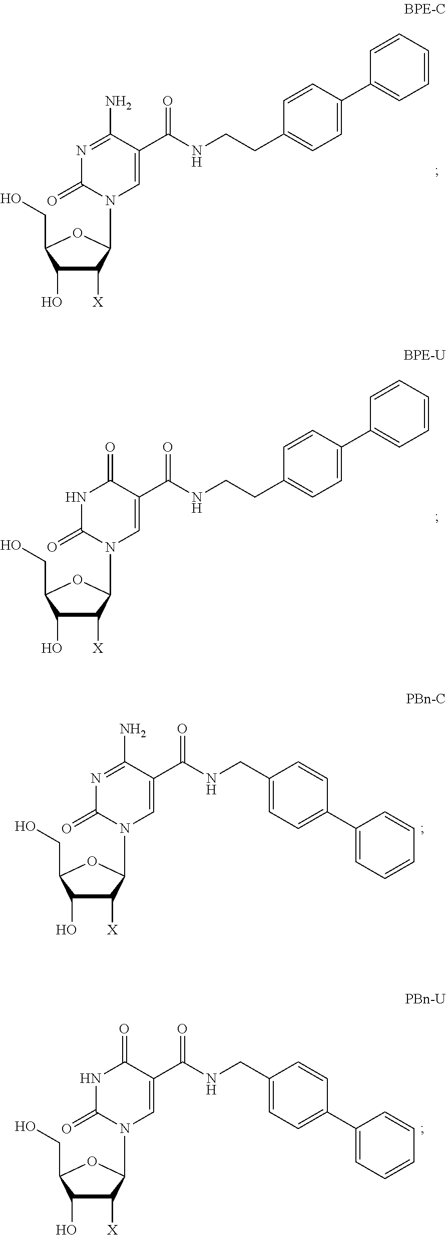 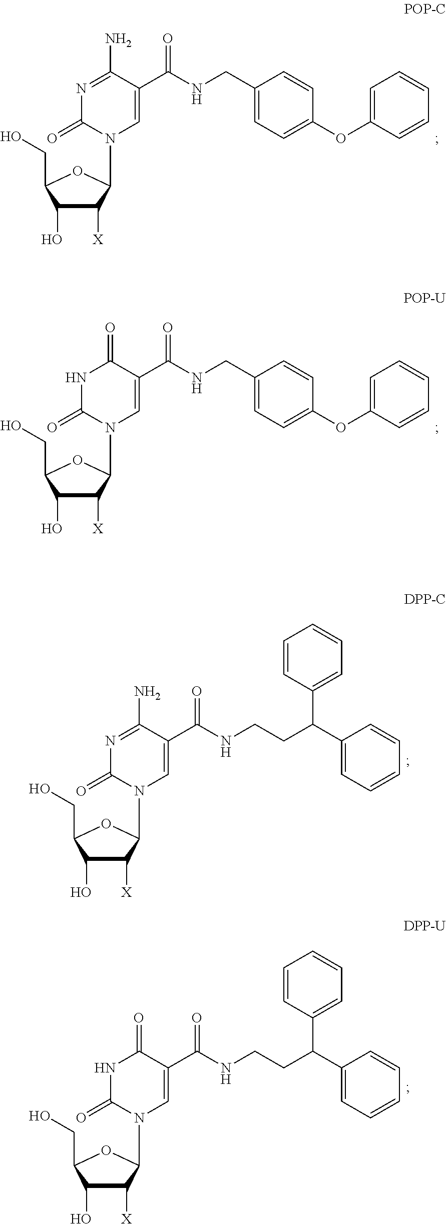 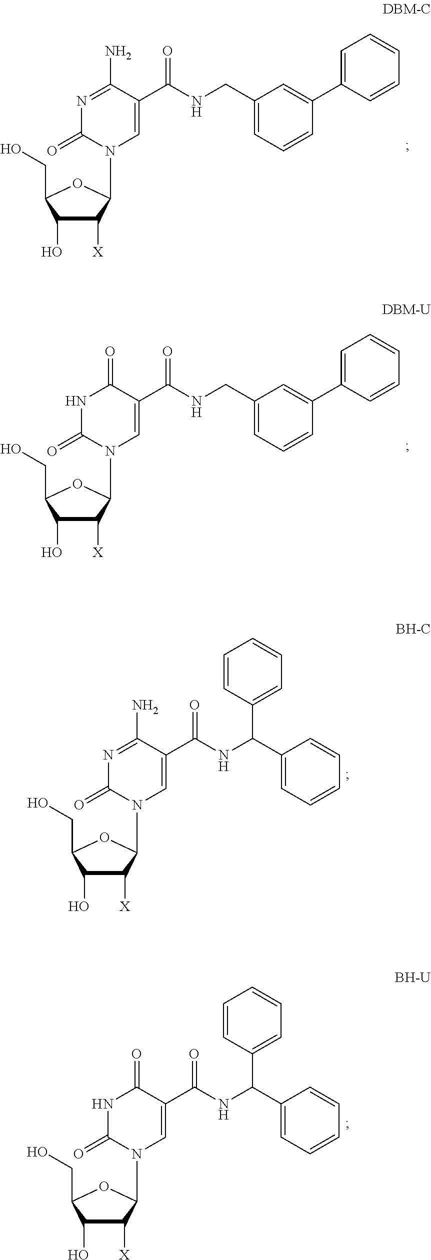
or a salt of any one of these;
wherein each X is independently selected from —H, —OH,
—O-methyl, —O-allyl, —O-ethyl, —O-propyl, —OCH₂CH₂OCH₃, -fluoro, tert-butyldimethylsilyloxy, —NH₂, and -azido.

21. (canceled)

22. (canceled)

23. An oligonucleotide comprising the compound of claim 9.

24. (canceled)

25. (canceled)

26. The oligonucleotide of claim 23, which is an aptamer that binds a target.

27. (canceled)

28. (canceled)

29. (canceled)

30. (canceled)

31. (canceled)

32. (canceled)

33. An aptamer comprising at least one first 5-position modified pyrimidine and at least one second 5-position modified pyrimidine, wherein the first 5-position modified pyrimidine and the second 5-position modified pyrimidine are different 5-position modified pyrimidines, and wherein the at least one first 5-position modified pyrimidine is a compound according to claim 9.

34. (canceled)

35. (canceled)

36. (canceled)

37. A composition comprising a plurality of aptamers of claim 33.

38. (canceled)

39. (canceled)

40. A composition comprising an aptamer and a target, wherein the aptamer and the target are capable of forming a complex, and wherein the aptamer is an aptamer of claim 33.

41. (canceled)

42. The composition of claim 40, wherein the target is selected from a protein, a peptide, a carbohydrate, a small molecule, a cell, and a tissue.

43. (canceled)

44. A pharmaceutical composition comprising at least one aptamer of claim 33, or a pharmaceutically acceptable salt thereof, and a pharmaceutically acceptable carrier.

45. (canceled)

46. A method of treating or preventing a disease or condition in a subject, comprising administering to a subject in need thereof an aptamer of claim 33.

47. (canceled)

48. (canceled)

49. A method comprising:
(a) contacting an aptamer capable of binding to a target molecule with a sample;
(b) incubating the aptamer with the sample to allow an aptamer-target complex to form;
(c) enriching for the aptamer-target complex in the sample; and
(d) detecting for the presence of the aptamer, the aptamer-target complex, or the target molecule, wherein the detection of the aptamer, the aptamer-target complex, or the target molecule indicates that the target molecule is present in the sample, and wherein the lack of detection of the aptamer, the aptamer-target complex, or the target molecule indicates that the target molecule is not present in the sample;
wherein the aptamer comprises an aptamer of claim 33.

50. (canceled)

51. (canceled)

52. (canceled)

53. (canceled)

54. (canceled)

55. (canceled)

56. A method for detecting a target in a sample comprising:
(a) contacting the sample with a first aptamer to form a mixture, wherein the first aptamer is capable of binding to the target to form a first complex;
(b) incubating the mixture under conditions that allow for the first complex to form;
(c) contacting the mixture with a second aptamer, wherein the second aptamer is capable of binding the first complex to form a second complex;
(d) incubating the mixture under conditions that allow for the second complex to form;
(e) detecting for the presence or absence of the first aptamer, the second aptamer, the target, the first complex or the second complex in the mixture, wherein the presence of the first aptamer, the second aptamer, the target, the first complex or the second complex indicates that the target is present in the sample;
wherein the first aptamer comprises a compound of claim 9; and
wherein the second aptamer comprises at least one second 5-position modified pyrimidine;
wherein the first aptamer, the second aptamer and the target are capable of forming a trimer complex.

57. (canceled)

58. (canceled)

59. (canceled)

60. (canceled)

61. A method for identifying one or more aptamers capable of binding to a target molecule comprising:
(a) contacting a library of aptamers with the target molecule to form a mixture, and allowing for the formation of an aptamer-target complex, wherein the aptamer-target complex forms when an aptamer has affinity for the target molecule;
(b) partitioning the aptamer-target complex from the remainder of the mixture (or enriching for the aptamer-target complex);
(c) dissociating the aptamer-target complex; and
(d) identifying the one or more aptamers capable of binding to the target molecule;
wherein the library of aptamers comprises a plurality of polynucleotides, and which is the composition of claim 37.

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. A kit comprising the compound of claim 9, and optionally one or more of (a) a pharmaceutically acceptable carrier, such as a solvent or solution; (b) a pharmaceutically acceptable excipient, such as a stabilizer or buffer; (c) at least one container, vial, or apparatus for holding and/or mixing the kit components; (d) a delivery apparatus: (e) labeling agents useful to detect a target molecule that is bound to an aptamer: (f) a solid support, such as a microarray or bead; and (g) reagents related to quantitation of polymerase chain reaction products, such as intercalating fluorescent dyes or fluorescent DNA probes.

78. (canceled)

79. A compound comprising the structure of Formula III, Formula IV, or Formula V: 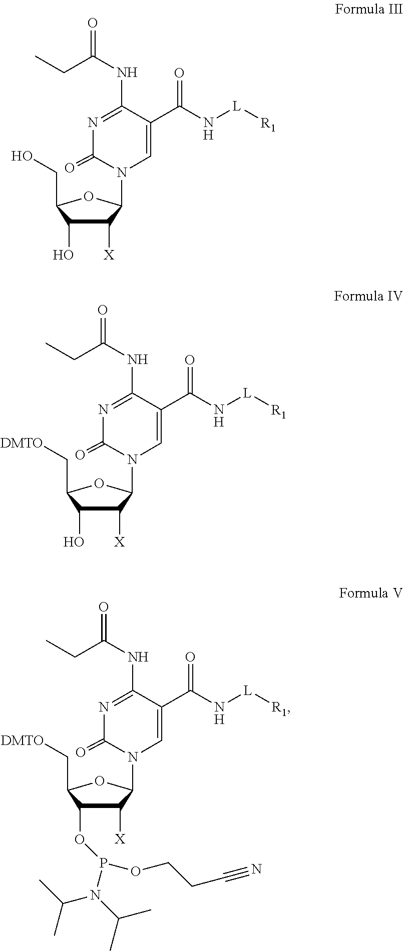
or a salt of any one of these, wherein:
each L is independently a —(CH₂)ₙ—, wherein n is 0, 1, 2, 3, 4, 5, 6, 7, 8, 9, or 10;
each R₁ is independently selected from 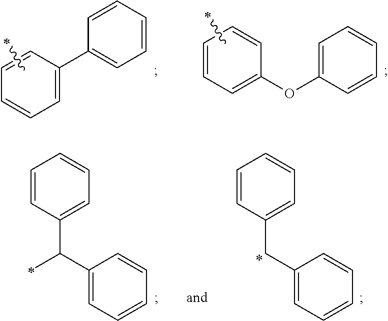
wherein * denotes the point of attachment of the R₁ group to the L group; and
each X is independently selected from —H, —OH, —OMe,
—O-allyl, —O-ethyl, —O-propyl, —OCH₂CH₂OCH₃, -fluoro, tert-butyldimethylsilyloxy, —NH₂, and -azido.

80. (canceled)

81. (canceled)

82. (canceled)

83. (canceled)
